SEQUENCE LISTING (154 sequences)

SEQ ID NO: 1 - DNA, 913 bp, Homo sapiens
acataaatagcaggccaatcccagcccacgcacagacccccaacttgcagctgcccacctcaccctcagctctggcctcttactcaccctctaccacagacatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatagcccagtgagcagcctggagccctggagaccccaccagcctcaccagcgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccaaaaaaaaaaaaaaaaaaaa

SEQ ID NO: 2 - DNA, 684 bp, Homo sapiens
cattcccagcctcacatcactcacaccttgcatttcacccctgcatcccagtcgccctgcagcctcacacagatcctgcacacacccagacagctggcgctcacacattcaccgttggcctgcctctgttcaccctccatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 3 - DNA, 1219 bp, Homo sapiens
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 4 - DNA, 1610 bp, Homo sapiens
agagaacaaaacagaaactcttggaagcaggaaaggtgcatgactcaaagagggaaattcctgtgccataaaaggattgctggtgtataaaatgctctatatatgccaattatcaatttcctttcatgttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 5 - DNA, 1351 bp, Homo sapiens
gtgatggagagcaccagcaaagccttagggcccatccctggcctcctgttacccacagaggggtaggcccttggctctcttccactatgacgtcagcttccattcttcctttcttatagacaattttccatttcaaggaaatcagagcccttaatagttcagtgaggtcactttgctgagcacaatcccatacccttcagcctctgctccacagagcctaagcaaaagatagaaactcacaacttccttgttttgttatctggaaattatcccaggatctggtgcttactcagcatattcaaggaaggtcttacttcattcttccttgattgtgaccatgcccaggctctctgctccctataaaaggcaggcagagccaccgaggagcagagaggttgagaacaacccagaaaccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaagaccaaacggggcaaggaggtctgtgctgaccccaaggagagatgggtcagggattccatgaagcatctggaccaaatatttcaaaatctgaagccatgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggcaaaatgtcattgttctccctcctacctgtctgtagtgttgtggggtcctcccatggatcatcaaggtgaaacactttggtattctttggcaatcagtgctcctgtaagtcaaatgtgtgctttgtactgctgttgttgaaattgatgttactgtatataactatggaattttgaaaaaaaatttcaaaaagaaaaaaatatatataatttaaaactaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 7 - DNA, 2723 bp, Homo sapiens
aaaatgtgttctctaaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataaaaaaaaaaaaaaaaa

SEQ ID NO: 8 - DNA, 760 bp, Homo sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 9 - DNA, 813 bp, Homo sapiens
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 10 - DNA, 798 bp, Homo sapiens
agaaggaggccaggagttgtgagtttccaagccccagctcactctgaccacttctctgcctgcccagcatcatgaagggccttgcagctgccctccttgtcctcgtctgcaccatggccctctgctcctgtgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggctacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatatattggcagtacttattatataaaaggtaaaccagcattctcactgtgaaaaaaaaaaaaaaaaaa

SEQ ID NO: 11 - DNA, 1319 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 12 - DNA, 848 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 13 - DNA, 615 bp, Homo sapiens
gctcagagagaagtgactttgagctcacagtgtcaccgcctgctgatgggagagctgaattcaaaaccagggtgtctccctgagcagagggacctgcacacagagactccctcctgggctcctggcaccatggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttgaagcctcctcaccccagactcctgactgtctcccgggactacctgggacctccaccgttggtgttcaccgcccccaccctgagcgcctgggtccaggggaggccttccagggacgaagaagagccacagtgagggagatcccatccccttgtctgaactggagccatgggcacaaagggcccagattaaagtctttatcctc

SEQ ID NO: 14 - DNA, 894 bp, Homo sapiens
cagtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtgagggaatatgagtgcgtggtgcgagtgcgtgaatattgggggcccggacgcccaggaccccatggcagtgggaaaaatgtaggagactgtttggaaattgattttgaacctgatgaaaataaagaatggaaagcttcagtgctgccgataaa

SEQ ID NO: 15 - DNA, 20 bp, artificial
  Synthetic
gctgaaggtgtgggatgtga

SEQ ID NO: 16 - DNA, 23 bp, artificial
  synthetic
gagtcatcttctccatatccgga

SEQ ID NO: 17 - DNA, 20 bp, artificial
  synthetic
accatggctgctcaaaacct

SEQ ID NO: 18 - DNA, 24 bp, artificial
  Synthetic
gccgtaaaagaggaaacacatcag

SEQ ID NO: 19 - DNA, 20 bp, artificial
  synthetic
acacctctgtctgccagcaa

SEQ ID NO: 20 - DNA, 20 bp, artificial
  synthetic
ggcacctgctttcaagacca

SEQ ID NO: 21 - DNA, 24 bp, artificial
  synthetic
gattgattactttggagttgctgc

SEQ ID NO: 22 - DNA, 23 bp, artificial
  synthetic
catgatgtgaaaaaattcctccc

SEQ ID NO: 23 - DNA, 21 bp, artificial
  synthetic
ggaaggagaaaggctggtcaa

SEQ ID NO: 24 - DNA, 19 bp, artificial
  synthetic
ccaaatcctggagccatcc

SEQ ID NO: 25 - DNA, 21 bp, artificial
  synthetic
gcctgaactgacagaggagca

SEQ ID NO: 26 - DNA, 21 bp, artificial
  synthetic
tttaaacgcccaaagcatcac

SEQ ID NO: 27 - DNA, 21 bp, artificial
  synthetic
tttaaacgcccaaagcatcac

SEQ ID NO: 28 - DNA, 20 bp, artificial
  synthetic
acagttctgcgacaaggcct

SEQ ID NO: 29 - DNA, 19 bp, artificial
  synthetic
ggcctaaacaaggctggca

SEQ ID NO: 30 - DNA, 20 bp, artificial
  synthetic
catcggtcatcttcacgtgg

SEQ ID NO: 31 - DNA, 20 bp, artificial
  synthetic
gcccacactcttcctggaag

SEQ ID NO: 32 - DNA, 22 bp, artificial
  synthetic
cattccagacctcacaccattg

SEQ ID NO: 33 - DNA, 21 bp, artificial
  synthetic
gcagatgttctgaagggatgg

SEQ ID NO: 34 - DNA, 21 bp, artificial
  synthetic
tcacccgcacttcatgttctt

SEQ ID NO: 35 - DNA, 19 bp, artificial
  synthetic
agggagctgggcgagtatg

SEQ ID NO: 36 - DNA, 21 bp, artificial
  synthetic
tgtgtggcggtttttctcagt

SEQ ID NO: 37 - DNA, 20 bp, artificial
  synthetic
agccactggtgaccagaacc

SEQ ID NO: 38 - DNA, 21 bp, artificial
  synthetic
aggaggtggtctctggagctc

SEQ ID NO: 39 - DNA, 20 bp, artificial
  synthetic
cccaggctcttcactgctct

SEQ ID NO: 40 - DNA, 21 bp, artificial
  synthetic
ggttcgagtgatgcatttgga

SEQ ID NO: 41 - DNA, 18 bp, artificial
  synthetic
tttctgcaggaaagcgcg

SEQ ID NO: 42 - DNA, 22 bp, artificial
  synthetic
ctgccaaagccaattgtaacag

SEQ ID NO: 43 - DNA, 22 bp, artificial
  synthetic
atgggagatgggtaaatgacca

SEQ ID NO: 44 - DNA, 23 bp, artificial
  synthetic
ttaaagaagacttgccagggaaa

SEQ ID NO: 45 - DNA, 23 bp, artificial
  synthetic
tgtttgtcttcaccagccttctt

SEQ ID NO: 46 - DNA, 24 bp, artificial
  synthetic
cggaaacacagtgtaaaattctgc

SEQ ID NO: 47 - DNA, 24 bp, artificial
  synthetic
ttaaaaaacctggatcggaaccaa

SEQ ID NO: 48 - DNA, 23 bp, artificial
  synthetic
gcattagcttcagatttacgggt

SEQ ID NO: 49 - DNA, 21 bp, artificial
  synthetic
gctgctttcagcatccaagtg

SEQ ID NO: 50 - DNA, 19 bp, artificial
  synthetic
ccagggacaccgactactg

SEQ ID NO: 51 - DNA, 20 bp, artificial
  Synthetic
aagtgctgccgtcattttct

SEQ ID NO: 52 - DNA, 20 bp, artificial
  Synthetic
cctatggccctcattctcac

SEQ ID NO: 53 - DNA, 23 bp, artificial
  Synthetic
tgcgttttggagctagcggacca

SEQ ID NO: 54 - DNA, 24 bp, artificial
  Synthetic
cgaggaccatacagcacgtgccag

SEQ ID NO: 55 - DNA, 20 bp, artificial
  Synthetic
tccactccctcagaagcact

SEQ ID NO: 56 - DNA, 20 bp, artificial
  Synthetic
agagaagccatgtcggagaa

SEQ ID NO: 57 - DNA, 20 bp, artificial
  Synthetic
tacaccagatccaggggttc

SEQ ID NO: 58 - DNA, 20 bp, artificial
  Synthetic
actcatccaagcgcctatga

SEQ ID NO: 59 - DNA, 19 bp, artificial
  Synthetic
atgatgactctgagcctcc

SEQ ID NO: 60 - DNA, 20 bp, artificial
  Synthetic
gagccctttcctttctttcc

SEQ ID NO: 61 - DNA, 25 bp, artificial
  Synthetic
catcttctcaaaattcgagtgacaa

SEQ ID NO: 62 - DNA, 23 bp, artificial
  Synthetic
tgggagtagacaaggtacaaccc

SEQ ID NO: 63 - DNA, 20 bp, artificial
  Synthetic
taccagctcccaaaatcctg

SEQ ID NO: 64 - DNA, 20 bp, artificial
  Synthetic
cggaatcgtccctttttgta

SEQ ID NO: 65 - DNA, 20 bp, artificial
  Synthetic
tcaaagcccagcacaatgtc

SEQ ID NO: 66 - DNA, 25 bp, artificial
  Synthetic
ttatcgcatagaaaaccagacttgc

SEQ ID NO: 67 - DNA, 28 bp, artificial
  Synthetic
gcagtgtctcagttgcaagacatgtcgg

SEQ ID NO: 68 - DNA, 28 bp, artificial
  Synthetic
cgttggaactggttctccttacagccac

SEQ ID NO: 69 - DNA, 22 bp, artificial
  Synthetic
actgttgcctctcgtacataca

SEQ ID NO: 70 - DNA, 21 bp, artificial
  Synthetic
acccacaatagctctggaagg

SEQ ID NO: 71 - DNA, 23 bp, artificial
  Synthetic
taccatgaggtcacttcagatgc

SEQ ID NO: 72 - DNA, 20 bp, artificial
  Synthetic
gcactctcggcctacattgg

SEQ ID NO: 73 - DNA, 22 bp, artificial
  Synthetic
tgtaatttacccgagtaacggc

SEQ ID NO: 74 - DNA, 23 bp, artificial
  Synthetic
cacctttgtcgtttatgagcctt

SEQ ID NO: 75 - DNA, 20 bp, artificial
  Synthetic
tcgtgccaaatggttacaaa

SEQ ID NO: 76 - DNA, 20 bp, artificial
  Synthetic
acaaggatgtgggttgggta

SEQ ID NO: 77 - DNA, 19 bp, artificial
  Synthetic
gcctcagattatctgccat

SEQ ID NO: 78 - DNA, 21 bp, artificial
  Synthetic
agacacagggctccttctggt

SEQ ID NO: 79 - DNA, 24 bp, artificial
  Synthetic
tcaagtggcatagatgtggaagaa

SEQ ID NO: 80 - DNA, 21 bp, artificial
  Synthetic
tggctctgcaggattttcatg

SEQ ID NO: 81 - DNA, 20 bp, artificial
  Synthetic
atatgggtttcatgggcttg

SEQ ID NO: 82 - DNA, 20 bp, artificial
  Synthetic
gaggctgtaagccaccttga

SEQ ID NO: 83 - DNA, 19 bp, artificial
  Synthetic
tttgcctacctctccctcg

SEQ ID NO: 84 - DNA, 21 bp, artificial
  Synthetic
cgactgcaagattggagcact

SEQ ID NO: 85 - DNA, 19 bp, artificial
  Synthetic
cgggcagatcactacaccc

SEQ ID NO: 86 - DNA, 22 bp, artificial
  Synthetic
tgttactggaacaaagacagcc

SEQ ID NO: 87 - DNA, 21 bp, artificial
  Synthetic
ccttgctgaactgtgaggaga

SEQ ID NO: 88 - DNA, 20 bp, artificial
  Synthetic
cttcgcttacaacgtgtgct

SEQ ID NO: 89 - DNA, 16 bp, artificial
  Synthetic
tccgcgtgcctggaaa

SEQ ID NO: 90 - DNA, 21 bp, artificial
  Synthetic
aagctccgaaataggacctgg

SEQ ID NO: 91 - DNA, 17 bp, artificial
  Synthetic
agttgccttcggactga

SEQ ID NO: 92 - DNA, 20 bp, artificial
  Synthetic
cagaattgccattgcacaac

SEQ ID NO: 93 - DNA, 20 bp, artificial
  Synthetic
gcagaaggaactgcaacaca

SEQ ID NO: 94 - DNA, 20 bp, artificial
  Synthetic
gatggtgaggtgtgcaaatg

SEQ ID NO: 95 - DNA, 19 bp, artificial
  Synthetic
tgccaggaggatgtcacct

SEQ ID NO: 96 - DNA, 20 bp, artificial
  Synthetic
ggcgggtctggtttgatgat

SEQ ID NO: 97 - DNA, 25 bp, artificial
  Synthetic
caaccaacaagtgatattctccatg

SEQ ID NO: 98 - DNA, 21 bp, artificial
  Synthetic
gatccacactctccagctgca

SEQ ID NO: 99 - DNA, 22 bp, artificial
  Synthetic
ctggtgtcgtgaagtcaggaag

SEQ ID NO: 100 - DNA, 22 bp, artificial
  Synthetic
cctcaggaccaggtcagtatcc

SEQ ID NO: 101 - DNA, 19 bp, artificial
  Synthetic
cttctgggcctgctgttca

SEQ ID NO: 102 - DNA, 21 bp, artificial
  Synthetic
ccagcctactcattgggatca

SEQ ID NO: 103 - DNA, 20 bp, artificial
  Synthetic
gcggccaaactgatgaatgt

SEQ ID NO: 104 - DNA, 21 bp, artificial
  Synthetic
cttcttcgtctatggccagga

SEQ ID NO: 105 - DNA, 19 bp, artificial
  Synthetic
cacccgctcgcttctctgt

SEQ ID NO: 106 - DNA, 22 bp, artificial
  Synthetic
gcaacaccttcaagctctggat

SEQ ID NO: 107 - DNA, 20 bp, artificial
  Synthetic
ctcacggaggacggagactc

SEQ ID NO: 108 - DNA, 20 bp, artificial
  Synthetic
ctcttcgtggatgattgcca

SEQ ID NO: 109 - DNA, 20 bp, artificial
  Synthetic
cagcgaggcttcagatttcg

SEQ ID NO: 110 - DNA, 19 bp, artificial
  Synthetic
cacctggcaaacctccatg

SEQ ID NO: 111 - DNA, 24 bp, artificial
  Synthetic
caacagcctgaacatggtgcaact

SEQ ID NO: 112 - DNA, 24 bp, artificial
  Synthetic
attgtgacagttcttgaggccgct

SEQ ID NO: 113 - DNA, 21 bp, artificial
  Synthetic
gttaagcagtacagccccaaa

SEQ ID NO: 114 - DNA, 23 bp, artificial
  Synthetic
agggcatatccaacaacaaactt

SEQ ID NO: 115 - DNA, 22 bp, artificial
  Synthetic
cgcgtctccttcgagctgtttg

SEQ ID NO: 116 - DNA, 24 bp, artificial
  Synthetic
tgtaaagtcaccaccctggcacat

SEQ ID NO: 117 - DNA, 18 bp, artificial
  Synthetic
ccaccccagcaaggagac

SEQ ID NO: 118 - DNA, 20 bp, artificial
  Synthetic
gaaattgtgagggagatgct

SEQ ID NO: 119 - DNA, 20 bp, artificial
  Synthetic
accgttggtgttcaccgccc

SEQ ID NO: 120 - DNA, 20 bp, artificial
  Synthetic
ggccctttgtgcccatggct

SEQ ID NO: 121 - DNA, 20 bp, artificial
  Synthetic
gctactccacctctgcggcg

SEQ ID NO: 122 - DNA, 20 bp, artificial
  Synthetic
cagctgccgtgtgaagccca

SEQ ID NO: 123 - DNA, 20 bp, artificial
  Synthetic
gaggactggtaacggagacg

SEQ ID NO: 124 - DNA, 20 bp, artificial
  Synthetic
gggctgagatctgtttctgg

SEQ ID NO: 125 - DNA, 20 bp, artificial
  Synthetic
ccaccctacacctcctcctt

SEQ ID NO: 126 - DNA, 19 bp, artificial
  Synthetic
agtctgggcagctgaaggt

SEQ ID NO: 127 - DNA, 23 bp, artificial
  Synthetic
tattcctgcaagccaattttgtc

SEQ ID NO: 128 - DNA, 21 bp, artificial
  Synthetic
tcttgatggccttcgattctg

SEQ ID NO: 129 - DNA, 20 bp, artificial
  Synthetic
ctggcgtctaggagagatgg

SEQ ID NO: 130 - DNA, 20 bp, artificial
  Synthetic
ctgggttgacctcgtgagac

SEQ ID NO: 131 - DNA, 20 bp, artificial
  Synthetic
gagaaccaaggtctggtgga

SEQ ID NO: 132 - DNA, 19 bp, artificial
  Synthetic
gagcagaagaaggccagtg

SEQ ID NO: 133 - DNA, 21 bp, artificial
  Synthetic
tgcgtgctgatcgtgatcttc

SEQ ID NO: 134 - DNA, 23 bp, artificial
  Synthetic
ggggtcccaataactgtcatctt

SEQ ID NO: 135 - DNA, 23 bp, artificial
  Synthetic
caacatatcgttggatcacagca

SEQ ID NO: 136 - DNA, 22 bp, artificial
  Synthetic
acagactcactttatgggaacc

SEQ ID NO: 137 - DNA, 19 bp, artificial
  Synthetic
ctgcctcagctgctccaaa

SEQ ID NO: 138 - DNA, 21 bp, artificial
  Synthetic
cggtccactgtgcaagaagag

SEQ ID NO: 139 - DNA, 21 bp, artificial
  Synthetic
ggcgctccccaagaagacagg

SEQ ID NO: 140 - DNA, 21 bp, artificial
  Synthetic
ccaggcactcacctcttccct

SEQ ID NO: 141 - DNA, 21 bp, artificial
  Synthetic
cttcggagtttgggtttgctt

SEQ ID NO: 142 - DNA, 21 bp, artificial
  Synthetic
cattgtggccaaggagatctg

SEQ ID NO: 143 - DNA, 20 bp, artificial
  Synthetic
atgcccagacatctgtgtcc

SEQ ID NO: 144 - DNA, 20 bp, artificial
  Synthetic
ggggtctctatgcccaacaa

SEQ ID NO: 145 - DNA, 21 bp, artificial
  Synthetic
gctgctcagattggagactca

SEQ ID NO: 146 - DNA, 20 bp, artificial
  Synthetic
cgctcagagggctgtctatc

SEQ ID NO: 147 - DNA, 20 bp, artificial
  Synthetic
tcgagcccaccgggaacgaa

SEQ ID NO: 148 - DNA, 20 bp, artificial
  Synthetic
gcaactggaccgaaggcgct

SEQ ID NO: 149 - DNA, 21 bp, artificial
  Synthetic
tggtagtagcaaccaacggga

SEQ ID NO: 150 - DNA, 22 bp, artificial
  Synthetic
actttgattgagggcgtcattc

SEQ ID NO: 151 - DNA, 23 bp, artificial
  Synthetic
atgatggcttattacagtggcaa

SEQ ID NO: 152 - DNA, 20 bp, artificial
  Synthetic
gtcggagattcgtagctgga

SEQ ID NO: 153 - DNA, 18 bp, artificial
  Synthetic
cctggtcaccagggctgc

SEQ ID NO: 154 - DNA, 19 bp, artificial
  Synthetic
ccgttctcagccttgacgg

(19) United States
(12) Patent Application Publication
(10) Pub. No.: US 2018/0251851 A1
(43) Pub. Date: Sep. 6, 2018

(54) ECTOPIC LYMPHOID STRUCTURES AS TARGETS FOR LIVER CANCER DETECTION, RISK PREDICTION AND THERAPY

(71) Applicants: Mathias HEIKENWALDER, Heidelberg (DE); YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Eli PIKARSKY, Jerusalem (IL); Yinon BEN-NERIAH, Mevasseret Zion (IL); Mathias HEIKENWALDER, Heidelberg (DE); Yujin HOSHIDA, Englewood Cliffs, NJ (US); Ilan STEIN, Tel-Aviv (IL); Shlomi FINKIN, New York, NY (US)

(21) Appl. No.: 15/759,219

(22) PCT Filed: Sep. 8, 2016
(86) PCT No.: PCT/IL2016/051002

§ 371 Date: Mar. 11, 2018

Related U.S. Application Data
(60) Provisional application No. 62/216,888, filed on Sep. 10, 2015.

(51) Int. Cl. C12Q 1/6886

(52) U.S. Cl. CPC C12Q 1/6886; C12Q 2600/158

(57) ABSTRACT

Methods of predicting the likelihood of cancer, or recurrence thereof, or determining eligibility for anti-cancer therapy, specifically liver cancer, are provided. Further, methods of determining liver ectopic lymphoid-like structures (ELS) as well as methods of treating liver cancer by disrupting liver ELS, are provided.

### CROSS REFERENCE TO RELATED APPLICATIONS

This application claims priority to U.S. Provisional Patent Application No. 62/216,888, filed Sep. 10, 2015, the contents of which are incorporated herein by reference in their entirety.

The research leading to these results has received funding from the European Research Council under the European Union's Seventh Framework Programme (FP7/2007-2013)/ERC grant agreement n° [281738,_20091118].

### FIELD OF INVENTION

The present invention is directed to the field of cancer diagnosis, prognosis and therapy, as well as identifying subjects suitable for specific anti-cancer treatments.

### BACKGROUND OF THE INVENTION

A central feature of tissue inflammation is the interaction between resident cells and immune cells. Cellular infiltration usually entails a diffuse influx of immune cells, scattered throughout the inflamed tissue. However, infiltrating leukocytes often form simple lymphoid aggregates or even more complex structures that histologically resemble lymphoid organs (Coppola D, et al. Am J Pathol 2011, 179(1): 37-45; Pitzalis C, et al. Nat Rev Immunol 2014, 14(7): 447-462; Dieu-Nosjean M C, et al. Trends in immunology 2014, 35(11): 571-580). These structures direct various B and T cell responses, possess organization of an appropriate microarchitecture and are referred to as ectopic lymphoid-like structures (ELS). ELSs often develop at sites of chronic inflammation where they influence the course of many diseases including distinct autoimmune, cardiovascular, metabolic and neurodegenerative diseases (Pitzalis C, 2014, ibid.).

Although the presence of ELSs within inflamed tissues has been linked to both protective and deleterious outcomes in patients, the mechanisms governing ectopic lymphoid neogenesis in human pathology remain poorly defined. In cancer, for example, solid tumors such as melanoma, colorectal and breast carcinoma, the presence of tumor-associated ELSs correlates with a better prognosis. In fact, the present literature in this respect unequivocally assigns an anti-tumor role for ELSs (Di Caro G, et al. Clinical cancer research: an official journal of the American Association for Cancer Research, 2014, 20(8): 2147-2158; Dieu-Nosjean M C, et al. Journal of clinical oncology: official journal of the American Society of Clinical Oncology, 2008, 26(27): 4410-4417; Gu-Trantien C, et al. J Clin Invest 2013, 123(7): 2873-2892; Messina J L, et al. Scientific reports 2012, 2: 765). It is believed that ELSs may coordinate endogenous antitumor immune responses that improve patient survival (Coppola D, 2011, ibid.; Dieu-Nosjean MC, 2014, ibid.; Gu-Trantien C, 2013, ibid.). A role for ELSs in the premalignant phase of tumor growth has not been explored so far.

Hepatocellular carcinoma (HCC) is a major health problem, being the second leading cause of cancer-related deaths worldwide. In most cases, human HCC is driven by chronic liver inflammation due to chronic viral hepatitis and non-alcoholic steatohepatitis (NASH) (El-Serag H B. The New England journal of medicine 2011, 365(12): 1118-1127; Umemura A, et al. Cell Metab 2014, 20(1): 133-144).

Formation of hepatic ELSs is a prominent pathological hallmark of chronic viral infection (Scheuer P J, et al. Hepatology 1992, 15(4): 567-571; Gerber M A. Clin Liver Dis 1997, 1(3): 529-541), yet a functional role for these immune follicles in HCC pathogenesis has not been suggested or explored.

### SUMMARY OF THE INVENTION

The present invention provides methods and kits for diagnosing, prognosticating and determining drug efficacy of specific types of solid cancers, particularly liver cancer. The present invention further provides compositions and methods for treating or ameliorating solid cancers associated with ectopic lymphoid-like structures (ELS), such as liver cancers.

The present invention is based, in part, on the surprising finding that hepatic ELS are indicative of hepatocellular carcinoma (HCC) as well as HCC recurrence. The present invention is further based, in part, on the surprising finding that treating hepatic ELS, such as by preventing their formation or disrupting their signaling, prevented HCC formation and growth thereof.

According to a first aspect, there is provided a method for predicting the likelihood of liver cancer or recurrence thereof in a subject in need thereof, the method comprising: determining at least one ELS-related parameter in the subject, wherein a parameter higher than a predefined control indicates the subject has a high likelihood of developing liver cancer.

According to another aspect, there is provided a method for determining eligibility for anti-cancer therapy in a subject having liver cancer, the method comprising: determining at least one ELS-related parameter in the subject, wherein a parameter higher than a predefined control is indicative that the subject is eligible for the anti-cancer therapy.

According to another embodiment, said anti-cancer therapy is surgical removal of the cancer. According to some embodiments, the anti-cancer therapy is an immunosuppressive drug. According to some embodiments, said immunosuppressive drug is selected from the group consisting of: glucocorticoids, cytostatics, or immunosuppressive antibodies. According to some embodiments, the anti-cancer therapy is an anti-ELS agent. According to some embodiments, the anti-ELS agent is selected from anti-CD90, LTβR-IG and CCR6 blockade.

According to some embodiments, a parameter higher than a predefined control is indicative that the subject is not suitable for an anti-cancer therapy selected from inhibitory immune checkpoint drugs.

Non-limiting examples of inhibitory immune checkpoint drugs are selected from drugs that inhibit one or more of the proteins selected from the group consisting of: A2AR, B7-H3, B7-H4, BTLA, CTLA-4, IDO, KIR, LAG3, PD-1, and TIM-3.

According to some embodiments, said determining at least one ELS-related parameter is determining binding of an ELS-binding agent. According to some embodiments, said ELS-binding agent is an agent specific for dendritic cells, high endothelial venules or other immune cells. According to some embodiments, said agent is an antibody against a surface receptor selected from the group consisting of: MECA-79, CD21, CD23, CD45, CD3, CD4, CD8, CD19, CD20, CD66b, CD14, CD33, and CD56.

According to some embodiments, said determining at least one ELS-related parameter is determining the presence or quantifying one or more ELS biomarkers. According to some embodiments, said one or more ELS biomarkers is selected from the group consisting of: LTβ, CCL17 and CCL20. According to some embodiments, said one or more ELS biomarkers is selected from the group consisting of: LTβ, CCL17, CCL20, CCL21, CCL19, CXCL13, CXCL11, CCL8, CXCL10, CXCL9, CCL2, CCL3, CCL18, CCL4 and CCL5. According to some embodiments, said ELS biomarkers is one or more biomarkers selected from LTβ, CCL17 and CCL20 and at least one additional biomarker selected from the group consisting of: LTβ, CCL17, CCL20, CCL21, CCL19, CXCL13, CXCL11, CCL8, CXCL10, CXCL9, CCL2, CCL3, CCL18, CCL4 and CCL5.

According to some embodiments, said one or more ELS biomarkers is a nucleic acid biomarker. According to some embodiments, said determining at least one ELS-related parameter is determining the presence or quantifying the expression levels of one or more ELS nucleic acid biomarkers.

According to some embodiments, said one or more ELS biomarkers is a protein biomarker. According to some embodiments, said determining at least one ELS-related parameter is determining the presence or quantifying the levels of one or more ELS protein biomarkers.

According to some embodiments, said determining at least one ELS-related parameter is histological determination of the presence of ELS.

According to some embodiments, said determining comprises: obtaining a biological sample from the subject; and determining at least one ELS-related parameter in said biological sample.

According to some embodiments, said biological sample is selected from the group consisting of: tissue, blood, serum, urine and cells. According to some embodiments, said biological sample is derived from a tumor, especially a liver biopsy.

According to some embodiments, said predefined control is selected from the group consisting of a non-cancerous sample from at least one individual, a panel of non-cancerous control samples from a set of individuals, and a stored set of data from control individuals.

According to another aspect, there is provided a method for determining the presence of ELS in the liver of a subject in need thereof, the method comprising:

(i) obtaining a liver sample from the subject; and

(ii) determining the expression of at least one biomarker selected from the group consisting of: LTβ, CCL17 and CCL20, in said sample,

wherein expression higher than a predefined control indicates the presence of ELS in the liver of said subject.

According to some embodiments, the method further comprises determining the expression of at least one additional biomarker selected from the group consisting of CCL21, CCL19, CXCL13, CXCL11, CCL8, CXCL10, CXCL9, CCL2, CCL3, CCL18, CCL4 and CCL5.

According to some embodiments, there is provided a method for predicting the likelihood of recurrent liver cancer in a subject after undergoing anti-cancer therapy, wherein a parameter higher than a predefined control is indicative that the subject has a high likelihood of late recurrence. According to a preferred embodiment the anti-cancer therapy is surgical removal of the cancer.

According to another aspect, there is provided a kit comprising one or more ligands, each ligand capable of specifically complexing with, binding to, hybridizing to, or quantitatively detecting or identifying an ELS-related parameter. According to some embodiments, said kit is for use in determining ELS in a liver sample. According to some embodiments, said one or more ligands are capable of specifically complexing with, binding to, hybridizing to, or quantitatively detecting or identifying a one or more biomarkers selected from the group consisting of: LTβ, CCL17, and CCL20. According to some embodiments, said one or more biomarkers are selected from the group consisting of: LTβ, CCL17, CCL20, CCL21, CCL19, CXCL13, CXCL11, CCL8, CXCL10, CXCL9, CCL2, CCL3, CCL18, CCL4 and CCL5.

According to another aspect, there is provided a method for treating or reducing the likelihood of liver cancer in a subject in need thereof, the method comprising administering to said subject a therapeutically effective amount of an anti-ELS agent, thereby treating or reducing the likelihood of liver cancer in said subject.

According to another aspect, there is provided a pharmaceutical composition comprising a therapeutically effective amount of one or more anti-ELS agents, for use treating or reducing the likelihood of liver cancer in a subject in need thereof.

According to another aspect, there is provided use of a pharmaceutical composition comprising a therapeutically effective amount of one or more anti-ELS agents, for the preparation of a medicament for treating or reducing the likelihood of liver cancer.

According to another aspect, there is provided a method for treating or reducing the likelihood of liver cancer in a subject in need thereof, the method comprising the steps of:

a. determining liver-ELS in the subject;

b. administering to said subject a therapeutically effective amount of anti-ELS agent, thereby treating or reducing the likelihood of HCC in said subject.

According to some embodiments, the anti-ELS agent is selected from the group consisting of anti-CD90 antibody, LTβR-IG or CCR6 blockade.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-F. Hepatic ELS signify a poor prognosis in human HCC and are associated with NF-κB activation. (1A) Upper panel, histological ELS score: For each human sample (n=82) the percentage of portal areas with ELS features (green) and the type of ELS was evaluated histologically (black and grey colors indicate presence and absence of histological ELS, respectively; Agg=Aggregate, Fol=Follicle, GC=Germinal Center). Gaps indicate lack of H&E-stained slides. Lower panel, ELS gene signature: Heatmap for expression of each of the 12 genes composing the ELS gene signature (Messina J L, et al. Scientific Reports 2012, 2: 765). Presence of high ELS gene signature is shown in the black color bar above the gene expression heatmap. Presence of high ELS gene signature was determined by coherent overexpression of the signature genes with statistical significance (prediction confidence p<0.05), as described in the Methods (black—present, grey—absent; upper horizontal bar). Cases in upper and lower panels are ordered according to ELS gene signature. (1B,C) Kaplan Meier curves for survival (1B) or late recurrence (1C) after resection of HCC, in patients with high and low ELS gene signatures in the liver parenchyma [n=82 patients (15 high score, 67 low score); *p=0.01 and 0.03 for (1B) and (1C), respectively, Log-rank test]. (1D,E) Hazard ratios of the ELS gene signature for overall survival (1D) and late recurrence (1E) in multivariable Cox regression modeling adjusted for 186-gene prognostic HCC risk and American Association for Study of Liver Diseases (AASLD) prognostic stage. Bars—95% confidence interval. (1F) Heatmap for NF-κB signature enrichment in the same cohort of human patients as in (1A). NF-κB signature enrichment was determined by modulation of 3 experimentally defined sets in HeLa cells, primary human fibroblasts and keratinocytes. In all 3 panels, samples are ordered according to the extent of ELS signature induction from left to right (same order as in 1A).

FIGS. 2A-E. Persistent liver IKK activation induces ectopic lymphoid structures. (2A) Immunoblot analysis for Flag-tagged IKKβ(EE) in tissues of Alb-cre control and IKKβ(EE)^{Hep} mice. Tubulin—loading control (shown two representative mice per group). (2B,C) Quantification of ELS number and size in IKKβ(EE)^{Hep} livers. Control Alb-cre mice do not develop follicles (n=10,8,6,5 for control, 4,7 and 14 months old IKKβ(EE)^{Hep} mice, respectively; *p=0.0002, **p=0.00001, two-tailed Students t-test, black line signifies mean). (2D) Representative H&E and immunostained sections of IKKβ(EE)^{Hep} mouse and human livers from patients with chronic hepatitis showing presence of immune follicles (scale bars 50 FDCs, follicular dendritic cells, HEVs, high endothelial venules). (2E) Cells from microscopically isolated ELSs from IKKβ(EE)^{Hep} mice livers were analyzed by flow cytometry for markers indicative of the shown cell types (black line signifies mean, results are representative of ELSs isolated from at least 6 IKKβ(EE)^{Hep} mice). Data are representative of three independent experiments in (2A) and (2D) and of one experiment in (2E).

FIGS. 3A-E. Persistent activation of IKK in hepatocytes induces aggressive HCC. (3A,B) Tumor number (≥0.5 cm) and volume in livers of 20-month-old Alb-cre control and IKKβ(EE)^{Hep} mice (n=13,11 for control and IKKβ(EE)^{Hep}, respectively; *p≤0.0002, two-tailed Students t-test, black line signifies mean). (3C) Representative livers and H&E stained sections from 20-month-old Alb-cre and IKKβ(EE)^{HeP} mice. Arrows indicate tumors (scale bar-200 μm). (3D) Representative H&E stains of HCCs from 20-month old IKKβ(EE)^{Hep} mice. WD-HCC=well-differentiated HCC, CCC=cholangiocellular carcinoma (scale bar—100 μm). (3E) Heat map representation of relative mRNA expression of a 16-gene HCC proliferation and differentiation signature (Cairo S, et al. Cancer Cell 2008, 14(6):471-484) in wild-type (WT) liver parenchyma or HCCs derived from the indicated mice. Clusters were determined by an unsupervised algorithm and designated A, B, the latter further subdivided into B1 and B2. Note that DEN induced HCCs from WT mice are more similar to WT liver parenchyma than IKKβ(EE)^{Hep}, most of which fall into cluster B together with the aggressive HCCs of Myc-TP53^{-/-} mice. Statistical analyses of tumor types in the different clusters: DEN WD-HCCs vs. all IKK HCCs (cluster A vs. B) p=6.0E-05; DEN WD vs. IKK DEN tumors (both WD and HCC-CCC tumors) (A vs. B) p=0.001; DEN WD-HCCs vs. IKK spontaneous (spon) HCCs (both WD-HCCs and HCC-CCC tumors) (A vs. B) p=0.04; DEN WD-HCCs-IKK HCC-CCC (A vs. B) p=0.006; IKK WD-HCCs vs. IKK HCC-CCC (B1 vs. B2) p=0.007. n=4,7,8,8,3 for WT, DEN treated Alb-cre, DEN treated IKKβ(EE)^{Hep}, untreated IKKβ(EE)^{Hep} and Myc-TP53^{-/-}mice, respectively; All p values were determined by two tailed chi-square test. Data are representative of two independent experiments in (A) and (B) and of one experiment in (E).

FIGS. 4A-F. HCC progenitors appear in ELSs and progressively egress out. (4A) Representative co-immunofluoresence stains for GFP (green) expressed from the hepatocyte specific IKKβ(EE) transgene and the epithelial marker E-cadherin (red) depicting the epithelial origin of HCC progenitors. Hoechst 33342 (blue) marks the nuclei (scale bars 100 μm). (4B) Representative immunostains for indicated HCC progenitor markers in ELSs of IKKβ(EE)^{Hep}mice (scale bars 50 μm). (4C) Representative H&E stained sections of IKKβ(EE)^{Hep} livers depicting ELS to HCC progression (arrow points to small ELS; scale bars 50 μm). (4D) Representative 3-dimensional (3D) reconstruction of an ELS from a 6-month old DEN-treated IKKβ(EE)^{Hep} mouse. Left upper panel: double color immunostaining for CD44v6 (brown) and B220 (red). Right upper panel: color conversion of the left panel (brown to green, red to purple). Lower panels: Two different rotations of a 3D reconstruction.

Note green CD44v6⁺ progenitor cells egressing out of the ELS at multiple points (scale bars 100 μm). α, β and γ show the same region in all panels. See also Supplementary video 1. (4E) Representative immunostains of two different livers (#1 and #2) from 14-month old untreated IKKβ(EE)^{Hep} mice and 6 month old DEN treated IKKβ(EE)^{Hep} mice for the pericentral marker glutamine synthetase (GS). Red and blue arrows indicate periportal and pericentral ELSs, respectively. Brown staining highlights pericentral zones (scale bars 100 μm). (4F) Representative confocal microscopy images of ELS-containing liver sections from a human patient for the HCC progenitor markers HSP70 (green) and Sox9 (purple) and for the bile duct marker CK19 (red). DAPI (blue) marks the nuclei. Arrow points to a group of HCC progenitors (scale bars 100 μm, arrow points to progenitor cell). Data are representative of three independent experiments in (A) and (B), and of one experiment in (D), (E) and (F).

FIG. 5A-G. Adaptive immune cells are required for ELS-dependent HCC promotion. (5A,B) Tumor numbers (≥3 mm and ≥5 mm, respectively) and volume (5C) in livers of 6 month old DEN-treated Alb-cre control, Rag1^{-/-}, IKKβ(EE)^{Hep} and IKKβ(EE)^{Hep}-Rag1^{-/-} (IKK-Rag) mice (n=11,10,12,12 for control, Rag1^{-/-}, IKKβ(EE)^{Hep} and IKK-Rag, respectively; *p≤0.006, two-tailed Students t-test; black line signifies mean). (5D) Representative images of livers from 6-month-old DEN-treated mice. Arrows indicate tumors. (5E,F) Tumor quantification by classification to well differentiated HCC (WD-HCC) or mixed cholangio-hepatocellular carcinoma (HCC-CCC). n=11 for each group; *p≤0.00004, two-tailed Students t-test; black line signifies mean. (5G) Representative immunostains for the HCC progenitor markers A6, CD44v6, CK19 and Sox9 in 6 month old DEN-treated livers. HCC progenitors in IKKβ(EE)^{Hep} liver are within ELSs, whereas the rare ones occasionally seen in IKKβ(EE)^{Hep}-Rag1^{-/-} mice are in the parenchyma (scale bars—50 μm). Data are representative of one experiment, n=8.

FIG. 6A-E. Anti-Thy1.2 immuno-ablative treatment during ELS development attenuates liver tumorigenesis. (6A) Representative images of immunostaining for CD3 in livers from control or anti-Thy1.2 injected 6 months old DEN-treated IKKβ(EE)^{Hep} mice (n=6, scale bars upper 200 μm, lower 50 μm). (6B) Mice were treated with control or anti Thy1.2 antibody. Representative sections from the entire liver were assessed for the total number of ELSs and presence of ELSs of various sizes as indicated (n=6,5 for control or anti-Thy1.2, respectively; *p≤0.04, **p≤0.003, black line signifies mean) (6C) Representative images of livers from control or anti-Thy1.2 injected 6-months-old DEN-treated IKKβ(EE)^{Hep} mice. n=6,10 for control and anti-Thy1.2, respectively; arrows indicate tumors. (6D,E) Tumor number (≥3 mm) (6D) and volume (6E) in livers of control or anti-Thy1.2 injected 6-month-old DEN-treated IKKβ(EE)^{Hep} mice (n=6,10 for control and anti-Thy1.2, respectively; *p≤0.04, two-tailed Students t-test, black line signifies mean). Data are representative of one experiment.

FIGS. 7A-H. ELS microniches provide a rich cytokine milieu. (7A) mRNA qPCR analysis of liver parenchyma and HCCs from IKKβ(EE)^{Hep} or DEN-treated IKKβ(EE)^{Hep} mice (M=months of age), as well as in liver parenchyma of 3-month old IKKβ(EE)^{Hep} mice without DEN treatment. Each data point reflects the median expression, normalized to the mean expression of the same gene in control livers derived from the equivalent Alb-cre control mice. (7B) Heat map (upper) and scatter plot (lower) representations of mRNA qPCR analyses of liver tissue from HCV-infected patients (n=43) relative to healthy controls (n=12). Scatter plots depict mRNA amounts of LTβ, CCL17 and CCL20; *p<0.0001, two-tailed Students t-test, Log₁₀ scale, cross line signifies mean). (7C) Representative immunostaining for LTβ in HCV-infected human liver (scale bars: upper 200 μm; lower 50 μm). (7D) Representative LTβ-mRNA in situhybridization in mouse livers (scale bars 50 μm). (7E) Quantification of LTβ expression in malignant hepatocytes. The % of LTβ positive hepatocytes was determined by counting 10 ELSs from each mouse (n=8,5,6,5 for control, 3, 6 and 9 months old mice, respectively; *p=0.0003, **p=0.00006, two-tailed Students t-test, black line signifies mean). (7F) Representative serial sections showing LTβ mRNA in-situ hybridization and immunostaining for the progenitor marker A6. Note LTβ staining of immune cells and egressing hepatocytes (black arrows) but not niche residing ones (white arrows, scale bars-100 μm). (7G) Representative images of LTβ-mRNA in-situ hybridization in hepatic ELSs of control or anti-Thy1.2 injected 6 month old DEN-treated IKKβ(EE)^{Hep} mice (scale bars-50 μm). (7H) qPCR analysis of LTβ mRNA expression in liver parenchyma of control or anti-Thy1.2 injected 6 month old DEN-treated IKKβ(EE)^{Hep} mice (n=10,6 respectively; *p=0.003 two-tailed Students t-test, black line signifies mean). Data are representative of one experiment in (A) and (B) and of two independent experiments in (C), (D), (F) and (G).

FIGS. 8A-G. Blocking LT signaling abolishes microniche egression and tumorigenesis. (8A) Heat map representation of mRNA qPCR analysis of liver parenchyma from 33-weeks-old IKKβ(EE)^{Hep} mice treated with LTβR-Ig for 10 consecutive weeks (23-32 weeks). Each data point reflects the median expression, normalized to the mean expression of the same gene in equivalent control murine-IgG1-injected IKKβ(EE)^{Hep} mice (Log₂ scale).(8B) Tumor number (≥0.5 cm) in livers of 33 week old IKKβ(EE)^{Hep} mice treated with either control-Ig or LTμR-Ig for the indicated periods (n=12,11,10,11 for control, 3-12w, 13-22w or 23-32w, respectively; *p=0.04, **p=0.0002, two-tailed Students t-test, black line signifies mean).(8C,D) Quantification of the percent of ELSs showing egressed progenitor hepatocytes (8C) and of the number of egressing hepatocyte clusters per ELS (8D) [n=7,11 for control-Ig and LTμR-Ig treated mice, respectively; *p=0.02, and 0.00009 for (C) and (D), respectively; two-tailed Students t-test, black line signifies mean]. (8E) Quantification of the CDC47⁺Sox9⁺ double positive cells in ELSs (see below, pink in G, right panels) (n=6; *p=0.02, two-tailed Students t-test, black line signifies mean). (8F) Quantification of the GFP⁺ cells inside the ELSs (n=6; *p=0.001, two-tailed Students t-test, black line signifies mean). (8G) Representative confocal microscopy images of ELS-containing liver sections from DEN-treated IKKβ(EE)^{Hep} mice injected for 10 consecutive weeks (23-32 weeks) with control-Ig or LTβR-Ig for GFP, CDC47 and Sox9. Hoechst 33342 marks the nuclei. Arrows indicate CDC47⁺Sox9⁺ double positive cells in pink (scale bars 100 μm). Data are representative of one experiment in (A) and (B) and of two independent experiments in (G).

FIGS. 9A-F. Hepatic ELSs signify a poor prognosis in human HCC and are associated with NF-κB activation. (9A) Intrahepatic ELSs in human livers were classified as vague follicular aggregates (Agg), definite round-shaped clusters of small lymphocytes without germinal center (Fol), and follicles with definite germinal centers composed of large lymphocytes with clear cytoplasm (GC) according to published criteria. (9B-D) Kaplan-Meier curves for probability of early (9B) or late (9C) recurrence or of overall survival (9D) after resection of HCC, in patients with high and low ELS histological score in the liver parenchyma [66 patients with H&E staining out of 82 patients (14 high score, 52 low score); p as indicated, log-rank test]. In 9B black arrow denotes the end of the high ELS score line. (9E) Kaplan Meier curves for probability of early recurrence after resection of HCC, in patients with high and low ELS scores in the liver parenchyma [n=82 patients (15 high score, 67 low score); n.s.—not significant p=0.78, 0.04, 0.18 and 0.34 for b-e, respectively, Log-rank test]. (9F) Gene set enrichment index assessing correlation between enrichment of 3 different published sets of NF-κB targets (X axis) and histological ELS score in human livers (Y axis) [n=66 patients (14 high score, 52 low score); p as indicated, two-tailed Students t-test].

FIGS. 10A-O. Constitutive activation of the NF-icB pathway in hepatocytes induces mild liver inflammation. (10A) NF-κB DNA binding was analyzed by EMSA in nuclear extracts from Alb-cre control, IKKβ(EE)^{Hep}, TNF-treated Alb-cre for 30 or 60 minutes and Mdr2^{-/-} mice. To examine the composition of NF-κB dimers, Alb-cre-TNFtreated nuclear extract was supershifted with RelA/p65 antibody. One of two Mdr2^{-/-} lanes was removed from the figure for esthetic reasons. (10B) Quantification of EMSA. Results are representative of three independent experiments (*p<0.05, two-tailed Students t-test, bars—mean±SEM). (10C) qPCR analysis for TNF and KC of mice described in a (control, IKKβ(EE)^{Hep}, Mdr2^{-/-}, TNF-treated Alb-cre 30′/60′: n=7,5,6,4,4, respectively; *p=0.01, **p=0.0002; bars—mean±SEM). (10D) Representative immunohistochemical stains for GFP and RelA/p65 of livers from 6 months old Alb-cre control and IKKβ(EE)^{Hep} mice (scale bars-25 μm). (1E) Representative H&E stains of liver tissue from 3 months-old Alb-cre control and IKKβ(EE)^{Hep} mice (scale bars: upper panels-200 μm, lower panels-50 μm). (10F) Representative photomicrographs of F4/80-stained liver sections from 7 months old Alb-cre control and IKKβ(EE)^{Hep} mice (scale bars: 50 μm upper panels, 25 μm lower panels). (10G) Quantification of F4/80⁺ cells (expressed as % of total) shown in F(control: n=11, IKKβ(EE)^{Hep}: n=6, *p=0.002, two-tailed Students t-test, bars—mean±SEM). (10H,I) Alanine transaminase (ALT) and aspartate aminotransferase (AST) levels were measured in sera of 7 months old Alb-cre control and IKKβ(EE)^{Hep} mice (control: n=8, IKKβ(EE)^{H}eP: n=5, *p<0.01, two-tailed Students t-test, bars—mean±SEM). Normal range of ALT and AST: 17-77 and 54-191 U/L, respectively. Note that while AST levels in IKKβ(EE)^{Hep} mice are significantly higher than in controls they are still within the normal range. (10J) Representative Ki67 immunostains of Alb-cre control and IKKβ(EE)^{Hep} mice liver parenchyma at the indicated ages (scale bars-50 μm). (10K) Quantification of Ki67 immunostains described in J (n=6,5,7,7 for 1,4,7,20 months control mice, respectively; n=5,4,7,7 for 1,4,7,20 months IKKβ(EE)^{Hep} mice, respectively; n.s—non significant, *p=0.03, **p=1E-07, two-tailed Students t-test, bars—mean±SEM; hpf—high power field). (10L,M) Splenocytes of 5-months-old Alb-cre mice (I, upper panels) or from microscopically isolated ELSs from 5 months old DEN treated IKKβ(EE)^{Hep} mice livers (L lower panels and M) were analyzed by flow cytometry for the following cell surface markers: CD4, CD8, CD44 and CD62L in 1; CD45, CD11b⁺, MHCII⁺ (as a marker for activation) and F4/80⁺ in m. For both l and m results are representative of ELSs isolated from 6 IKKβ(EE)^{Hep} mice (bars—mean±SEM). (10N) Representative co-immunofluorescence stains for B and T lymphocytes in IKKβ(EE)^{Hep} mice (top) and human (bottom) ELSs, showing clear compartmentalization. CD3 labels T cells and B220/CD20 labels B cells. DAPI (blue) marks the nuclei (scale bars-100 μm). (100) mRNA qPCR analysis for the ELS gene signature in liver parenchyma from Alb-cre control and IKKREEPP mice at the indicated ages (n=12,7,11 for control, 14 and 20 months old IKKβ(EE)^{Hep}, respectively; *p<0.05, **p<0.01, ***p<0.001, ****p<0.0001, two-tailed Students t-test, bars—mean±SEM. For each gene the three columns correspond to control, 14 months IKKβ(EE)^{Hep}, and 20 months IKKβ(EE)^{Hep}. Data are representative of one experiment except for (d), (f), (j), (n) and (o) which are representative of two independent experiments.

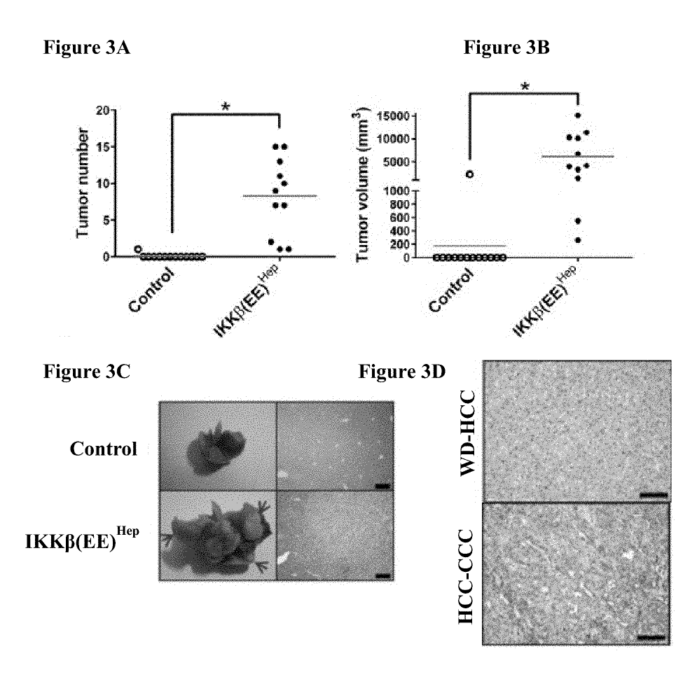

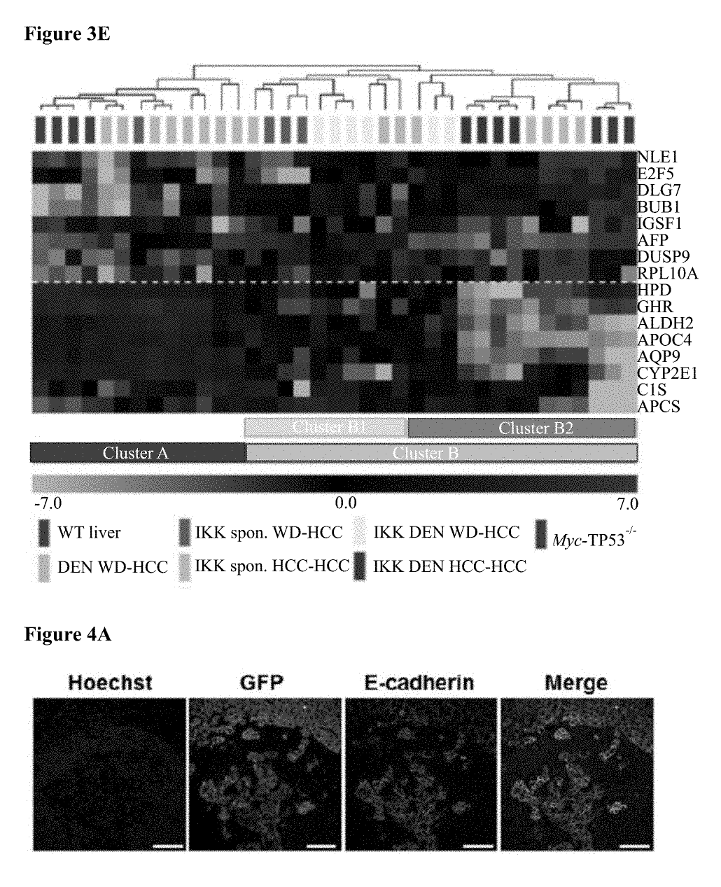

FIG. 11A-T. IKKβ(EE) expression in hepatocytes induces HCC and metastases. (11A) Representative immunostains for A6, glutamine synthetase (GS) and Ki67 in tumors of IKKβ(EE)^{Hep} mice (scale bars-50 μm). (11B) Immunostains for collagen IV of liver tissue from untreated Alb-cre or tumors from 9-months old DEN-treated Alb-cre control mouse and 20-months old IKKβ(EE)^{Hep} mouse. Parenchyma (P) and Tumor (T) areas are indicated; Red dashed line depicts P/T border (-50 μm). (11C) Representative H&E stained sections showing lymph node and lung metastasis in 20 months old IKKβ(EE)^{Hep} mice (scale bars: left panels 500 —μm, right panels 50 μm). (11D,E) ELS number (D) and diameter (E) in livers of Alb-cre control, IKKβ(+/E)^{Hep} hemizygotes and IKKREEPP homozygotes mice at the indicated ages. Control Alb-cre mice do not develop ELSs (n=10,8,6,5,4 for control, 4,7 and 14 months old IKKβ(EE)^{Hep} or IKKβ(+/E)^{Hep} mice, respectively; *p<0.002, **p<0.0003, two-tailed Students t-test, bars—mean±SEM). (11F,G) Tumor number (F, >0.5 cm) and volume (G) in livers of 20-month-old Alb-cre control, IKKβ(+/E)^{Hep} hemizygotes and IKKREEPP homozygotes mice (n=13,9,11 for control, IKKβ(+/E)^{Hep} and IKKβ(EE)^{Hep} mice, respectively; n.s.=not significant, *p<0.05, **p<0.0001, two-tailed Students t-test, black cross line signifies mean). Data regarding Control and IKKβ(EE)^{Hep} homozygotes mice are the same as in FIG. 3a-b and are shown here as a reference. (11H) Representative photomicrographs of F4/80-stained liver sections from 9 months-old control and Alb-IKKβ(EE) mice (scale bars, upper panel-50 lower panel—25 μm). (11!) H&E stains of liver tissue from 9 months-old Alb-IKKβ(EE) mice reveal the presence of ELSs (scale bars-50 μm). (11J) Liver and H&E stained section of liver tissue from 12-month-old Alb-IKKβ(EE) mouse. Arrows indicate tumors on the liver surface (scale bar-50 μm). (11K) Representative H&E stained sections of livers from DEN-treated IKKβ(EE)^{Hep} mice at the indicated ages (scale bars-upper panels: 200 μm, lower panels: 50 μm). (11L,M) Quantification of ELSs number/cm² and diameter (μm) in untreated and DEN-treated IKKβ(EE)^{Hep} mice. Alb-cre control mice, either untreated or DEN-treated, do not develop ELSs (n=5/5,8/9,8/9,6/10 for control, 3,6 and 9 months old untreated or DEN-treated IKKβ(EE)^{Hep} mice, respectively; *p=0.001, **p<0.0001 for (L) and *p<0.0001 for (M), two-tailed Students t-test, mean±SEM). (11N,O) Tumor (≥0.5 cm) number and total volume in livers of 9-month-old DEN-treated Alb-cre control and IKKβ(EE)^{Hep} mice (n=12, 8, 11 for DEN-treated control, untreated IKKβ(EE)^{Hep} and DEN-treated IKKβ(EE)^{Hep} mice, respectively; *p<0.001 for (N) and *p<0.01 for (O), two-tailed Students t-test, black cross line signifies mean). (P) Representative livers and H&E stained sections of liver tissue from 9-month-old DEN-treated Alb-cre control and IKKβ(EE)^{Hep} mice. Arrows and dashed lines indicate tumors (scale bars-200 μm). (Q) Representative Ki-67 immunostains of DEN-treated Alb-cre control and IKKβ(EE)^{Hep} mice liver parenchyma or tumor (mon-months, scale bars-50 μm). (R) Quantification of Ki67⁺ hepatocytes in liver parenchyma (par) and tumors of DEN-treated Alb-cre control and IKKβ(EE)^{Hep} mice (n=5,6,5,5,7,8,4,4,7,8 for the indicated mice, respectively; *p<0.01, **p<0.001, two-tailed Students t-test, bars—mean±SEM). (S) Aberration score for each of the tumors analyzed by aCGH [Data are stored and available from ArrayExpress (https://www.ebi.ac.uk/arrayexpress/) accession number E-MTAB-3848]. Score (0-3) was determined based on size and number of aberrations in each tumor. Well differentiated tumors (WD-HCC) were compared to mixed (HCC-CCC) tumors in each group (n=6,7,7 for control, IKKβ(EE)^{Hep}+DEN and IKKβ(EE)^{Hep} mice, respectively; *p<0.05, two-tailed Students t-test, bars—mean±SEM). (T) Genomic DNA was extracted from parenchyma of Alb-cre control mice or from tumors of IKKβ(EE)^{Hep} mice and subjected to copy number variation analysis by digital PCR. Rgs2 and Gab2 are genes located at the centers of two chromosomal regions found to be amplified in aCGH analysis of HCCs from IKKβ(EE)^{Hep} mice (see s). Dashed lines depict average plus 2 standard deviations of the control group for Rgs2 and Gab2, respectively. Tert—a reference for unamplified DNA (n=11,13 for control and IKKβ(EE)^{Hep}, respectively). Tert, Rgs2 and Gab2 are given in that order for each sample. Data are representative of one experiment except (L), (M), (N) and (Q), which are representative of two independent experiments.

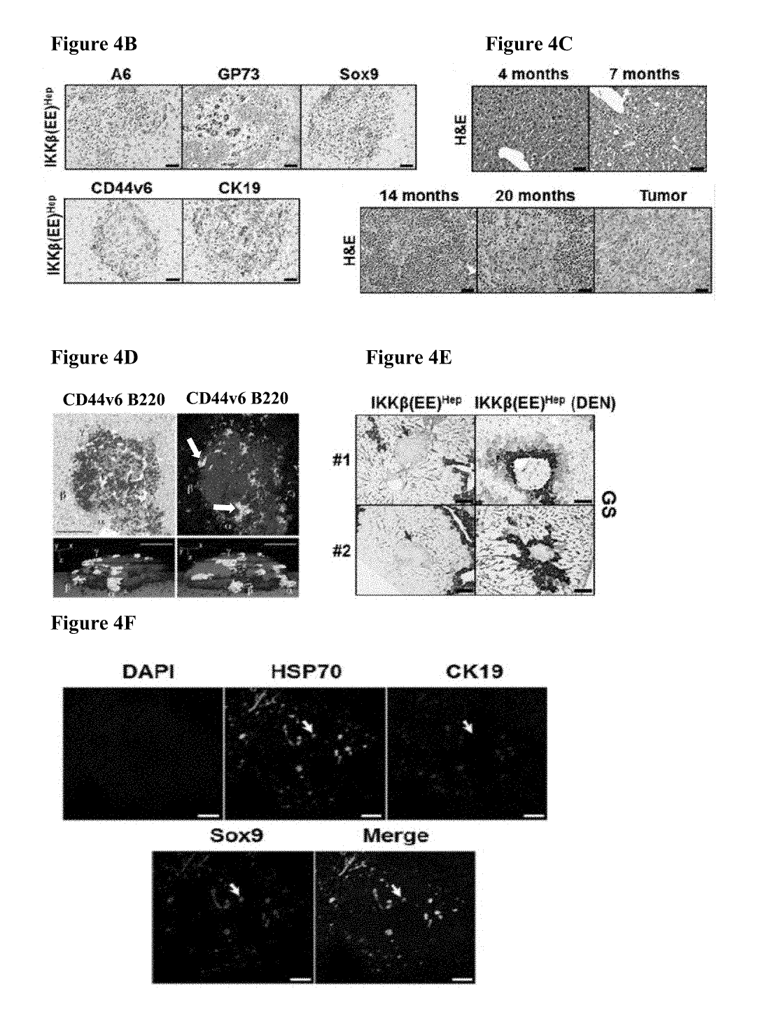

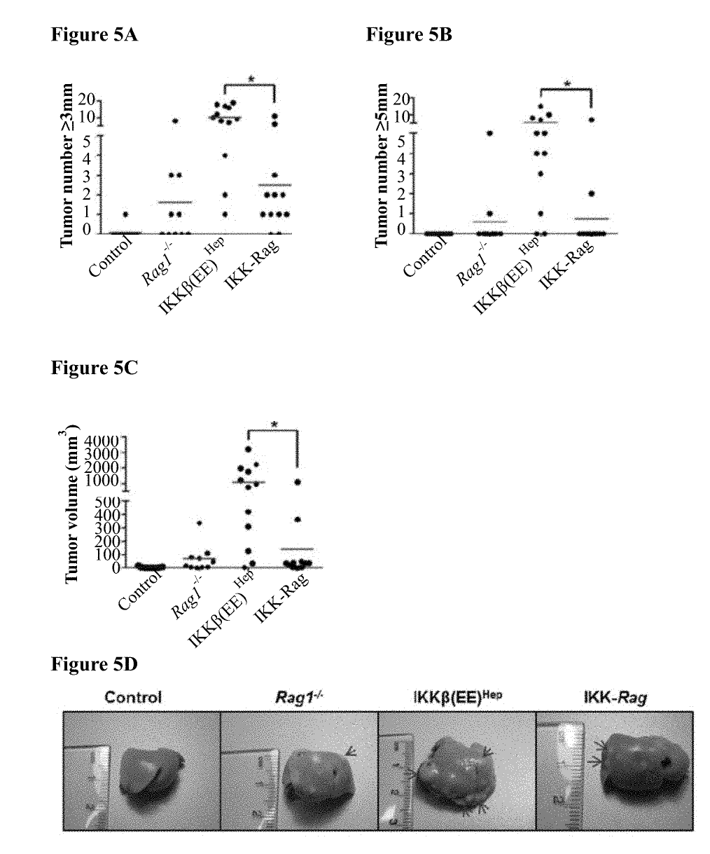

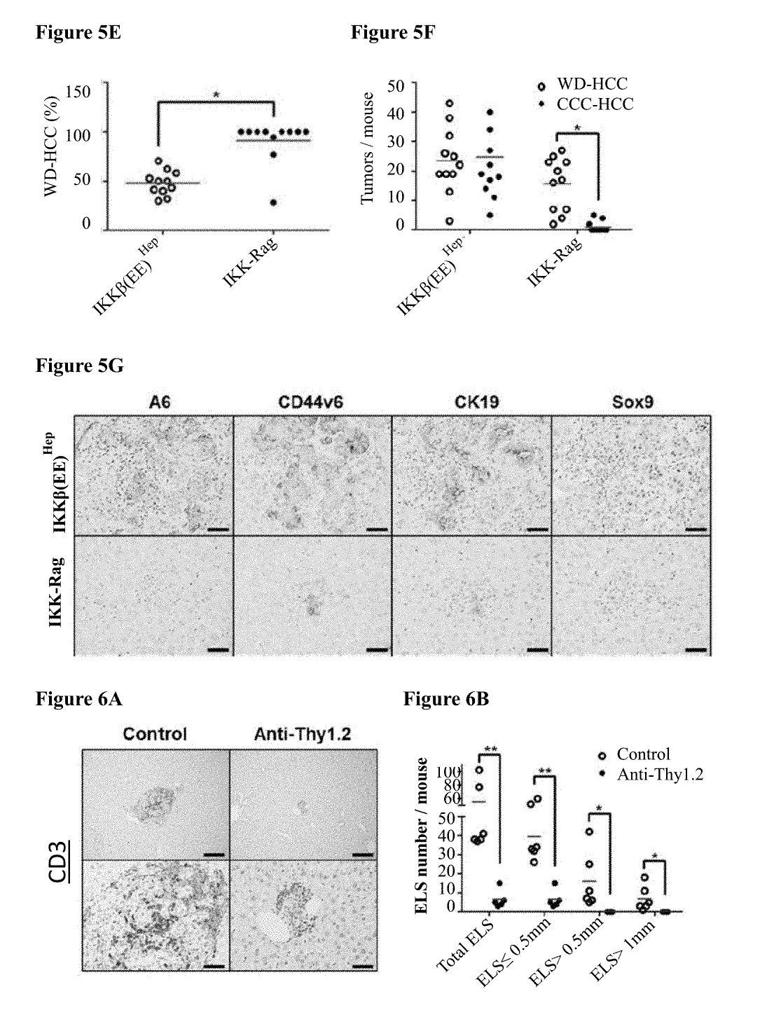

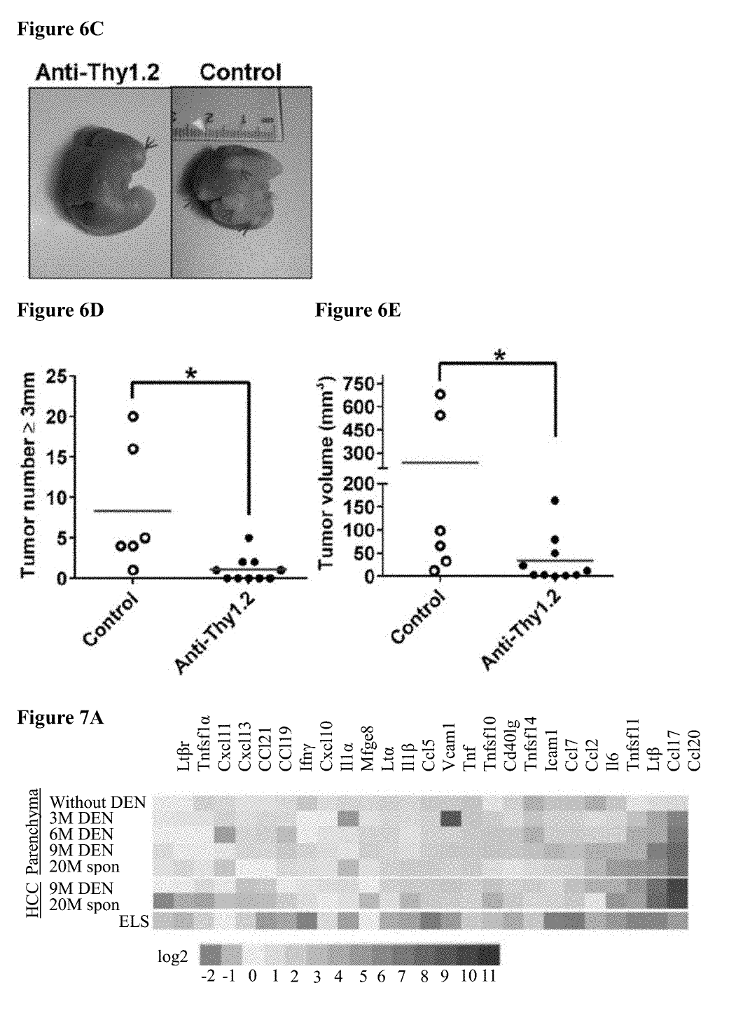

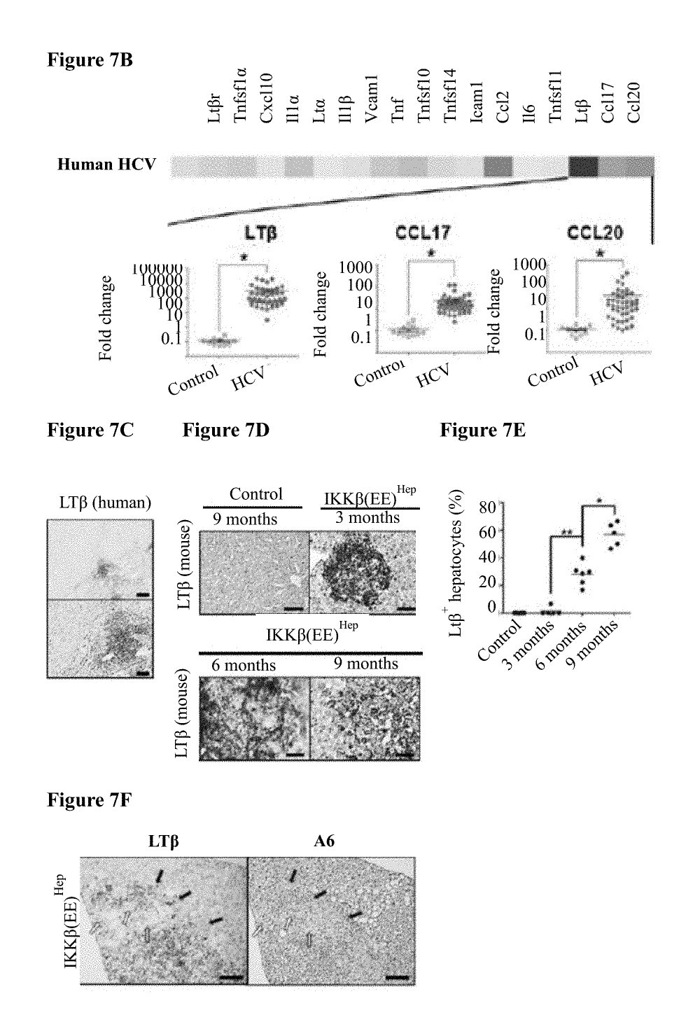

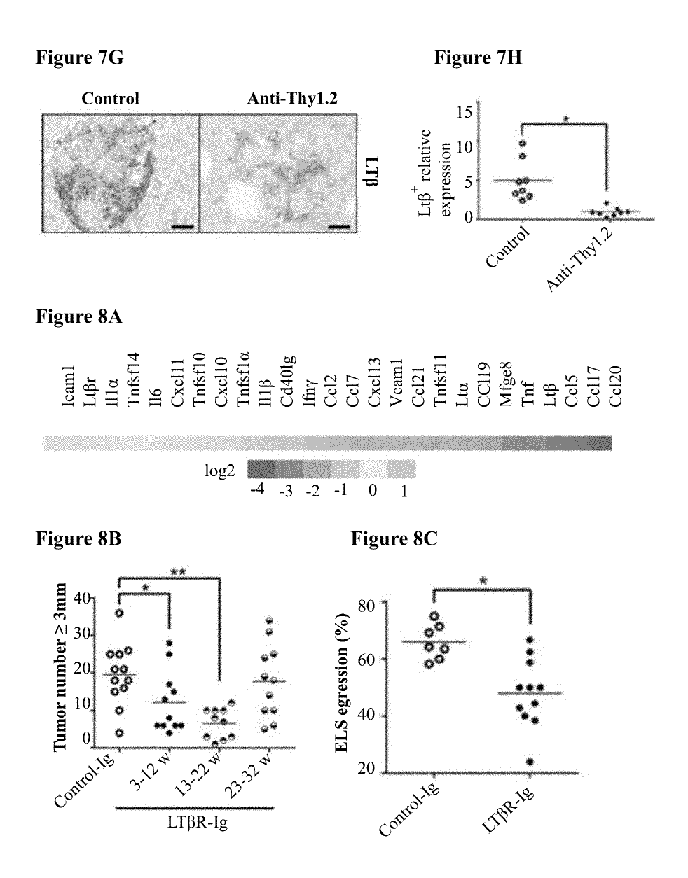

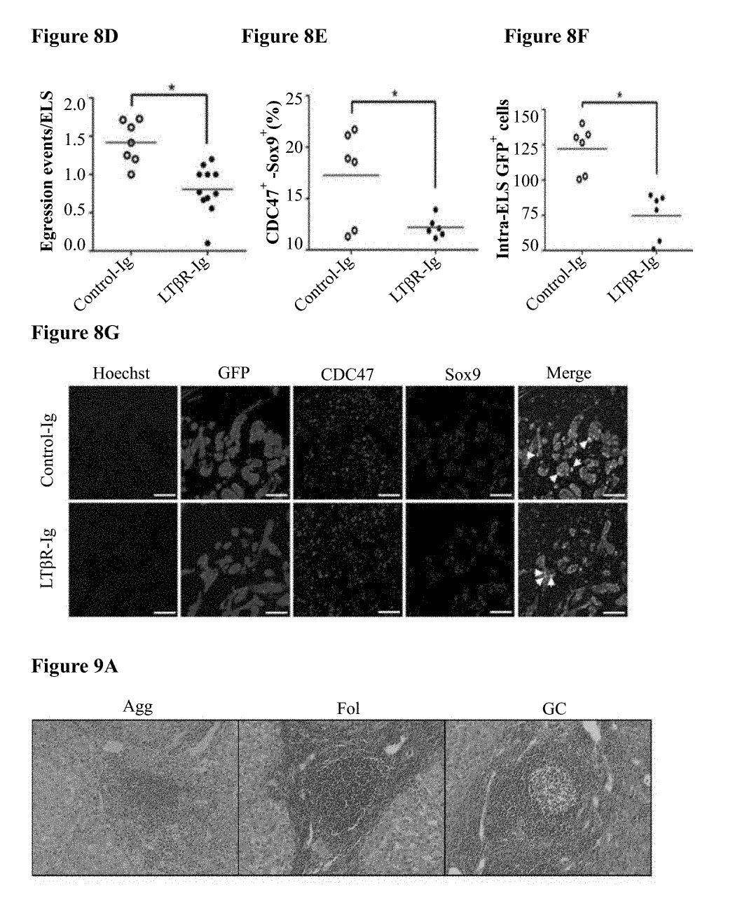

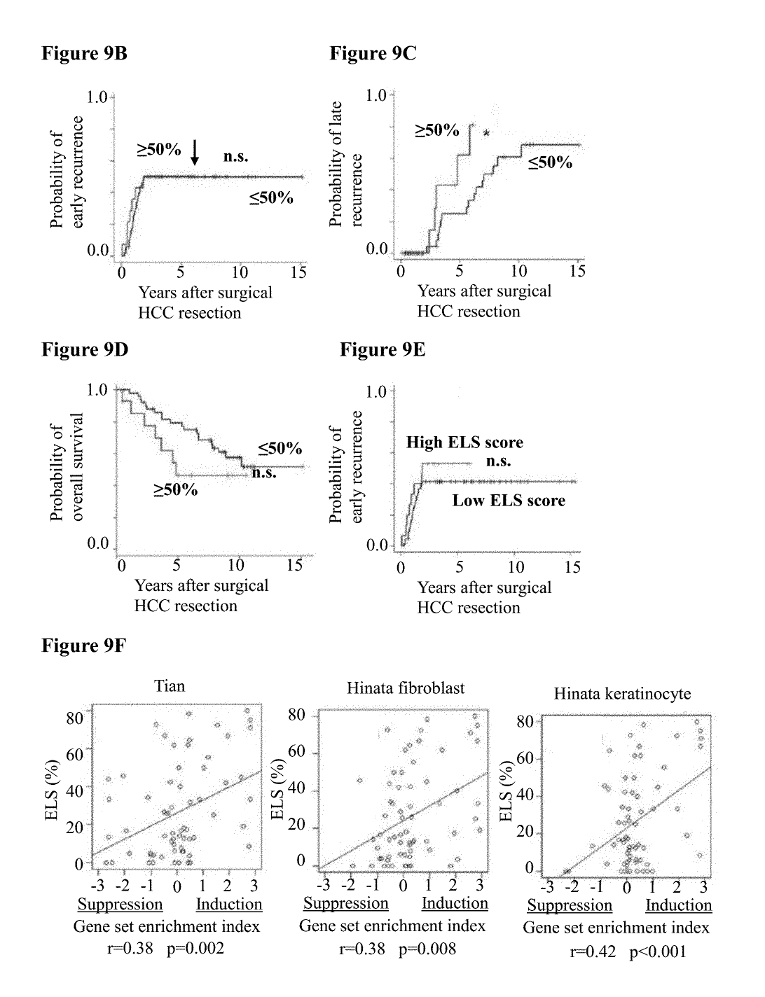

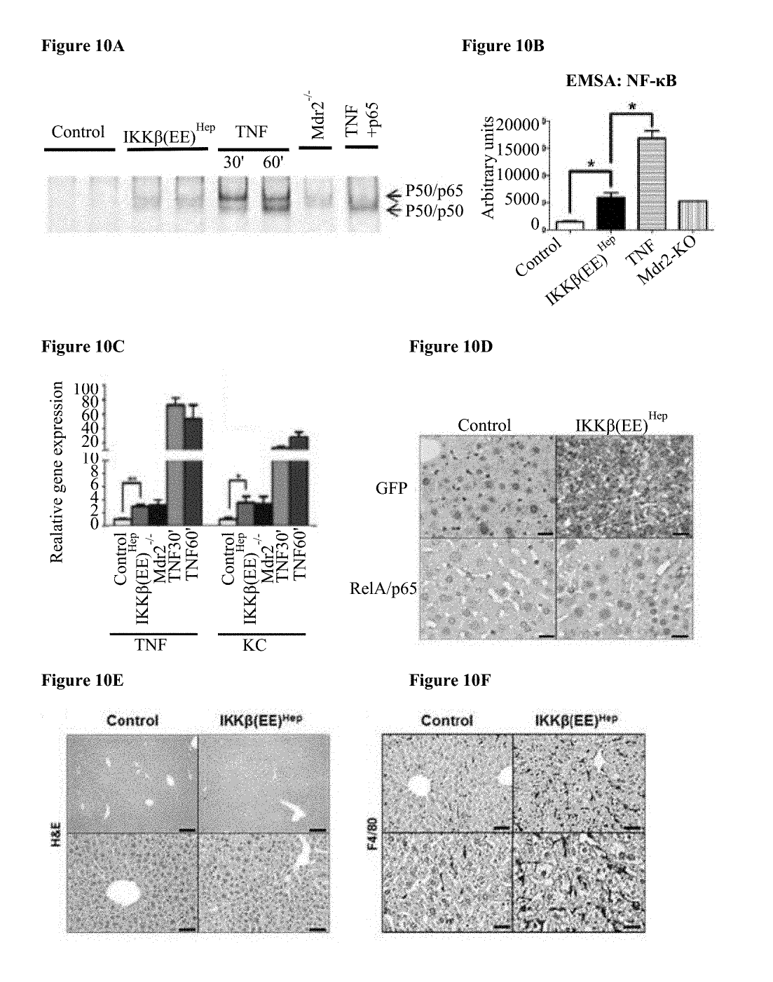

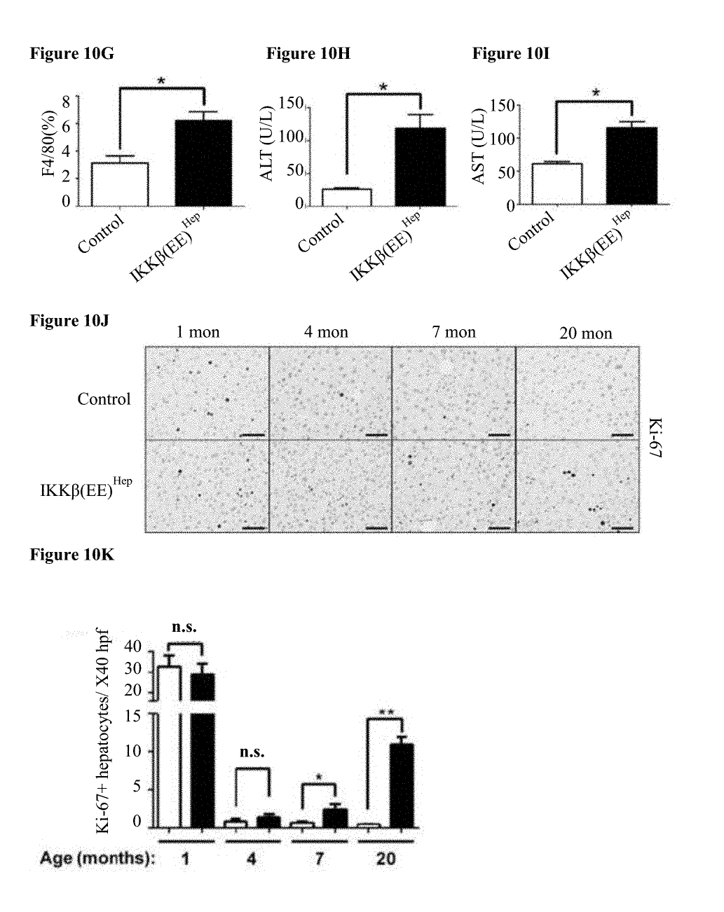

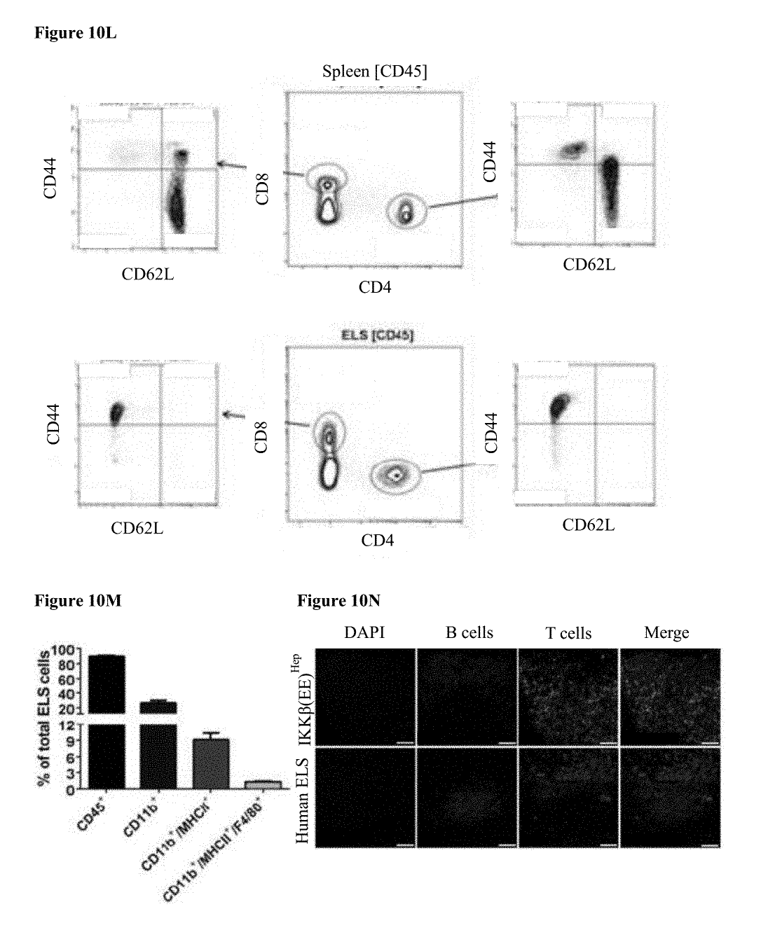

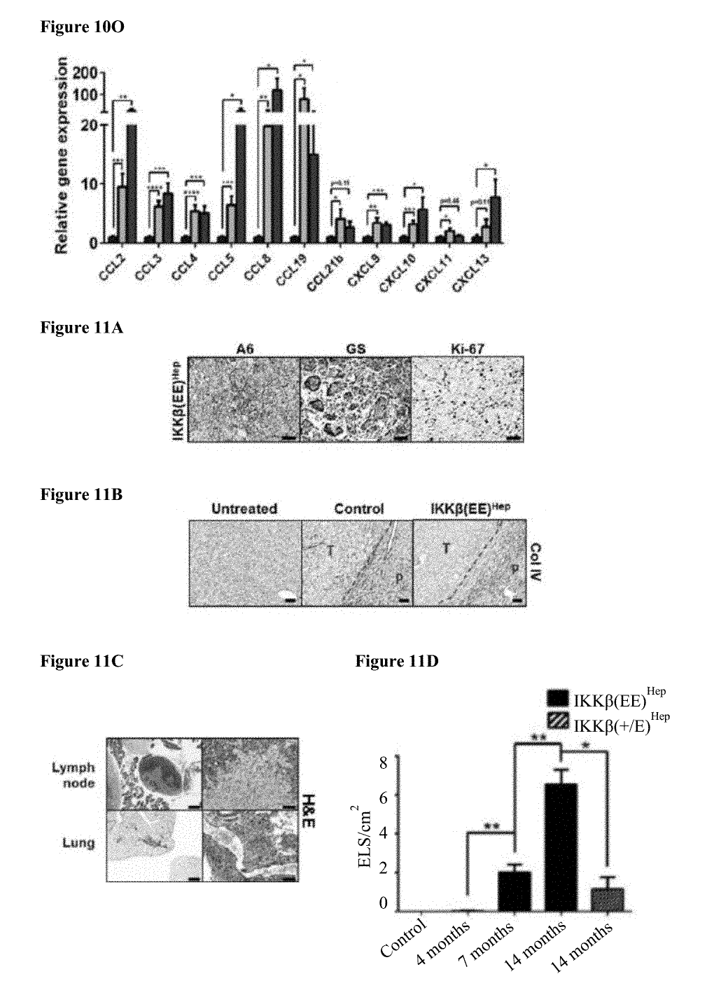

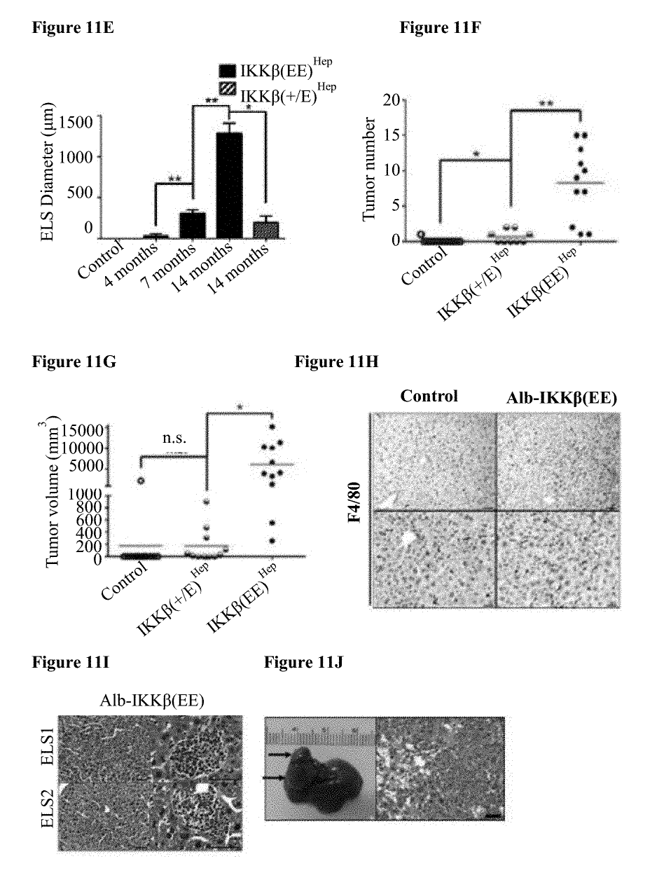

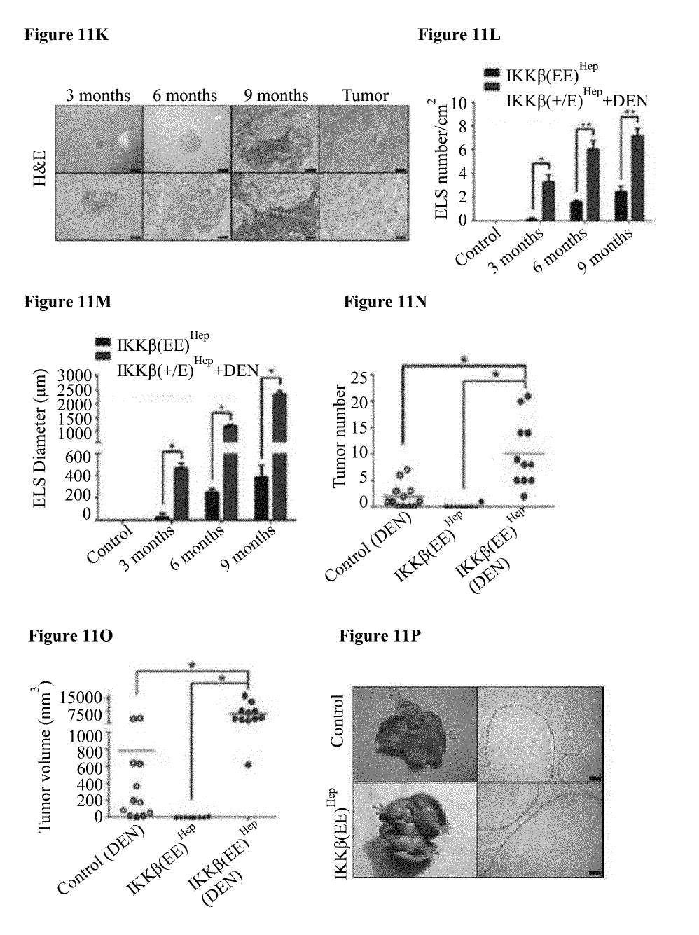

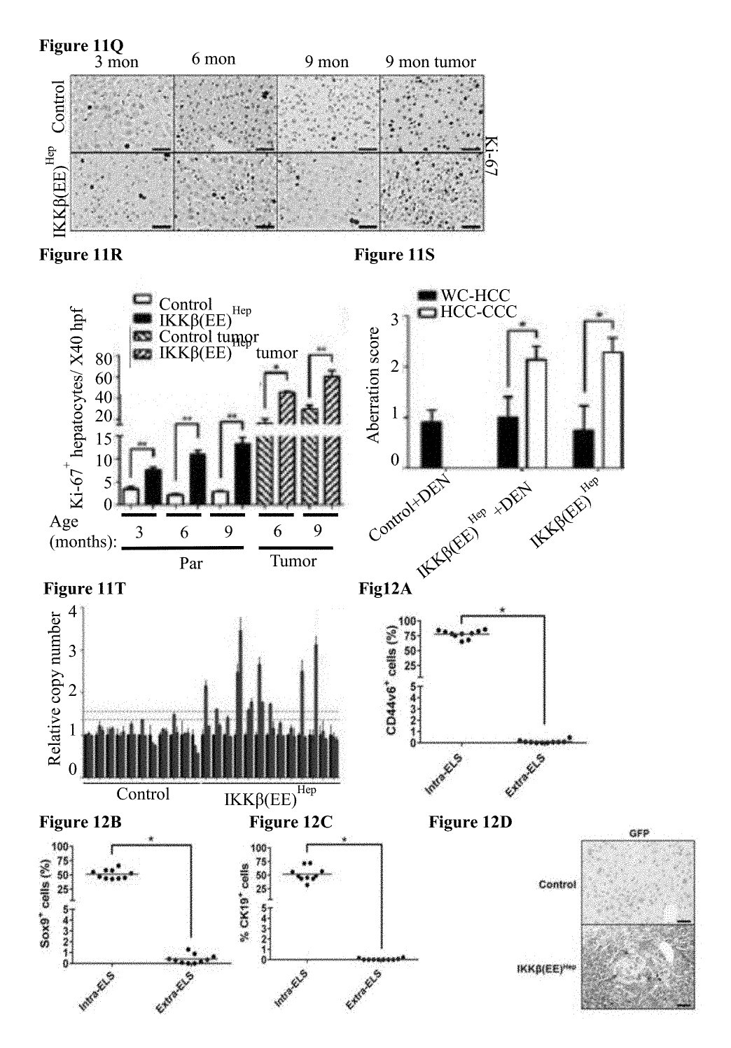

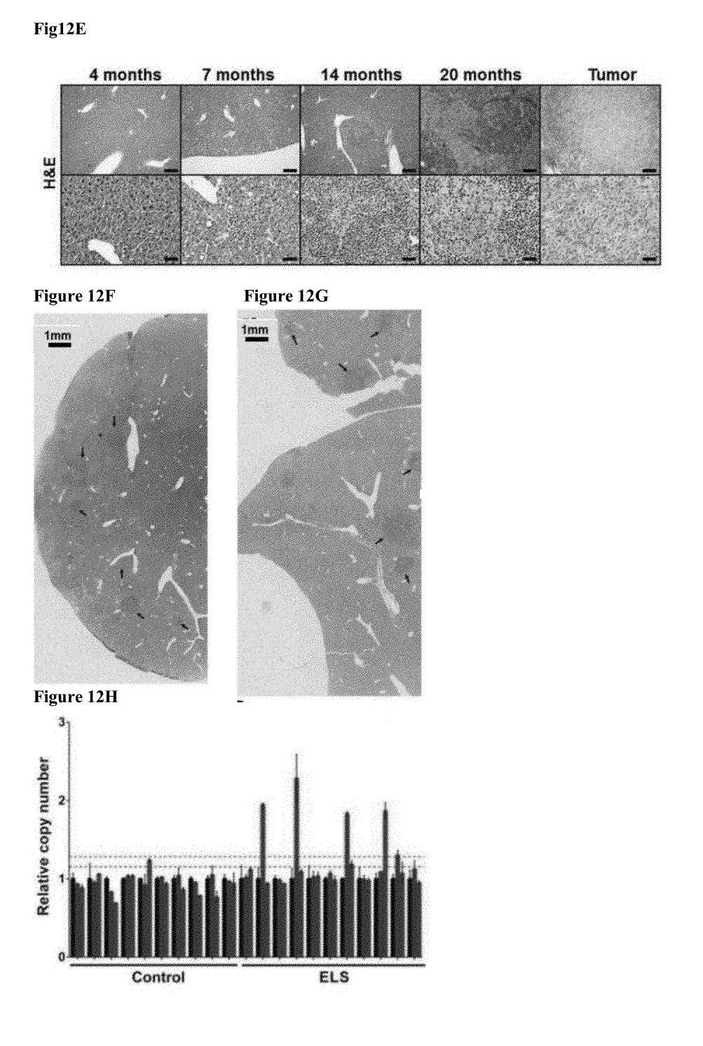

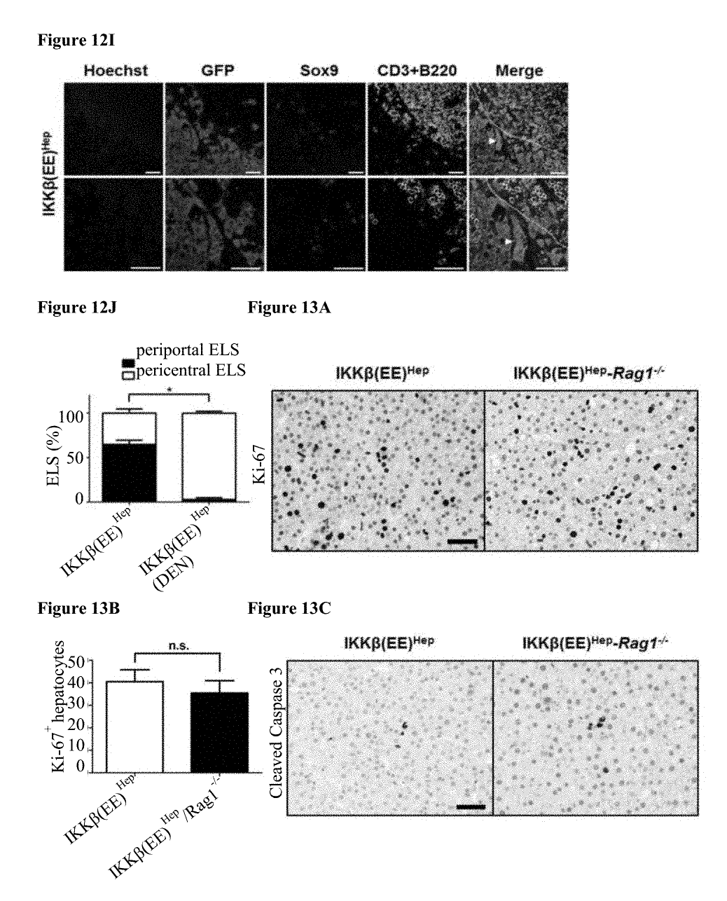

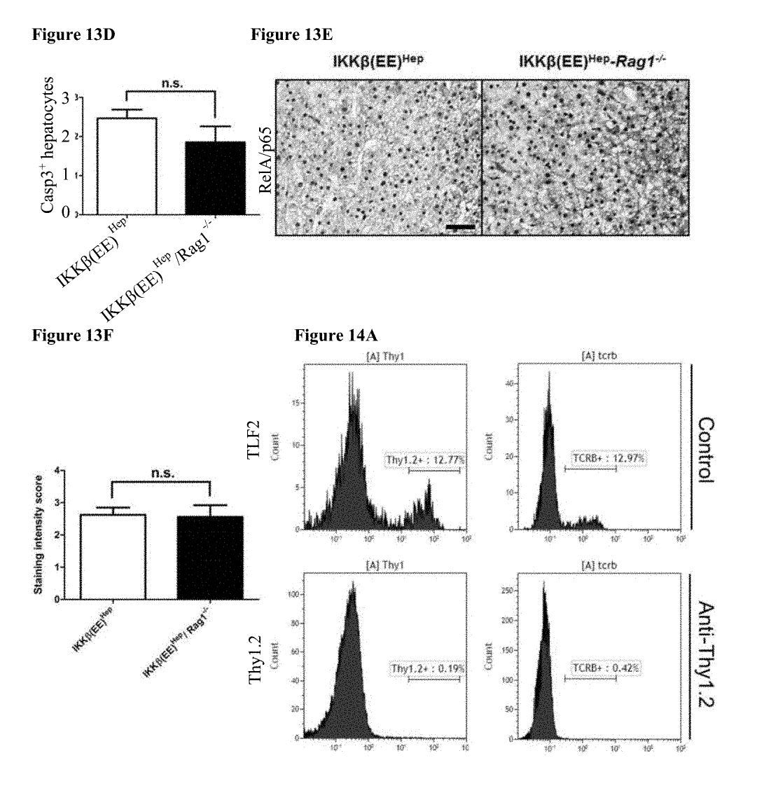

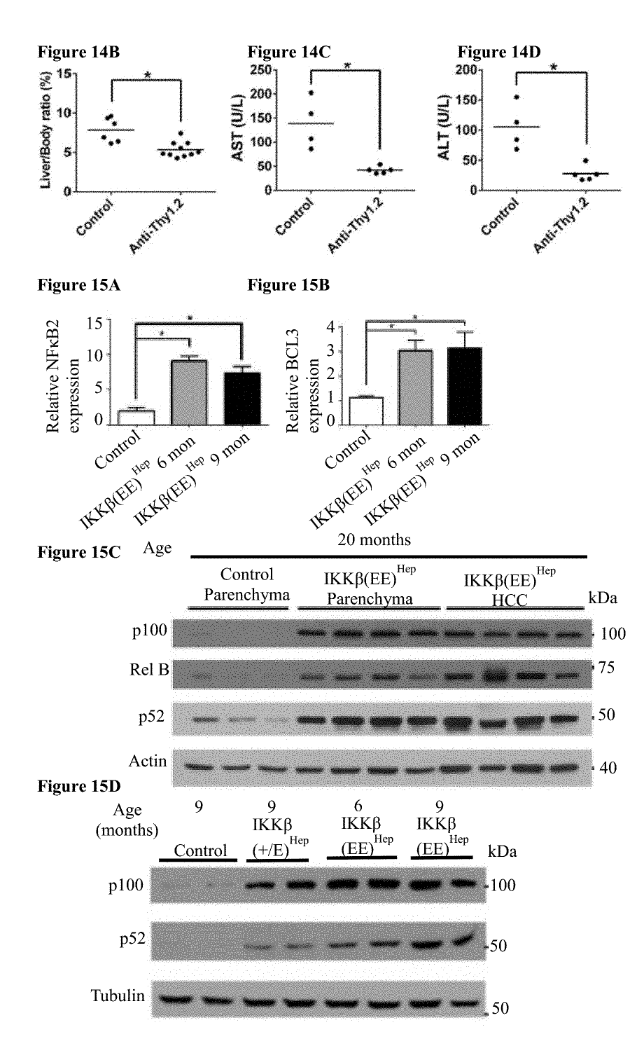

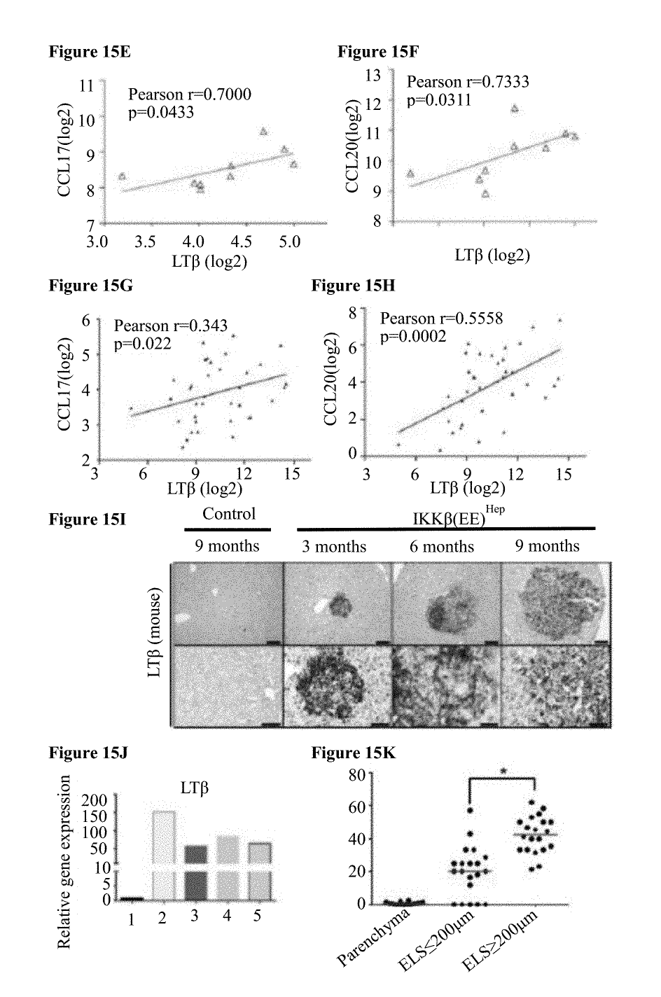

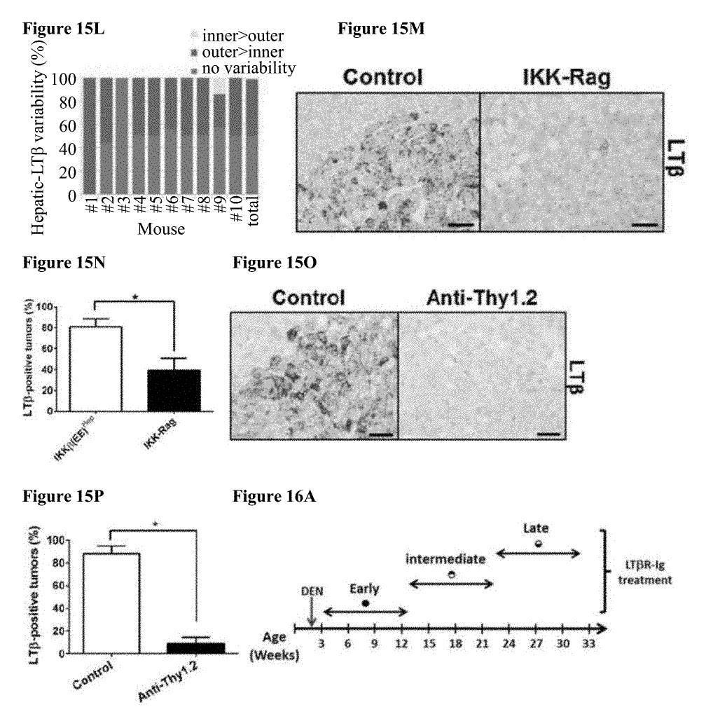

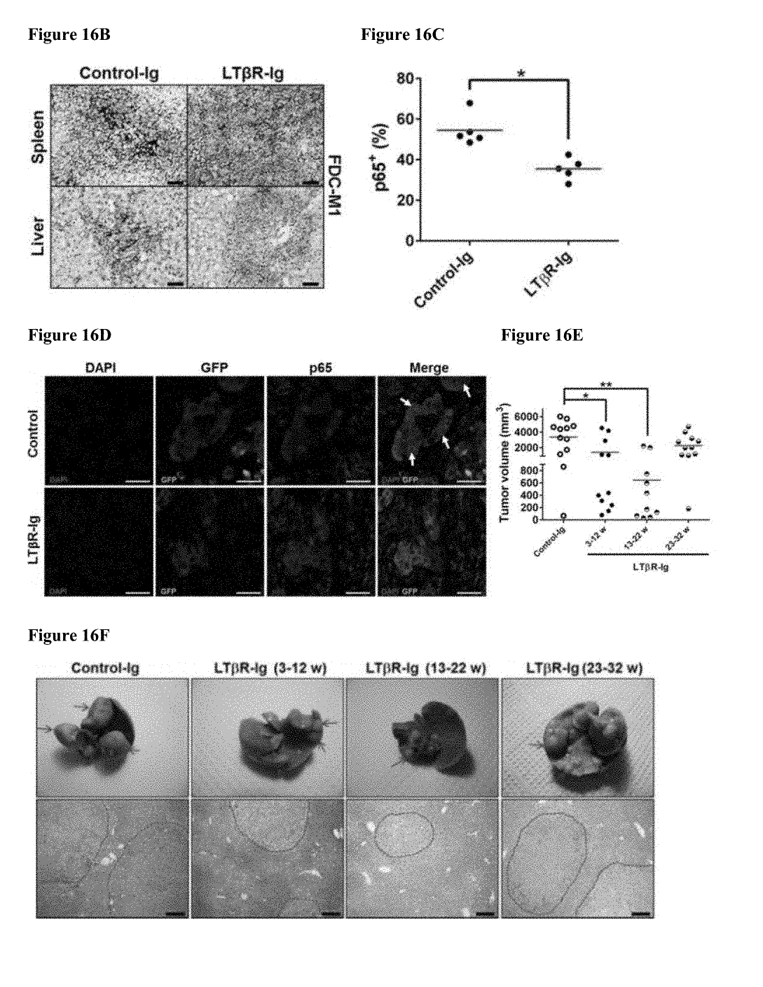

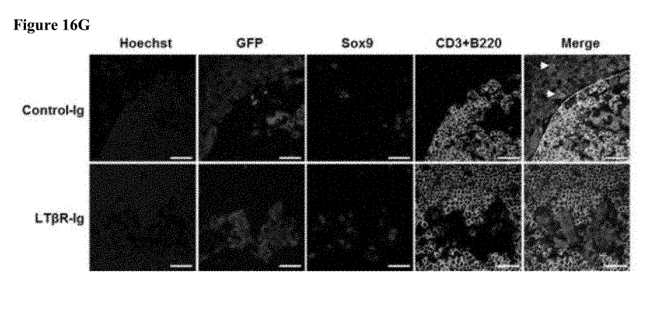

FIG. 12A-J. Tumor progenitor cells gradually egress out from their supportive microniche. (12A-C) Consecutive liver sections were immune-stained for the HCC progenitor markers CD44v6, Sox9 and CK19 as indicated. The percent of positive cells (out of total hepatocytes) within ELS and in liver parenchyma is shown (n=10, *P≤1.6E-10, two-tailed Students t-test, bars—mean±SEM). (12D) Representative immunostains for GFP (scale bars-50 μm). (12E) Representative H&E stained sections of IKKβ(EE)^{Hep} livers depicting ELS to HCC progression (arrows point to small ELSs; scale bars, upper panels-200 μm, lower panels-50 μm). Lower panels are the same ones shown in FIG. 4C. (12F,G) Representative whole slide scans of H&E stained sections from 20 months old IKKβ(EE)^{H}eP liver (F, and 9 months old IKKβ(EE)^{Hep} DEN-treated liver (G). Note that in each liver multiple ELSs and tumors at various stages of progression are present (arrows indicate ELSs, scale bars-1 mm). (12H) Genomic DNA, extracted from parenchyma or from tumor progenitors cells isolated by laser capture micro-dissection from ELSs of 5 months old IKKβ(EE)^{Hep} livers, was subjected to copy number variation analysis by digital PCR. Rgs2 and Gab2 are genes located at the centers of two chromosomal regions found to be amplified in aCGH analysis of HCCs from IKKβ(EE)^{Hep} mice (see FIG. 11T) Tert, Rgs2 and Gab2 are given in that order for each sample. Dashed lines depict average plus 2 standard deviations of the control group for Rgs2 and Gab2, respectively. Tert used as a reference for unamplified region (n=10,11 for parenchymal and ELS derived hepatocytes, respectively). (12I) Representative co-immunofluorescence stains depicting the ELS egression process. Lymphocytes (CD3+B220) highlight ELS border. GFP labels all hepatocytes, Sox9 labels malignant hepatocytes. Hoechst 33342 (blue) marks the nuclei. Arrows points to egressing cluster (scale bars-100 μm). (12J) Quantification of ELS zonation in livers of 14-20-months-old untreated IKKβ(EE)^{Hep} mice and 6 months old DEN treated IKKβ(EE)^{Hep} mice (n=9, *p=2.1E-09, two-tailed Students t-test, bars—mean±SEM). Data are representative of one experiment except for (D) and (J) which are representative of two independent experiments.

FIG. 13A-F. Proliferation, apoptosis and NF-KB activation do not differ between well differentiated HCCs from IKKβ(EE)^{Hep} and IKKβ(EE)^{Hep}-Rag1^{-/-} mice. Representative immunostains for the proliferation marker Ki67 (13A), apoptosis marker Cleaved Caspase 3 (1B) and RelA/p65 (13C) in livers of 6 months old IKKβ(EE)^{Hep} and Rag1^{-/-}-IKKβ(EE)^{Hep} mice (scale bars'50 μm). Graphs on the right (13D,E,F, respectively) depict quantification of the corresponding immunostain. Ki-67 and cleaved Caspase 3 positive hepatocytes were counted in 10, arbitrary chosen, high power fields; RelA/p65 stain was quantified using an arbitrary subjective scoring scale (0-4) of nuclear staining (n=5, n.s—not significant, two-tailed Students t-test, bars—mean±SEM).

FIG. 14A-D. Anti-Thy1.2 treatment during ELS development attenuates liver tumorigenesis. (14A) Blood samples were taken from TLF2 (isotype control) injected or from anti-Thy1.2 injected 6 months old DEN-treated IKKβ(EE)^{Hep} mice and analyzed by flow cytometry for two T cell markers, Thyl and TCRP. Numbers above the bars represent percentage of positively stained cells. (14B) Liver to body ratio of control or anti-Thy1.2 treated 6 months old DEN-injected IKKβ(EE)^{Hep} mice (n=6,10 for control and anti-Thy1.2 treated mice, respectively; *p<0.01, two-tailed Students t-test, bars—mean±SEM). (14C,D) AST and ALT levels were measured in sera of control or anti-Thy1.2 treated 6 months old DEN-injected IKKβ(EE)^{Hep} mice (n=5, *p<0.01, two-tailed Students t-test, bars—mean±SEM).

FIG. 15A-P. Activation of LT pathway in IKKβ(EE)^{Hep} mice and HCV infected patients. (15A,B) qPCR analysis of mRNA levels of NF-κB2 and Bcl3, respectively, in DEN-treated Alb-cre control and IKKβ(EE)^{Hep} mice at the indicated ages (n=5,5,6, respectively; *p<0.001, two-tailed Students t-test, bars—mean±SEM). (15C) Immunoblot analysis of protein extracts from either liver parenchyma or HCCs of 20-months old Alb-cre control and IKKβ(EE)^{Hep} mice for p100, RelB and p52. Actin-loading control. (15D) Immunoblot analysis of protein extracts from livers of DEN-treated Alb-cre control, IKKβ(+/E)^{Hep} and IKKβ(EE)^{Hep} mice at the indicated ages for p100 and p52. Tubulin loading control. (15E,F) Spearman correlation plots of mRNA expression levels of LTβ vs. CCL17 and LTβ vs. CCL20, respectively, in ELSs dissected from 6 months old DEN-treated IKKβ(EE)^{Hep} mice (n=9). 15G,H) Pearson correlation plots of mRNA expression levels of LTβ vs. CCL17 and LTβ vs. CCL20, respectively, in human HCV infected livers (n=43). (15I) Representative LTβ-mRNA in-situ hybridization in mouse livers (scale bars-100 μm upper panels, 50 μm lower panels). (15J) Cells from microscopically isolated ELSs from IKKβ(EE)^{Hep} mice livers were FACS-sorted for the shown cell types and then analyzed for LTβ expression by real time PCR. Hep (par)=parenchymal hepatocytes from IKKβ(EE)^{Hep} mice livers. Results are representative of ELSs isolated from 3 IKKβ(EE)^{Hep} mice. 1=Hep (par), 2=B cells, 3=Tₕ cells, 4=T_{c} cells, 5=Other leukocytes. (15K) Quantification of the percentage of LTβ positive progenitor malignant hepatocytes in parenchyma, small (≤200 μm) and large (>200 μm) ELSs (10 sections containing ELSs from IKKβ(EE)^{Hep} mice were quantified, *p<0.0001, two-tailed Students t-test, black cross line signifies mean). (15L) Quantification of tumor progenitor LTβ variability. LTβ mRNA expression in ELS associated hepatocytes was assessed in 10 individual IKKβ(EE)^{Hep} mice (labeled #1 to #10). For each mouse, all ELSs in a single liver section were scored. Each ELS was scored as either “inner>outer” where stronger staining was noted in hepatocytes in the ELS core compared with the periphery (yellow); “no variability” indicating no apparent difference in LTβ hepatocyte expression (blue); “outer>inner”—higher expression in hepatocytes in the ELS periphery (red). Total: cumulative score of all assessed ELSs: 26 out of 54 ELSs were scored as “outer>inner” vs. 1 out of 54 that was scored as “inner>outer”, p=0.0001, Fisher exact test. (15M,N) Representative images (M) and quantification (N) of LTβ-mRNA in-situ hybridization in liver tumors of 6-month-old DEN-treated IKKβ(EE)^{Hep} mice or IKKβ(EE)^{Hep}-Rag1^{-/-} (IKK-Rag) mice (scale bars-50 μm, n=5, *p<0.0001, two-tailed Students t-test, bars—mean±SEM). (15O,P) Representative images (O) and quantification (P) of LTβ-mRNA in-situ hybridization in liver ELSs and tumors of 6-month-old DEN-injected IKKβ(EE)^{Hep} mice treated with either isotype control antibody (LTF2) or anti-Thy1.2 antibody (scale bars—50 μm, n=5,4, respectively; *p<0.0001, two-tailed Students t-test, bars—mean±SEM).

FIG. 16A-G. Blocking LT signaling abolishes microniche egression and tumorigenesis. (16A) Schematic representation of long term LTβR-Ig treatment. IKKβ(EE)^{Hep} mice were given a single injection of DEN at 15 days of age, followed by 3 different regimens of 10 weeks LTβR-Ig or control-Ig administration (100 μg per week), as indicated. All mice were sacrificed at 33 weeks of age. (16B)

Representative immunostains for FDC-M1 of spleen and liver sections from DEN-treated IKKβ(EE)^{Hep} mice injected for 10 consecutive weeks (13-22 weeks, see a for details) with control-Ig or LTβR-Ig and sacrificed three days after the last injection (scale bars-50 μm). (16C,D) Quantification (C) and representative high power confocal microscopy images (D) for p65 (red) staining in control or LTβR-Ig treated IKKβ(EE)^{Hep} mice. GFP (green) marks hepatocytes, DAPI (blue) marks the nuclei (*p=0.002, arrows point to nuclear p65 in intra-ELS HCC progenitors, scale bars-50 μm). (E) Tumor volume of livers of 33 weeks old IKKβ(EE)^{Hep} mice treated with either control-Ig or LTβR-Ig for the indicated periods (n=12,11,10,11 for control-Ig, 3-12W, 13-23W, 23-32W, respectively; *p=0.02, **p<0.0007, two-tailed Students t-test, red cross line signifies mean). (F) Representative images of whole livers and H&E stained sections. Arrows and dashed lines indicate visible tumors on the liver surface and H&E stained sections, respectively (scale bars-200 μm). (G) High power confocal microscopy images: GFP (green) marks all hepatocytes, Sox9 (red) marks progenitor hepatocytes, B220+CD3 (white) mark lymphocytes and Hoechst 33342 (blue) marks the nuclei. Arrows point to egressing malignant hepatocytes (scale bars-upper panels-100 μm, lower panels-50 μm).

### DETAILED DESCRIPTION OF THE INVENTION

The present invention provides methods and kits for solid cancer, in particular liver cancer, detection, assessment and therapy.

By one aspect, the present invention concerns a method for predicting the likelihood of cancer in a subject at risk for developing cancer, the method comprising: determining at least one ELS-related parameter in the subject, a parameter higher than a predefined control indicating the subject has a high likelihood of developing cancer.

By another aspect, the present invention concerns a method for predicting cancer reoccurrence in a subject, after undergoing anti-cancer therapy, the method comprising: determining at least one ELS-related parameter in the subject, a parameter higher than a predefined control indicating the subject has a high likelihood of having cancer reoccurrence.

By another aspect, the present invention concerns a method for determining ELS in a liver, or vicinity thereof, of a subject in need thereof, the method comprising: obtaining a biological sample from the subject; and determining at least one ELS-related parameter in said biological sample, wherein a parameter higher than a predefined control is indicative that the subject has ELS is the liver.

The term “cancer” refers to any type of cancer and in particular cancers other than melanoma, colorectal carcinoma and breast carcinoma. In some embodiments, the cancer is a solid tumor other than melanoma, colorectal carcinoma and breast carcinoma. In exemplary embodiments, the cancer is liver cancer. In some embodiments, the cancer is primary liver cancer, including but not limited to, hepatocellular carcinoma (HCC), fibrolamellar carcinoma, cholangiocarcinoma, hepatoblastoma, angiosarcoma, or cystadenocarcinoma. In exemplary embodiments, the cancer is HCC.

The term “subject at risk of developing cancer” includes but is not limited to a subject that due to genetic disposition, exposure to environmental substances, or a pre-existing non-cancer pathological condition, is at a higher risk than the general population to develop cancer later in life and thus should be routinely monitored. In embodiments where the subject has a high risk of developing HCC, the pre-existing non-cancer pathological condition, may be chronic liver inflammation. In some embodiments, the non-cancer pathological condition is selected from fibrotic/cirrhotic liver conditions, for example due to chronic viral hepatitis (e.g., HBV or HCV) and NASH. According to some embodiments, the pre-existing non-cancer pathological condition is associated with a condition selected from non-alcoholic fatty liver diseases (NAFLD) and non-alcoholic steatohepatitis (NASH). According to some embodiments, the pre-existing non-cancer pathological condition is associated with alcoholic liver disease. Additional non-limiting examples of pre-existing non-cancer pathological conditions are hereditary hemochromatosis, alpha-one-antitrypsin deficiency, auto-immune hepatitis, some porphyrias or Wilson's disease

As used herein, the term “diagnosis” means detecting a disease or disorder or determining the stage, severity or degree of a disease or disorder, distinguishing a disease from other diseases including those diseases that may feature one or more similar or identical symptoms, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder, e.g., HCC.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrases “prognosticating” and “determining the prognosis” are used interchangeably and refer to the process by which the skilled artisan can predict the course or outcome of a condition in a patient. The skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely. The term “long-term” survival is used herein to refer to survival for at least 5 years, for at least 8 years, or for at least 10 years following surgery or other treatment.

The term “cancer reoccurrence” refers to appearance of cancer in a subject undergoing and/or after anti-cancer therapy, and includes early or late reoccurrence of cancer.

As used herein, the term “time to recurrence” or “TTR” is used herein to refer to time in years to first recurrence censoring for second primary cancer as a first event or death without evidence of recurrence.

Non-limiting examples of anti-cancer therapy include anti-cancer treatment by drugs, radiation or surgery. In exemplary embodiments, the anti-cancer therapy is surgical removal of the solid tumor, such as HCC resection. In some embodiments, the subject has previously undergone surgical removal of a solid tumor, such as HCC resection. In accordance with some embodiments of the present invention, the predication is of late reoccurrence of the cancer (e.g., HCC) after resection, such as after more than 2 years from the resection.

As exemplified herein, and without wishing to be bound by any mechanism or theory, late recurrence of HCC (e.g., later than 2 years following resection) indicates de novo generation of HCC, the prediction is that presence of an ELS indicator in the liver of a subject with chronic liver inflammation forecasts the chances of developing first time HCC.

The term “ELS-related parameter” refers to any parameter that indicates the presence of ELSs in the tested subject or sample derived from the tested subject. None limiting examples of ELS-related parameters include imaging assays for ELS detection, and detection of the presence of one or more biomarkers, such as protein and/or nucleic acid biomarkers.

In some embodiments, the presence of ELS is determined by detecting or determining the presence of one or more unique biomarkers (e.g., nucleic acid and/or amino acid sequences). In one embodiment the presence of ELS is determined by detecting or determining the presence of an mRNA expression signature indicating the existence of ELS in a sample obtained from the subject. In one embodiment the presence of ELS is determined by detecting or determining the presence of one or more proteins or fragments thereof, indicating the existence of ELS in a sample obtained from the subject

In exemplary embodiments, the ELS-related parameter is a gene signature. In some embodiments, the gene signature comprises one or more, two or more, three or more, four or more, five or more, six or more, seven or more, eight or more, nine or more, ten or more eleven or more, twelve or more, thirteen or more, or all the following genes: CCL21, CCL19, CXCL13, CXCL11, CCL8, CXCL10, CXCL9, CCL2, CCL3, CCL18, CCL5, CLL4 CCL20 and CCL17.

One skilled in the art will appreciate that the gene signature provided herein is merely a non-limiting example and other genes, or combination of genes that are specifically or preferentially expressed in ELS as compared to non-ELS can be used under the methods of the present invention.

In some embodiments, the ELS-related parameter is the binding and imaging of an ELS-binding agent. In some embodiments, said binding and imaging is done in situ or in vitro.

In some embodiments, the determining step comprises the step of obtaining nucleic acid molecules from a biological sample. In some embodiments, the nucleic acids molecules are selected from mRNA molecules, DNA molecules and cDNA molecules. In some embodiments, the cDNA molecules are obtained by reverse transcribing the mRNA molecules. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of a one or more gene such as by amplification of nucleic acids (e.g., PCR, isothermal methods, rolling circle methods, etc.). The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix microarray technology.

The above assays may further provide normalization steps by incorporating the expression of certain normalizing genes, which do not differ significantly in expression levels under the relevant conditions. Typical normalization genes include housekeeping genes.

As used herein, the terms “amplification” or “amplify” mean one or more methods known in the art for copying a target nucleic acid, e.g., the genes listed in Table 1 disclosed herein, thereby increasing the number of copies of a selected nucleic acid sequence. Amplification may be exponential or linear. In a particular embodiment, the target nucleic acid is RNA.

Table 1 listing exemplified ELS-related genes and nucleic acid characterizations

| Gene | mRNA NCBI Reference Sequence | SEQ ID NO: |
|---|---|---|
| CCL21 | NM_002989.3 | 1 |
| CCL19 | NM_006274.2 | 2 |
| CXCL13 | NM_006419.2 | 3 |
| CXCL11 | NM_005409.4 | 4 |
| CCL8 | NM_005623.2 | 5 |
| CXCL10 | NM_001565.3 | 6 |
| CXCL9 | NM_002416.2 | 7 |
| CCL2 | NM_002982.3 | 8 |
| CCL3 | NM_002983.2 | 9 |
| CCL18 | NM_002988.3 | 10 |
| CCL5 | NM_001278736.1 | 11 |
| CCL20 | NM_001130046.1 | 12 |
| CCL17 | NM_002987.2 | 13 |
| LTβ | NM_002341.1 | 14 |

As used herein, “nucleic acid” refers broadly to segments of a chromosome, segments or portions of DNA, cDNA, and/or RNA. Nucleic acid may be derived or obtained from an originally isolated nucleic acid sample from any source (e.g., isolated from, purified from, amplified from, cloned from, or reverse transcribed from sample DNA or RNA). As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product.

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute.

The terms “differentially expressed gene,” “differential gene expression” and their synonyms, which are used interchangeably, refer to a gene whose expression is activated to a higher or lower level in a subject suffering from a disease, specifically HCC, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

The term “significant difference” in the context of the measured expression levels includes up-regulation and/or down-regulation, or combinations thereof of examined genes. In some embodiments, said significant difference is a statistically significant difference such as in mean expression levels, as recognized by a skilled artisan. For example, without limitation, an increase or a decrease of about at least two fold, or alternatively of about at least three fold, compared to a control value is associated with a specific cancer.

In some embodiments, the presence of an ELS-related parameter is determined under imaging assays. In some embodiments, the methods and kits of the present invention provide an ELS-binding agent coupled to an imaging agent. In some embodiments, fluorescence labeling or staining are applied. In some embodiments, the determining step is performed in-situ. In some embodiments, the determining step is performed in-vitro.

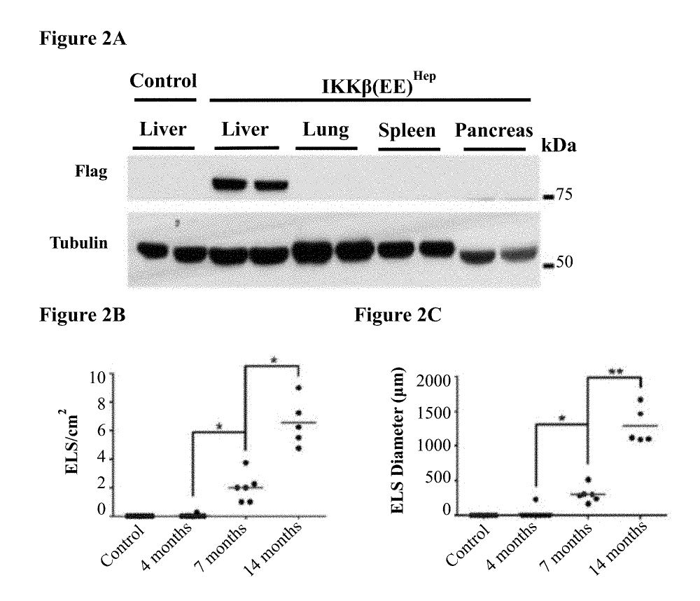

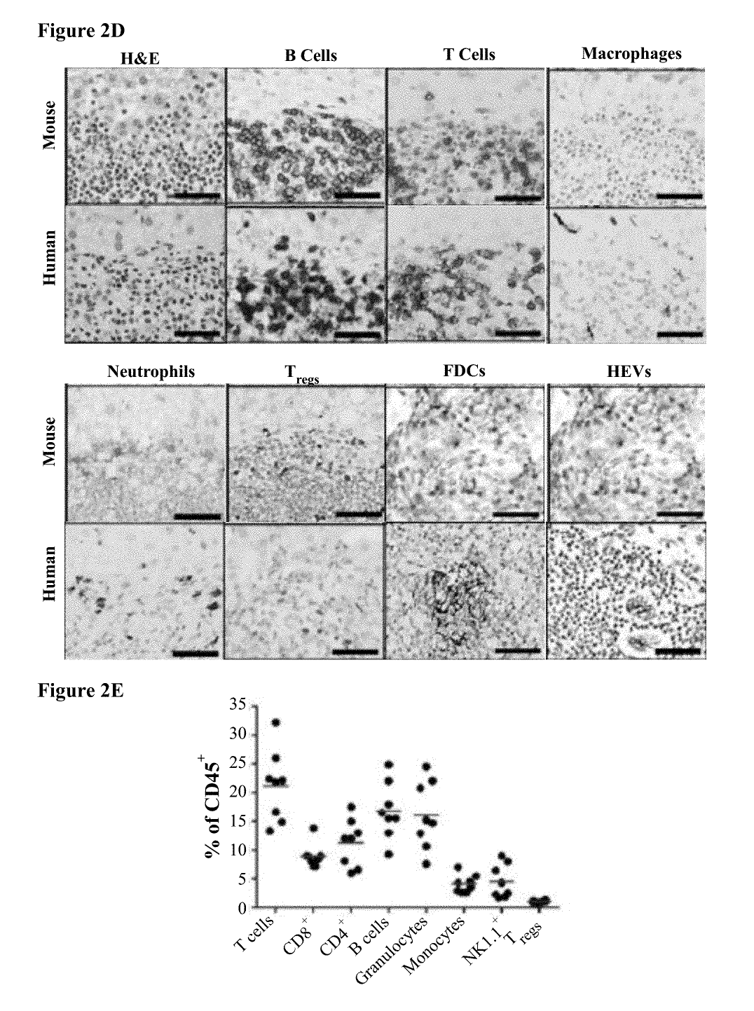

Imaging: One skilled in the art will appreciate that such ELS-binding agent coupled to an imaging agent will be substantially concentrated in ELSs (unlike general chronic inflammation which will show a diffuse signal). In some embodiments, the methods and kits of the present invention provide a dendritic-cell binding agent or a high endothelial venules binding agent. Dendritic-cells and high endothelial venules, as demonstrated herein below, are located in ELS in the liver (see, FIG. 2D, right panels).

In some embodiments, the presence of ELS are determined by detecting or determining the presence of a biomarkers, such as a protein in a sample obtained from the subject. As demonstrated herein below, protein biomarkers are indicative of ELS in the liver (see, FIG. 2D, right panels).

In some embodiments, expression, including level of expression, of protein or polypeptide biomarkers indicative of ELS can be detected through immunohistochemical staining of tissue slices or sections. Additional non-limiting examples of detection of proteins biomarkers include Western blotting, Enzyme-Linked Immunosorbent Assay (ELISA) or Radioimmunoassay (MA) assays employing protein-specific antibodies. Alternatively, protein levels can be determined by constructing an antibody microarray in which binding sites comprise immobilized, preferably monoclonal, antibodies specific to a plurality of proteins. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

The term “sample” or “biological sample” encompasses a variety of sample types obtained from an organism and can be used in a diagnostic, prognostic and/or a monitoring assay. The term particularly relates to solid tissue samples, such as a biopsy specimen or tissue cultures or cells derived therefrom and the progeny thereof. The term “sample” encompasses samples that have been manipulated in any way after their procurement, such as by treatment with reagents, solubilization, or enrichment for certain-components. The term encompasses a clinical sample, and also includes cells in cell culture, cell supernatants, cell lysates and tissue samples. A skilled artisan will appreciate that the sample, according to the methods of the present invention, refers to any liquid or solid material containing nucleic acids, amino acid molecules or other biomarkers indicative of ELSs. The sample may be selected from a biopsy, a tissue sample, a body fluid sample such as blood, plasma, cerbro spinal fluid, urine, saliva, semen or prostatic secretion.

In one embodiment, said sample is a solid tissue sample. In another embodiment, said sample is a tissue biopsy. In another embodiment, the tissue biopsy is a lymph node biopsy. In certain embodiments the tissue is a fresh, frozen, fixed, wax-embedded or formalin-fixed paraffin-embedded (FFPE) tissue.

The “predefined control” is obtained from at least one healthy (e.g., a non-cancerous) individual, a panel of healthy control samples from a set of individuals, and a stored set of data from control individuals.

### Determining Anti-Cancer Therapy

According to some embodiment, there is provided a method of determining subject eligibility for an anti-cancer drug, the method comprising: determining at least one ELS-related parameter in the subject, a parameter higher than a predefined control indicating the subject is eligible for the anti-cancer drug.

In some embodiments, the presence of ELS indicates bad response to anti-cancer drugs. In some embodiments, the anti-cancer drugs are immune checkpoint drugs. Without wishing to be bound by any theory or mechanism of action, immune checkpoint drugs activating the adaptive immune system may augment ELS pro-tumorigenic function.

In accordance with the present invention, there is also provided a method for monitoring the success of anti-cancer drug treatment as defined above, and in particular monitoring the success of anti-ELS therapy, the method comprising: periodically determining the level of at least one ELS-related parameter, a decline in the level indicating success of treatment.

In some embodiments, there is provided a method for determining eligibility for curative liver resection. In some embodiments, determination of ELS score indicates whether liver resection should be performed.

The determination of eligibility for the anti-cancer drugs can be done in order to decide whether the subject should be placed on a specific therapeutic regime, as well as to decide whether the subject may be included in a specific clinical trial.

In some embodiments, the anti-cancer drug is a drug targeting ELS formation. In some embodiments, the anti-cancer drug is a drug targeting ELS pro-tumorigenic function.

Most preferably the drugs are those working via anti-ELS mechanism as will be described below in the connection with the therapeutic aspect of the invention

All the above methods are described in a binary yes/no manner—i.e. if the ELS-related parameters is above a certain threshold the answer is “yes”. However, each of the described methods may be used also to quantify the level of the ELS-related parameter, for example by comparing the level in the tested individual to that of a pre-defined calibration curve, in order to give assurance to the level of likelihood (of having cancer in patient at risk, of recurring cancer in a treated cancer patient) or the level of eligibility to treatment.

### Therapeutic Methods

By another embodiment, the present invention concerns a method for the treatment or for the prevention of cancer in a subject, the method comprising: administering to the subject a therapeutically effective amount of at least one anti-ELS agent.

According to another embodiment, there is provided a method for treating or reducing the likelihood of liver cancer in a subject in need thereof, the method comprising administering to said subject a therapeutically effective amount of anti-ELS agent (e.g., a lymphotoxin inhibitor), thereby treating or reducing the likelihood of liver cancer in said subject.

As demonstrated herein, a mouse model of HCC, displayed abundant ELS which constituted immunopathological microniches. Further, progenitor malignant hepatocytes were shown to appear and thrive in a complex cellular and cytokine milieu until gaining self-sufficiency. Progenitor egression and tumor formation is associated with autocrine production of cytokines previously provided by the niche. ELS develop upon cooperation between the innate and adaptive immune system; facilitated by NF-κB activation and abolished by T cell depletion, therefore these pathways are targets for cancer therapy.

According to another embodiment, there is provided a method for treating or reducing the likelihood of liver cancer in a subject in need thereof, the method comprising the steps of:

a. determining liver-ELS in the subject;

b. administering to said subject a therapeutically effective amount of anti-ELS agent, thereby treating or reducing the likelihood of HCC in said subject.

According to another embodiment, the subject may be treated with agents which reduce the numbers of ELS in the predisposed organ (e.g. by an anti-inflammatory therapy).

The term “treatment or prevention” refers to the treatment of an existing disease, for example for eliminating the cancer, slowing down its progression, slowing or reducing its aggressiveness, metastatic potential or slowing or eliminating its advancement to the next grade cancer. The prevention can be for reducing or abolishing the chances of a subject at risk of developing cancer (as defined above, for example suffering from chronic inflammation of the liver) to eventually develop the disease.

Prevention can also refer to preventing reoccurrence of cancer after anti-cancer therapy for example after resection, more specifically HCC resection.

Non-limiting examples of anti-ELS agents include:

Anti-T and/or anti B cell agents: non-limiting examples of such agents being anti T or anti B antibodies, a non-limiting example being, anti-CD3 antibody or anti CD20 antibody.

Preferably, in order to decrease undesired effect caused by systemic administration of anti-T or anti-B cells agents, the drug is preferentially administered to the ELS, for example by linking the drug and a targeting agent who recognizes ELS. In some embodiments, an agent who recognizes ELS is an integrin expressed in high endothelial venules.

Alternatively, the anti T or anti-B-agents may be administered preferentially to the target organ such as to the liver. This can be done by local placement of the agent (such as present in a sustained release carrier that serves as a depot) at the site of tumor resection (for example HCC resection) so it will exert its effect locally. By another alternative the agent is administered preferentially to the liver by using the portal vain mode of administration. By yet another example, the anti-T or anti B agent may be targeted to the organ, for example liver, by linking it to a molecule that accumulates preferably in that organ. For the liver, such a liver-targeting molecule may be for example bile acids.

By another option, the anti ELS agents are agents that inhibit an agent that is secreted in excess by ELS, and in particular cytokines that are present in ELSs and are involved in the protumorigenic role of ELS. A non-limiting example being at least one of the Lymphotoxin (LT) family members, in particular agents that inhibit LTβ and/or LTα/β and/ or LIGHT, and/or their downstream effectors, CCL17 and CCL20 or are antagonists to the Lymphotoxin β receptor (LTβR).

The inhibition may be at the expression level by various iRNA technologies, or at the protein level by antagonists to the LTPR, neutralizing antibodies or soluble receptors.

A non-limiting example of such as agent being a soluble LTβR fused to a human immunoglobulin Fc portion (LTβR-Ig).

The present invention is based, in part, on the surprising revelation of a liver tumorigenesis program wherein specialized ELS, such as associated with chronic NF-κB activation, foster atypical hepatocytes, that eventually acquire malignant properties.

As demonstrated herein below, ELS—which are frequently present in human livers with HCC promote, rather than counteract, tumor development, as was previously demonstrated for several tumor types. This highlights the existence of contrasting roles of ELS in cancer that may be related to the different cancer types or perhaps reflect alternative phenotypes of ELS.

Shortly following ELS expansion and tumor progenitor egression, distinct tumors carrying similar chromosomal aberrations were observed, indicating that the tumors originated from ELS-nested atypical hepatocytes. The pro-tumorigenic effect of ELS requires a competent adaptive immune system, providing lymphocyte-derived cytokines, that support HCC progenitors until they are ready to egress out of their niches. Related hepatic ELS are commonly found in chronic hepatitis patients who are at risk of developing HCC, hence these ELS provide a microniche function for HCC progenitor expansion and cancer development. Similar to the mouse models presented herein, these human follicles may form due to constitutive IKK activation by chronic hepatitis virus infection (e.g. HBV, HCV).

Thus, the present invention, in some embodiments thereof, demonstrates a critical window of immune-inflammatory action in tumor development, namely intra-niche growth of early tumor progenitors. The immune microniche environment was shown to provide tumor progenitor cells with crucial survival and growth factors. Accordingly, preventing niche assembly or interfering with niche function significantly reduced HCC load in IKKβ(EE)^{Hep} mice.

ELSs are unique micro-anatomic structures which are commonly observed in multiple disease states, including cancer (Pitzalis C, 2014, ibid.). Specifically in the liver, ELS are associated with chronic hepatitis (Scheuer P J, et al. Hepatology 1992, 15(4):567-571; Gerber M A. Clinics in Liver Disease 1997, 1(3):529-541; Iwasaki A, Medzhitov R. Science 2010, 327(5963):291-295).

The IKKβ(EE)^{Hep} model provides, for the first time, functional information for ELS, showing that they provide a unique microenvironment supporting growth of tumor progenitor cells. This notion is corroborated by human studies presented herein, showing a higher probability of late recurrence and death after HCC resection in patients with high hepatic ELS numbers. Of note, late recurrence, occurring 2 years after surgery, is considered to represent de novo carcinogenesis (Sasaki Y, et al. Immunity 2006, 24(6):729-739).

Rag1^{-/-} experiments herein below confirm previous studies showing that certain adaptive immune cells can play an anti-tumor role in hepatocarcinogenesis (i.e. in the absence of IKK-induced ELS formation). Nevertheless, the results exemplified herein, reveal the dramatic pro-tumorigenic potential of adaptive immune cells in forms such as the ELS. In some embodiments, an obvious difference between the pro- and anti-tumorigenic states is formation of highly structured microanatomic structures composed of hundreds of immune cells where the dominant effect is pro-tumorigenic. It is becoming clear that in lymphatic organs, three dimensional structures are key for shaping intercellular communication and cooperation among multiple immune cell types, frequently involving physical cell-cell interactions that are meticulously orchestrated to generate multiple effector mechanisms and bestow different phenotypes on the interacting cells (Verna L, et al, Pharmacology and Therapeutics 1996, 71(1-2):57-81). Thus, the unique structure of the ELS, as well its structural and composition changes over time, explain, and are indicative of, how the adaptive immune system turns from anti- to pro-tumorigenic. In one embodiment, the compact structure of ELS, grouping together high numbers of immune cells, generates a niche containing high concentrations of immune derived cytokines and growth factors providing a tumor-promoting environment.

Formation of ELSs is thought to depend on innate lymphoid cells (LTi) that are recruited to extranodal sites by chemoattracting cytokines (Pitzalis C, 2014, ibid.). The association between NF-κB activation in human livers and the 100% prevalence of ELSs in IKKβ(EE)^{Hep} livers suggests that activation of hepatocyte NF-κB (known to occur in chronic hepatitis of diverse etiologies) plays a role in ELS formation in the liver, linking instigation of epithelial innate immunity and focal activation of adaptive immunity (Yau T O, et al, Journal of Pathology 2009; 217(3):353-361). Although NF-κB is activated throughout the liver of IKKβ(EE)^{Hep} mice, ELSs are focal, suggesting that additional cues are needed. Finding that ELS formation is significantly accelerated by DEN suggests that genotoxic stress could play a role in ELS formation. Supporting this notion is the different distribution of ELSs. In untreated IKKβ(EE)^{Hep} mice, ELSs are evenly distributed between liver zones, whereas in DEN treated mice, the ELSs are largely located in the pericentral zone where DEN is converted from a pro-carcinogen to a carcinogen that can attack DNA. This joint activation of the NF-κB and DNA damage response pathways possibly generates a threshold level of cytokines that are sufficient to invoke the formation of ELSs. Of note, although a previous report suggested that a persistently active IKK transgene expressed in hepatocytes did not lead to HCC induction (Bruix J, Sherman M. Hepatology 2011, 53(3):1020-1022), it is plausible that either the expression level was not sufficiently high or that the follow up time was not long enough. Indeed, mice harboring a single allele of IKKβ(EE) show diminished tumor load compared with mice harboring two alleles, demonstrating that the level of expression of the transgene is important. Furthermore, as demonstrated herein below, HCC developed in two different IKK transgenic strains kept in different facilities.

Egression of tumor cells out of the niche where they first grew is a remarkable phenomenon exemplified herein below in IKKβ(EE)^{hep} mice. Seeds of cancer may germinate in an appropriate microenvironment, yet are capable of leaving the nursing niche and form full-blown malignant tumors only upon acquiring new capabilities. Without wishing to be bound by any theory or mechanism of action, acquisition of niche-independence is a hallmark prerequisite of solid tumors initiated within a supportive niche. One specific mechanism detected herein in IKKβ(EE)^{hep} mice is the acquisition of autocrine LT expression, but others are also very likely to take place. Recently, tumor progenitors were shown to respond to IL-6 from resident tissue macrophages, but later acquire autocrine IL-6 signaling that promotes malignant progression (Schneider C, et al, Gut 2012, 61(12):1733-1743).

### Pharmaceutical Compositions

As used herein, the phrases “agent” and “pharmaceutical composition,” and the like, refer to the combination of one or more active agents, for example, anti-ELS agent selected from anti-CD90, LTβR-IG and CCR6 blockade, and optionally one or more excipients, that is administered to a patient in need of treatment, and can be in any desired form, including for example, in the form of a solution, an aqueous solution, an emulsion, and a suspension.

Pharmaceutical compositions can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005). Suitable formulations can include, but are not limited to, injectable formulations including for example, solutions, emulsions, and suspensions. The compositions contemplated herein may take the form of solutions, suspensions, emulsions, combinations thereof, or any other pharmaceutical dosage form as would commonly be known in the art.

As used herein, the terms “administering”, “administration” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal. The dosage administered will be dependent upon the age, health, and weight of the recipient, kind of concurrent treatment, if any, frequency of treatment, and the nature of the effect desired.

As used herein, the term “carrier,” “excipient,” or “adjuvant” refers to any component of a pharmaceutical composition that is not the active agent. As used herein, the term “pharmaceutically acceptable carrier” refers to a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth;

malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present.

Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington' s Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The carrier may comprise, in total, from about 0.1% to about 99.99999% by weight of the pharmaceutical compositions presented herein.

The term “purified” does not require the material to be present in a form exhibiting absolute purity, exclusive of the presence of other compounds. Rather, it is a relative definition. A peptide is in the “purified” state after purification of the starting material or of the natural material by at least one order of magnitude, 2 or 3, or 4 or 5 orders of magnitude.

The term “substantially free of naturally-associated host cell components” describes a peptide or other material which is separated from the native contaminants which accompany it in its natural host cell state. Thus, a peptide which is chemically synthesized or synthesized in a cellular system different from the host cell from which it naturally originates will be free from its naturally-associated host cell components.

As used herein, the term “substantially pure” describes a peptide or other material which has been separated from its native contaminants. Typically, a monomeric peptide is substantially pure when at least about 60 to 75% of a sample exhibits a single peptide backbone. Minor variants or chemical modifications typically share the same peptide sequence. A substantially pure peptide can comprise over about 85 to 90% of a peptide sample, and can be over 95% pure, over 97% pure, or over about 99% pure. Purity can be measured on a polyacrylamide gel, with homogeneity determined by staining. Alternatively, for certain purposes high resolution may be necessary and HPLC or a similar means for purification can be used. For most purposes, a simple chromatography column or polyacrylamide gel can be used to determine purity.

The definitions of certain terms as used in this specification are provided herein. Unless defined otherwise, all technical and scientific terms used herein generally have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

As used in this specification and the appended claims, the singular forms “a”, “an” and “the” include plural referents unless the content clearly dictates otherwise. For example, reference to “a nucleic acid” includes a combination of two or more nucleic acids, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Materials and Methods

Prognostic evaluation of histological ELS, ELS gene signature, and NF-κB activation in liver tissues from curatively treated HCC patients. Prognostic association of the ELS gene signature was evaluated in genome-wide transcriptome profiles of cirrhotic liver tissues from 82 surgically-treated HCC patients followed for up to 15.6 years (median 7.8 years), previously reported (Hoshida Y, Villanueva A, Llovet JMExpert review of gastroenterology & hepatology 2009, 3(2): 101-103) (NCBI Gene Expression Omnibus accession number GSE10140). For each patient in the cohort, induction of the ELS gene signature was determined by Kolmogorov-Smirnov statistic-based gene set enrichment assessment (Subramanian A, et al. Proceedings of the National Academy of Sciences of the United States of America 2005, 102(43): 15545-15550) implemented in a custom analysis code written in R statistical language (www.r-project.org). Significance of the signature gene induction was determined as a prediction confidence p-value (significance threshold p<0.05) based on null distribution of the statistic generated by random permutation of the samples (n=1,000). Prognostic association of the ELS gene signature was assessed by Kaplan-Meier curves, log-rank test, and multivariable Cox regression modeling adjusted for the 186-gene prognostic/HCC risk signature previously reported and clinical prognostic staging (American Association for Study of Liver Diseases [AASLD] staging system) (Bruix J, et al. Hepatology 2011, 53(3): 1020-1022). No clinical variables were associated with these clinical outcomes.

Correlation between presence of ELS and NF-κB activation was determined by a modulation of 3 experimentally defined NF-κB target gene signatures in HeLa cells (Delhase M, et al, Science 1999, 284(5412):309-313) and primary human fibroblasts and keratinocytes (Saito M, et al, Nature Biotechnology 2001, 19(8): 746-750) and was evaluated in the same transcriptome dataset of 82 HCC patients. Histological ELS features were determined in 66 out of the 82 patients with H&E-stained slides as previously described: vague follicular aggregation (Agg), definite round-shaped cluster of small lymphocytes without germinal center (Fol), and follicles with definite germinal centers composed of large lymphocytes with clear cytoplasm (GC) (M urakami J, et al, Hepatology 1999, 30(1):143- 150). Each section was observed independently by two reviewers that were blinded to the patients' data. A consensus score was reached on a multi-head microscope in cases of discordance. Presence of histological ELS was defined as presence of any of the histological ELS features in ≥50% of portal areas for each patient.

Human liver tissue. Human liver biopsy specimens were obtained from the archives of the Institute of Surgical Pathology, University Hospital Zurich (USZ), Switzerland and kept anonymous. The study protocol was approved by the ethical committee of the “GesundheitSEMirektion Kanton Zurich” (Ref. Nr. StV 26-2005 and KEK-ZH-Nr. 2013-0382) and was in accordance with the Helsinki declaration guidelines. Additional De-identified surgically resected human liver tissues were obtained via Mount Sinai Biorepository (IRB approval HS10-00135).

Mice, HCC induction and anti-Thy1.2 and LTβR-Ig treatments. All animal experiments were performed in accordance with the guidelines of the Hebrew University, University of California, San Diego (UCSD) and NIH for the use of animals for research. Previously described ROSA26-LSL-IKKβ(EE) mice were bred with Albumin (Alb)-Cre mice obtained from Jackson Laboratory (Bar Harbor, Me., stock # 003574) to generate IKKβ(EE)^{Hep} mice. Alb-cre mice served as controls for IKKβ(EE)^{Hep} mice. Alb-IKKβ(EE) mice were generated at the transgenic mouse facility at UCSD as follows: HA-tagged IKKβ(EE) cDNA was PCR-amplified and inserted into a plasmid containing 2.3 kb mouse Alb gene enhancer/promoter, rabbit β-globin second intron, rabbit β-globin polyadenylation signal and SV40 early gene polyadenylation signal. The expression cassette was excised, purified, and injected into fertilized C57BL/6 oocytes to generate founder mice, three of which transmitted the IKKβ(EE) transgene. Rag1^{-/-} mice were purchased from Jackson Laboratory (stock #002216), and Mdr2^{-/-} mice have been described previously. All mice were of a pure C57BL/6 genetic background and were bred and maintained in specific pathogen-free conditions. Only male mice were used. Animals were sacrificed by a lethal dose of anesthesia and perfused through the left ventricle with heparinized PBS followed by buffered formalin.

For hepatocarcinogenesis, mice were injected intraperitoneally (i.p) with 10 mg/kg DEN (Sigma) at 15 days of age. Mice were observed for development of tumors at 9 months of age. To inhibit LTf3R-signaling, a murine LTβR: immunoglobulin G1 fusion protein (LTβR-Ig, Biogen Idec), was used. Mice were i.p injected on a weekly basis with 100 μg of LTβR-Ig or MOPC21 (control murine-IgG1) for 10 consecutive weeks, starting either at 3 weeks of age (denoted early: 3-12), 13 weeks (intermediate: 13-22), or 23 weeks (late: 23-32). Mice were sacrificed at 33 weeks of age to evaluate HCC development (FIG. 16A).

Anti Thyl.2 treatment: mice were injected IP every 2 days for 12 weeks with Thy1.2 antibody (BioXCell, catalog#: BE0066) or LTF-2 antibody for the control group (BioXCell, catalog#: BE0090). Mice were sacrificed at the age of 6.5 months.

Myc-TP53^{-/-} liver tumors were kindly provided by Marie-Annick Buendia (Institut Pasteur, France). Briefly, woodchuck hepatitis virus (WHV)/Myc transgenic mice were mated with p5.3^{-/-} mice to generate mice heterozygous for both the p53 mutation and the Myc transgene. The WHV/Myc p53^{+/delta} idata mice spontaneously developed HCC, which frequently acquired a deletion of the remaining p53 allele. HCCs were further genotyped. Myc-TP5.3^{-/-} HCCs were used as reference for a highly proliferative liver cancer in the 16-gene array analysis.

Serum alanine transaminase (ALT) and aspartate aminotransferase (AST) levels were determined using Reflotron Plus analysis system (Roche).

Examination of mouse H&E sections was performed by three expert liver pathologists (A.W, O.P. & E.P).

Immunohistochemistry (IHC), Immunofluorescence and 3D reconstruction. Antibodies used were directed against: A6 (provided by Valentina Factor and Snorri Thorgeirsson, NIH, USA); B220 (BioLegend clone RA3-6B2); CD3 (Serotec clone CD3-12 or ZYTOMED, cat#RBK024); GFP (Invitrogen cat# A-11122); F4/80 (Serotec clone CI:A3-1); Ly-6G (BD Pharmingen, cat#551459); FDC-M1 (BD Pharmingen clone #FDC-M1); Glutamine Synthetase (GS) (Abcam cat#ab16802); GP73/GOLPH2 (Santa Cruz cat#sc-48011); Foxp3 (eBioscience clone FJK-16s); Ki67 (Thermo Scientific Clone SP6); CDC47 (MCMI) (Santa Cruz, cat#sc-56324); RelA (p65) NeoMarkers catalog #RB-1638); CD44 (eBioscience clone IM7); CD44(V6) (eBioscience clone 9A4); Sox9 (Santa Cruz cat#sc-20095); CK19 [hybridoma TROMA-III, deposited to Developmental Studies Hybridoma Bank (DSHB) by Kemler Rolf]; Collagen IV (Cedarlane Laboratories cat# CL50451AP); E-cadherin (BD clone 36/E-cadherin); Cleaved Caspase 3 (Cell Signaling cat# 9661). Antigen retrievals for B220, GFP, Ki67,CDC47, E-cadherin, RelA, Sox9, CK19 and Cleaved Caspase 3 were performed in 25 mM citrate buffer pH 6.0; for GP73, Ly-6G, GS, Collagen IV, Foxp3, CD44, CD44(V6) and CD3 in EDTA buffer pH 8.0 (Invitrogen)—all by heating to 125° C. for 3 min in decloaking chamber (Biocare Medical). Antigen retrieval for A6 and F4/80 was performed by incubation with 1 mg/ml Pronase XXIV (Sigma). Immunofluorescence was performed on FFPE sections. Fluorophore conjugated secondary antibody used were Donkey anti-Mouse Cy5, Donkey anti-Rat Cy3 (Jackson Immunoresearch) and Alexa-Fluor Donkey anti-Goat 488 (life technologies). Hoechst 33342 (Invitrogen) was used as a nuclei marker.

Antibodies used for human sections were: CD3 (Cell Marque cat#103A); CD15 (Biocare Medical, cat#CM029); CD20 (Cell Marque cat#120R); CD23 (Novacastra Labs clone #1B12); CD68 (Invitrogen clone KP1); Foxp3 (eBioscience clone #236A/E7); HSP 70 (Santa Cruz, cat#sc-24) and LTfβ (Biogen clone B27.B2). IHC staining of human sections was performed with BenchMark XT system (Ventana) using Cell Conditioning 1 (CC1, Ventana) for pretreatment, besides Foxp3 which was stained manually using EDTA buffer pH 8.0 (Invitrogen) for retrieval.

For quantification, stained slides were counted either manually by counting the number of positive cells per 10 high-power fields, or quantified with an Ariol SL-50 automated scanning microscope and image-analysis system (Applied Imaging). Briefly, the frequency of positive cells was assessed with the appropriate module of the Ariol SL-50. For each sample, the percentage of positive cells or the intensity of the staining was determined in 10, arbitrary chosen, fields. Three dimensional reconstruction of ELSs in the liver was done using the μCore software (microDimensions GmbH, Munchen, Germany) using a Mirax Midi Slide Scanner (Carl Zeiss microImaging GmbH, Munchen, Germany) as described (Huang L R, et al. Nat Immunol 2013, 14(6): 574-583).

Immunoblot (IB) and electrophoretic mobility shift assay (EMSA). Antibodies used for IB: Actin (Sigma clone AC-40); FLAG (Sigma clone Anti-Flag M2); p100/p52 (Cell Signaling cat #4882); RelB (Milipore cat #06-1105); Tubulin (Sigma clone DM-1A). To prepare whole-cell lysates, tissues were lysed by mechanical grinding in RIPA buffer (50 mM Tris-HCl pH 7.4, 150mM NaCl, mM EDTA, 1% NP-40 and 0.25% Na-deoxycholate) containing 1× mixture of protease inhibitors (Sigma), 10 mM Na₃VO₄, 10 mM Na₄P₂O₇ and 50 mM NaF. Total cell lysates were separated by SDS-PAGE and assessed by D3 analysis, using sequential probing with the indicated primary Ab and an appropriate anti-IgG conjugated to HRP (Jackson). Immunoreactive bands were detected using ECL detection reagent (Pierce).

For EMSA, IRDye 700 labeled oligonucleotide (LI-COR Biosciences) corresponding to NF-κB specific consensus sequence was used. The binding reaction was performed using Odyssey Infrared electro-mobility shift assay kit (LI-COR Biosciences) according to the manufacturer protocol. Briefly, nuclear proteins were isolated from freshly isolated liver tissue using Cayman Nuclear Extraction Kit (Cayman Chemical Company) and total protein concentration was determined with the BCA Protein Assay Kit (Thermo Scientific). 20 ng of total nuclear protein was mixed with the labeled NF-KB oligonucleotide and left to bind for 30 minutes in the dark. Protein-DNA complexes were resolved by electrophoresis on 4% polyacrylamide Tris/Borate/EDTA (TBE) gel in the dark. Quantitative data was obtained using computerized densitometer and TINA software [version 2.07 d (Raytest)].

RNA in-situ hybridization. RNA in-situ hybridization was performed using the RNAscope 2.0 kit (Advanced Cell Diagnostics) according to manufacturer instructions. Briefly, 4 um FFPE sections were deparaffinized in xylene and pretreated to allow access of probe to target RNA. LTβ specific probe pairs (Advanced Cell Diagnostics) were hybridized to the target RNA at 40° C. in a moist hybridization oven for 2 hours. The signal was amplified using AP-conjugated labeled probes, followed by colorimetric detection using Fast Red as substrate. LTβ mRNA was visualized using standard bright-field microscopy.

Array-based Comparative Genomic Hybridization (aCGH). Agilent oligonucleotide array based CGH for genomic DNA analysis of FFPE samples (Mouse Genome CGH Microarray 4×44 K) was performed on genomic DNA extracted from FFPE liver tissues, according to the protocol provided by Agilent Technologies. Briefly, 500 ng of liver genomic DNA was differentially labeled with Cy3-dCTP (HCC) and Cy5-dCTP (liver tissue from C57BL/6 mice) by random primed labeling (CGH labeling kit for oligo array, Enzo Life Sciences). Liver genomic DNA from C57BL/6 mice was pooled and used as reference DNA. After scanning the array slides, spot fluorescence intensities were extracted using the

Feature Extraction Software (Agilent Technologies), and the raw data text files were used for further analysis. The data were imported into the R statistical platform (http//:www.R-project.org/) and data quality outliers were filtered out using the quality flags as implemented in the Feature Extraction software, such as statistical population outliers or spots with foreground to background ratios smaller than 3. The log₂ ratios of each sample were collated into one matrix and preprocessed and analyzed as follows, using functions from the Bioconductor R package CGHcall (van de Wiel M A, et al. Cancer informatics 2007, 3: 55-63). Missing values were replaced using the values from neighboring probes by an imputation algorithm whereas probes with missing values in more than 30% of samples were excluded from the dataset. The remaining data were median normalized followed by breakpoint detection using a segmentation algorithm (Venkatraman E S, Olshen A B. Bioinformatics 2007, 23(6): 657-663), and the copy number status (loss, normal and gain) of each segment was determined using the CGHcall function (van de Wiel M A, et al. Bioinformatics 2007, 23(7): 892-894). The copy number calls of the single probes were transformed into copy number regions using the CGH regions package (van de Wiel M A, et al. Cancer informatics 2007, 3: 55-63) and plotted for each chromosome according to their physical position.

Proliferation/differentiation analysis of FIG. 3E. The experimental group and tumor histology is indicated by the color code above each column. Clusters were determined by an unsupervised algorithm and designated A, B, the latter further subdivided into B1 and B2. Note that DEN induced HCCs from WT mice are more similar to WT liver parenchyma than IKKβ(EE)^{Hep}, most of which fall into cluster B together with the aggressive Myc-TP5.3^{-/-} mice. Statistical analyses of tumor types in the different clusters: DEN WD-HCCs vs. all IKK HCCs (cluster A vs. B) p<0.0001; DEN classic vs. IKK DEN tumors (both WD and HCC-CCC tumors) (A vs. B) p=0.001; DEN WD-HCCs vs. IKK spontaneous (spon) HCCs (both WD-HCCs and HCC-CCC tumors) (A vs. B) p=0.04; DEN WD-HCCs-IKK HCC-CCC (A vs. B) p=0.006; IKK WD-HCCs vs. IKK HCC-CCC (B1 vs. B2) p=0.007. All p values were determined by two tailed chi-square test. Key: WT liver (purple)=parenchymal liver tissue from untreated 6 months old Alb-cre mice; DEN WD-HCC (green)=well differentiated HCCs from 9 months-old DEN-treated-Alb-cre mice; IKK spon. WD-HCC (blue)=well differentiated HCCs from 20 months-old IKKβ(EE)^{Hep} mice; IKK spon. HCC-CCC (light brown)=undifferentiated mixed HCC-CCCs from 20 months-old IKKβ(EE)^{Hep} mice; IKK DEN WD-HCC (yellow)=well differentiated HCCs from 9 months-old DEN-treated- IKKβ(EE)^{Hep} mice; IKK DEN HCC-CCC (red)=undifferentiated mixed HCC-CCC from 9 months-old DEN-treated- IKKβ(EE)^{Hep} mice; Myc-TP53^{-/-} (brown): HCCs from the very aggressive mouse model for HCC, Myc-TP53^{-/-} mice.

FACS and cell sorter. ELS were dissected under binocular from IKKβ(EE)^{Hep} livers and digested for 30 minutes in 500 μl digestion buffer (HBSS with 0.2 mg/ml collagenase IV and 0.1 mg/ml DNasel) at 37° C. with gentle agitation. The cells were strained through 40 μm filter by washing with cold DMEM, centrifuged for 15 minutes, RBCs were lysed for 10 minutes at 25° with erythrocytes lysis buffer, washed again and resuspended in 0.5 ml DMEM and kept on ice for a few hours until staining. Viability of isolated immune cells was around 85% as determined by Trypan blue. Cells were resuspended and stained in PBS supplemented with 1% fetal calf serum and 1 mM EDTA. Samples were stained and then analyzed by flow cytometry using a Gallios and Kaluza software (Beckman Coulter), or by fluorescence-activated cell sorter (FACS). Antibodies used for flow cytometry and FACS analysis: CD4 (clone RM-4.5, catalog#: 100536, BioLegend), CD8 (clone 53-6.7, catalog#: 65-0081 and 75-008, Tonbo), F4/80 (clone BM8, catalog#: 123127, BioLegend), CD1 lb (clone M1/70, catalog#: 101224, BioLegend), MHCII (clone KH74, catalog#: 115303, BioLegend), CD45.2 (clone 104, catalog#: 109807, BioLegend), NK1.1 (clone PK136, catalog#: 12-5941-83, eBioscience), TCRf3 (clone: H57-597, catalog#: 35-5961, Tonbo), CD44 (clone IM7, catalog#: 103127, BioLegend), CD62L (clone MEL-14, catalog#: 104417, BioLegend), B220 (Catalog#: 553090, BD Pharmingen).

Quantitative PCR analysis. Total RNA was extracted with Trizol (Invitrogen) and was reverse-transcribed by the highcapacity cDNA reverse transcription kit (Applied Biosystems). qPCR reactions were run in triplicate in 384-well plates, and were carried out with SYBR green (Invitrogen) in 7900HT Fast Real-Time PCR System (Applied BioSystems). Results were analyzed using either the Dataassist 2.0 or qBase v1.3.5 softwares. HPRT and PPIA were used as reference genes in both human and murine analyses. Primer sequences are available in Tables 2, 3, and 4 and expression fold and p-values in Tables 5, 6 and 7.

TABLE 2
Primers used for real time PCR proliferation-differentiation 16-(murine)
gene analysis

| Target | Forward primer (SEQ ID NO:) | Reverse primer (SEQ ID NO:) |
|---|---|---|
| NLE1 | GCTGAAGGTGTGGGATGTGA (15) | GAGTCATCTTCTCCATATCCGGA (16) |
| E2F5 | ACCATGGCTGCTCAAAACCT (17) | GCCGTAAAAGAGGAAACACATCAG (18) |
| DLG7 | ACACCTCTGTCTGCCAGCAA (19) | GGCACCTGCTTTCAAGACCA (20) |
| BUB1 | GATTGATTACTTTGGAGTTGCTGC (21) | CATGATGTGAAAAAATTCCTCCC (22) |
| IGSF1 | GGAAGGAGAAAGGCTGGTCAA (23) | CCAAATCCTGGAGCCATCC (24) |
| AFP | GCCTGAACTGACAGAGGAGCA (25) | TTTAAACGCCCAAAGCATCAC (26) |
| DUSP9 | CAATGTCACCCCCAACCTTC (27) | ACAGTTCTGCGACAAGGCCT (28) |
| RPL10A | GGCCTAAACAAGGCTGGCA (29) | CATCGGTCATCTTCACGTGG (30) |
| HPD | GCCCACACTCTTCCTGGAAG (31) | CATTCCAGACCTCACACCATTG (32) |
| GHR | GCAGATGTTCTGAAGGGATGG (33) | TCACCCGCACTTCATGTTCTT (34) |
| ALDH2 | AGGGAGCTGGGCGAGTATG (35) | TGTGTGGCGGTTTTTCTCAGT (36) |
| APOC4 | AGCCACTGGTGACCAGAACC (37) | AGGAGGTGGTCTCTGGAGCTC (38) |
| AQP9 | CCCAGGCTCTTCACTGCTCT (39) | GGTTCGAGTGATGCATTTGGA (40) |
| CYP2E1 | TTTCTGCAGGAAAGCGCG (41) | CTGCCAAAGCCAATTGTAACAG (42) |
| C1S | ATGGGAGATGGGTAAATGACCA (43) | TTAAAGAAGACTTGCCAGGGAAA (44) |
| APCS | TGTTTGTCTTCACCAGCCTTCTT (45) | CGGAAACACAGTGTAAAATTCTGC (46) |

TABLE 3
Primers used for real time PCR analysis (murine)

| Target | Forward primer (SEQ ID NO:) | Reverse primer (SEQ ID NO:) |
|---|---|---|
| Ccl2 | TTAAAAAACCTGGATCGGAACCAA (47) | GCATTAGCTTCAGATTTACGGGT (48) |
| Ccl7 | GCTGCTTTCAGCATCCAAGTG (49) | CCAGGGACACCGACTACTG (50) |
| Cxcl10 | AAGTGCTGCCGTCATTTTCT (51) | CCTATGGCCCTCATTCTCAC (52) |
| Icam1 | TGCGTTTTGGAGCTAGCGGACCA (53) | CGAGGACCATACAGCACGTGCCAG (54) |
| Lta | TCCACTCCCTCAGAAGCACT (55) | AGAGAAGCCATGTCGGAGAA (56) |
| Ltβ | TACACCAGATCCAGGGGTTC (57) | ACTCATCCAAGCGCCTATGA (58) |
| Ccl21 | ATGATGACTCTGAGCCTCC (59) | GAGCCCTTTCCTTTCTTTCC (60) |
| Tnf | CATCTTCTCAAAATTCGAGTGACAA (61) | TGGGAGTAGACAAGGTACAACCC (62) |
| Vcam1 | TACCAGCTCCCAAAATCCTG (63) | CGGAATCGTCCCTTTTTGTA (64) |
| LTβR | TCAAAGCCCAGCACAATGTC (65) | TTATCGCATAGAAAACCAGACTTGC (66) |
| Tnfsf1α | GCAGTGTCTCAGTTGCAAGACATGTCG | CGTTGGAACTGGTTCTCCTTACAGCCAC |
| | G (67) | (68) |
| Ccl20 | ACTGTTGCCTCTCGTACATACA (69) | ACCCACAATAGCTCTGGAAGG (70) |
| Ccl17 | TACCATGAGGTCACTTCAGATGC (71) | GCACTCTCGGCCTACATTGG (72) |
| Cxcl11 | TGTAATTTACCCGAGTAACGGC (73) | CACCTTTGTCGTTTATGAGCCTT (74) |
| Cxcl13 | TCGTGCCAAATGGTTACAAA (75) | ACAAGGATGTGGGTTGGGTA (76) |
| Ccl19 | GCCTCAGATTATCTGCCAT (77) | AGACACAGGGCTCCTTCTGGT (78) |
| Ifnγ | TCAAGTGGCATAGATGTGGAAGAA (79) | TGGCTCTGCAGGATTTTCATG (80) |
| Mfge8 | ATATGGGTTTCATGGGCTTG (81) | GAGGCTGTAAGCCACCTTGA (82) |
| Ccl5 | TTTGCCTACCTCTCCCTCG (83) | CGACTGCAAGATTGGAGCACT (84) |
| Tnfsf10 | CGGGCAGATCACTACACCC (85) | TGTTACTGGAACAAAGACAGCC (86) |
| Cd40lg | CCTTGCTGAACTGTGAGGAGA (87) | CTTCGCTTACAACGTGTGCT (88) |
| Tnfsf14 | TCCGCGTGCCTGGAAA (89) | AAGCTCCGAAATAGGACCTGG (90) |
| Il6 | AGTTGCCTTCGGACTGA (91) | CAGAATTGCCATTGCACAAC (92) |
| Tnfsf11 | GCAGAAGGAACTGCAACACA (93) | GATGGTGAGGTGTGCAAATG (94) |
| Il1α | TGCCAGGAGGATGTCACCT (95) | GGCGGGTCTGGTTTGATGAT (96) |
| Il1β | CAACCAACAAGTGATATTCTCCATG (97) | GATCCACACTCTCCAGCTGCA (98) |
| A20 | CTGGTGTCGTGAAGTCAGGAAG (99) | CCTCAGGACCAGGTCAGTATCC (100) |
| Mcp1 | CTTCTGGGCCTGCTGTTCA (101) | CCAGCCTACTCATTGGGATCA (102) |
| Gadd45β | GCGGCCAAACTGATGAATGT (103) | CTTCTTCGTCTATGGCCAGGA (104) |
| KC | CACCCGCTCGCTTCTCTGT (105) | GCAACACCTTCAAGCTCTGGAT (106) |
| IκB | CTCACGGAGGACGGAGACTC (107) | CTCTTCGTGGATGATTGCCA (108) |
| NF-κB2 | CAGCGAGGCTTCAGATTTCG (109) | CACCTGGCAAACCTCCATG (110) |
| (p100/p52) | | |
| Bcl3 | CAACAGCCTGAACATGGTGCAACT (111) | ATTGTGACAGTTCTTGAGGCCGCT (112) |
| Hprt | GTTAAGCAGTACAGCCCCAAA (113) | AGGGCATATCCAACAACAAACTT (114) |
| Ppia | CGCGTCTCCTTCGAGCTGTTTG (115) | TGTAAAGTCACCACCCTGGCACAT (116) |
| Gapdh | CCACCCCAGCAAGGAGAC (117) | GAAATTGTGAGGGAGATGCT (118) |

TABLE 4
Primers used for real time PCR analysis (human)

| Target | Forward primer (SEQ ID NO:) | Reverse primer (SEQ ID NO: |
|---|---|---|
| CCL17 | ACCGTTGGTGTTCACCGCCC (119) | GGCCCTTTGTGCCCATGGCT (120) |
| CCL20 | GCTACTCCACCTCTGCGGCG (121) | CAGCTGCCGTGTGAAGCCCA (122) |
| LTα | GAGGACTGGTAACGGAGACG (123) | GGGCTGAGATCTGTTTCTGG (124) |
| LTβ | CCACCCTACACCTCCTCCTT (125) | AGTCTGGGCAGCTGAAGGT (126) |
| CXCL10 | TATTCCTGCAAGCCAATTTTGTC (127) | TCTTGATGGCCTTCGATTCTG (128) |
| TNFSF14 | CTGGCGTCTAGGAGAGATGG (129) | CTGGGTTGACCTCGTGAGAC (130) |
| LTβR | GAGAACCAAGGTCTGGTGGA (131) | GAGCAGAAGAAGGCCAGTG (132) |
| TRAIL | TGCGTGCTGATCGTGATCTTC (133) | GGGGTCCCAATAACTGTCATCTT (134) |
| TNFSF11 | CAACATATCGTTGGATCACAGCA (135) | ACAGACTCACTTTATGGGAACC (136) |
| TNFSF1α | CTGCCTCAGCTGCTCCAAA (137) | CGGTCCACTGTGCAAGAAGAG (138) |
| TNF | GGCGCTCCCCAAGAAGACAGG (139) | CCAGGCACTCACCTCTTCCCT (140) |
| CCL2 | CTTCGGAGTTTGGGTTTGCTT (141) | CATTGTGGCCAAGGAGATCTG (142) |
| ICAM1 | ATGCCCAGACATCTGTGTCC (143) | GGGGTCTCTATGCCCAACAA (144) |
| VCAM1 | GCTGCTCAGATTGGAGACTCA (145) | CGCTCAGAGGGCTGTCTATC (146) |
| IL6 | TCGAGCCCACCGGGAACGAA (147) | GCAACTGGACCGAAGGCGCT (148) |
| IL1α | TGGTAGTAGCAACCAACGGGA (149) | ACTTTGATTGAGGGCGTCATTC (150) |
| IL1β | ATGATGGCTTATTACAGTGGCAA (151) | GTCGGAGATTCGTAGCTGGA (152) |
| GAPDH | CCTGGTCACCAGGGCTGC (153) | CCGTTCTCAGCCTTGACGG (154) |

TABLE 5
Summary of P values for real time PCR of ELS gene signature

| | IKKβ(EE)Hep | |
|---|---|---|
| Gene | 14 months | 20 months |
| Ccl2 | 0.0001 | 0.01 |
| Ccl3 | 3.65E−06 | 0.0003 |
| Ccl4 | 0.00004 | 0.002 |
| Ccl5 | 0.0002 | 0.042 |
| Ccl8 | 0.002 | 0.035 |
| Ccl19 | 0.050 | 0.040 |
| Ccl21b | 0.030 | 0.110 |
| Cxcl9 | 0.002 | 0.0004 |
| Cxcl10 | 0.001 | 0.030 |
| Cxcl11 | 0.030 | 0.440 |
| Cxcl13 | 0.110 | 0.030 |
| n | 12 (control), 7 (14 months) | 12 (control), 11 (20 months) |

TABLE 6
Summary of relative gene expression values shown in FIG. 7A

| | Parenchyma | | | | | HCC | | |
|---|---|---|---|---|---|---|---|---|
| | | 3 | 6 | 9 | | 9 | | |
| | 6 | month | month | month | 20 | month | 20 | |
| Gene | month | DEN | DEN | DEN | month | DEN | month | ELS* |
| Ltβr | 1.23 | 1.41 | 1.05 | 0.86 | 0.89 | 1.03 | 0.41 | 5.62 |
| Tnfrsf1α | 1.26 | 1.68 | 1.04 | 1.26 | 0.82 | 1.20 | 0.56 | 9.70 |
| Cxcl11 | 0.70 | 1.78 | 1.13 | 1.23 | 0.91 | 0.85 | 0.53 | 3.23 |
| Cxcl13 | 2.49 | 2.97 | 0.51 | 1.55 | 5.02 | 1.32 | 6.70 | 1.01 |
| Ccl21 | 1.71 | 1.95 | 2.80 | 2.69 | 1.02 | 0.70 | 8.22 | 3.34 |
| Ccl19 | 1.75 | 2.54 | 3.24 | 2.56 | 1.08 | 0.74 | 7.44 | 28.48 |
| Ifnγ | 2.30 | 2.39 | 7.58 | 0.74 | 2.27 | 0.96 | 3.71 | 18.60 |
| Cxcl10 | 4.71 | 1.12 | 1.18 | 2.86 | 1.94 | 1.58 | 1.94 | 86.93 |
| Il1α | 2.05 | 1.19 | 1.71 | 1.99 | 1.74 | 2.02 | 1.21 | 2.21 |
| Mfge8 | 2.74 | 31.18 | 1.90 | 1.64 | 7.98 | 2.37 | 1.82 | 23.19 |
| Ltα | 2.48 | 1.79 | 2.86 | 3.51 | 1.81 | 1.12 | 8.85 | 1.16 |
| Il1β | 3.34 | 2.51 | 2.91 | 2.74 | 3.20 | 3.19 | 2.00 | 14.75 |
| Ccl5 | 2.55 | 2.30 | 1.74 | 2.02 | 3.96 | 3.98 | 10.30 | 21.26 |
| Vcam1 | 4.10 | 3.11 | 3.06 | 3.59 | 3.97 | 4.13 | 4.16 | 118.24 |
| Tnf | 4.20 | 512.60 | 3.21 | 4.46 | 3.16 | 4.76 | 3.83 | 29.26 |
| Tnfsf10 | 5.43 | 5.03 | 4.34 | 4.45 | 3.96 | 3.61 | 0.78 | 4.20 |
| Cd40lg | 1.54 | 3.79 | 4.76 | 5.09 | 2.08 | 5.34 | 13.37 | 7.05 |
| Tnfsf14 | 3.10 | 2.33 | 4.70 | 4.64 | 1.53 | 3.16 | 4.23 | 14.96 |
| Icam1 | 12.00 | 5.06 | 11.98 | 6.13 | 2.30 | 3.74 | 2.49 | 4.54 |
| Ccl7 | 5.45 | 3.12 | 3.76 | 9.51 | 2.96 | 2.76 | 2.85 | 94.89 |
| Ccl2 | 5.99 | 3.12 | 3.84 | 6.92 | 3.63 | 5.93 | 4.22 | 117.12 |
| Il6 | 13.32 | 2.12 | 3.34 | 12.47 | 9.31 | 12.04 | 1.59 | 13.72 |
| Tnfsf11 | 0.61 | 2.66 | 3.46 | 12.02 | 28.24 | 13.01 | 36.92 | 34.53 |
| Ltβ | 1.44 | 6.02 | 13.20 | 11.98 | 35.48 | 35.46 | 23.61 | 80.12 |
| Cel17 | 2.75 | 18.47 | 16.63 | 98.83 | 50.93 | 176.82 | 235.74 | 76.31 |
| Ccl20 | 2.31 | 81.20 | 135.60 | 230.04 | 177.90 | 1024.13 | 829.19 | 30.03 |

TABLE 7
Summary of P values for real time PCR of cytokines

| | | | | | | HCC* | | | | |
|---|---|---|---|---|---|---|---|---|---|---|
| | 3 | Parenchyma, DEN treated* | | | | 9 | 20 | | | |
| | month | 3 | 6 | 9 | 20 | month | month | | Human | |
| Gene | UT* | month | month | month | month | DEN | UT | ELS* | HCV ** | LTβR-Ig*** |
| Cxcl13 | 0.0926 | 0.2596 | 0.1838 | 0.0615 | 0.0615 | 0.7063 | 0.1370 | 0.1488 | N.T. | 0.0053 |
| Cel17 | 0.0054 | 0.0003 | 0.0014 | 0.0005 | 0.0005 | 0.0002 | 0.0023 | 2.58E−10 | 1E−06 | 0.0082 |
| Ccl2 | 0.0002 | 0.0049 | 0.0178 | 0.0240 | 0.0240 | 0.0075 | 0.0106 | 8.13E−11 | 6E−06 | 0.0915 |
| Ccl20 | 0.6742 | 1.31E−06 | 1.36E−06 | 0.0057 | 0.0057 | 0.0001 | 0.0031 | 3.87E−11 | 0.0019 | 0.0103 |
| Ccl5 | 0.0062 | 0.0808 | 0.4484 | 0.0208 | 0.0208 | 0.0982 | 0.0768 | 0.0013 | N.T. | 0.0002 |
| Ccl7 | 0.0006 | 0.0015 | 0.0108 | 0.0359 | 0.0359 | 0.1035 | 0.1734 | 8.32E−11 | N.T. | 0.0262 |
| Cd40lg | 0.1021 | 0.0024 | 0.0029 | 0.2631 | 0.2631 | 0.0081 | 0.0246 | 0.0001 | N.T. | 0.1260 |
| Cxcl10 | 0.0033 | 0.8258 | 0.5729 | 0.0860 | 0.0860 | 0.4216 | 0.4875 | 0.000003 | 0.0370 | 0.3197 |
| Cxcl11 | 0.5857 | 0.0036 | 0.4292 | 0.4729 | 0.4729 | 0.8116 | 0.4155 | 0.0001 | N.T. | 0.2757 |
| Ccl19 | 0.2301 | 0.0496 | 0.0005 | 0.7781 | 0.7781 | 0.6620 | 0.2193 | 0.00001 | N.T. | 0.0105 |
| Icam1 | 0.000001 | 0.0144 | 0.0001 | 0.0035 | 0.0035 | 0.0076 | 0.0823 | 2.28E−08 | 0.1610 | 0.1730 |
| Il1a | 0.0713 | 0.4147 | 0.1673 | 0.0116 | 0.0116 | 0.0176 | 0.8093 | 0.0021 | 0.5236 | 0.2533 |
| Il1b | 0.0071 | 0.1491 | 0.0440 | 0.0087 | 0.0087 | 0.0598 | 0.4108 | 0.00003 | 0.4928 | 0.1140 |
| Il6 | 0.0033 | 0.2572 | 0.0111 | 0.0012 | 0.0012 | 0.0391 | 0.7447 | 0.00004 | 0.8941 | 0.7764 |
| Ifnγ | 0.3178 | 0.1034 | 0.0211 | 0.2606 | 0.2606 | 0.9604 | 0.2503 | 0.0004 | N.T. | 0.0240 |
| Tnfsf14 | 0.0014 | 0.0961 | 0.0304 | 0.0971 | 0.0971 | 0.0024 | 0.0567 | 0.0001 | 0.0491 | 0.6710 |
| Ltα | 0.0323 | 0.2310 | 0.0037 | 0.2640 | 0.2640 | 0.4882 | 0.0232 | 0.00003 | 0.0208 | 0.0624 |
| Ltβ | 0.5690 | 0.0007 | 0.0007 | 0.0049 | 0.0049 | 0.00004 | 0.0103 | 4.02E−08 | 0.0003 | 0.0242 |
| Ltβr | 0.0813 | 0.6399 | 0.9017 | 0.5380 | 0.5380 | 0.9260 | 0.1797 | 0.1036 | 0.0166 | 0.0642 |
| Mfge8 | 0.0090 | 0.0040 | 0.2798 | 0.0001 | 0.0001 | 0.1908 | 0.4487 | 0.0001 | N.T. | 0.0032 |
| Tnfsf11 | 0.5773 | 0.0102 | 0.1255 | 0.0097 | 0.0097 | 0.0009 | 0.0181 | 0.0001 | 0.7308 | 0.0201 |
| Ccl21 | 0.0760 | 0.1074 | 0.0044 | 0.9019 | 0.9019 | 0.5659 | 0.1225 | 0.00002 | N.T. | 0.0113 |
| Tnf | 0.0011 | 0.0004 | 0.2389 | 0.0294 | 0.0294 | 0.0757 | 0.5095 | 0.0003 | 0.0090 | 0.0019 |
| Tnfsf1α | 0.0812 | 0.4144 | 0.8969 | 0.4248 | 0.4248 | 0.2750 | 0.3068 | 0.0035 | 0.0006 | 0.6167 |
| Tnfsf10 | 0.0005 | 0.00003 | 0.0412 | 0.0044 | 0.0044 | 0.0606 | 0.6662 | 0.0001 | 0.0013 | 0.4724 |
| Vcam1 | 0.0109 | 0.0022 | 0.0097 | 0.0181 | 0.0181 | 0.0234 | 0.1290 | 4.03E−08 | 0.1871 | 0.0159 |
| n | 4, 5 | 6, 6 | 4, 5 | 5, 5 | 5, 4 | 5, 5 | 1, 5 | 4, 7 | 12, 43 | 6, 7 |

*Summary of P values for FIG. 7A.
** Summary of P values for FIG. 7B.
***Summary of P values for FIG. 8A.
n represents number of mice in control, experimental group respectively
UT = untreated, N.T. = not tested.

Digital PCR analysis. Genomic DNA was extracted from fresh frozen tissue with DNeasy (QIAGEN, Catalog# 69504) and from FFPE tissue with QIAamp DNA Micro Kit (QIAGEN, Catalog# 56304) and used for digital PCR analysis with the following TAQMAN probes: Rgs2, Gab2 (Applied

Biosystems, catalog# AB- 4400291), Tert (Applied Biosystems, catalog# AB-4458368). High-throughput droplet digital PCR for quantitation of DNA copy number was done as described (Hindson BJ, et al. Anal Chem 2011, 83(22): 8604-8610).

Statistical analysis. Results are expressed as mean ±SEM. Statistical significance (p<0.05) was determined by either two-tailed Student's t-test, two tailed chi-square test or Fisher's exact test. For correlation analysis in mRNA expression levels, either Spearman or Pearson correlation tests at p<0.05 were used. Data was processed using Microsoft Excel or GraphPad Prism 6.0.

### Example 1

### ELS Depend on NF-κB and Signify Poor Prognosis in Human HCC

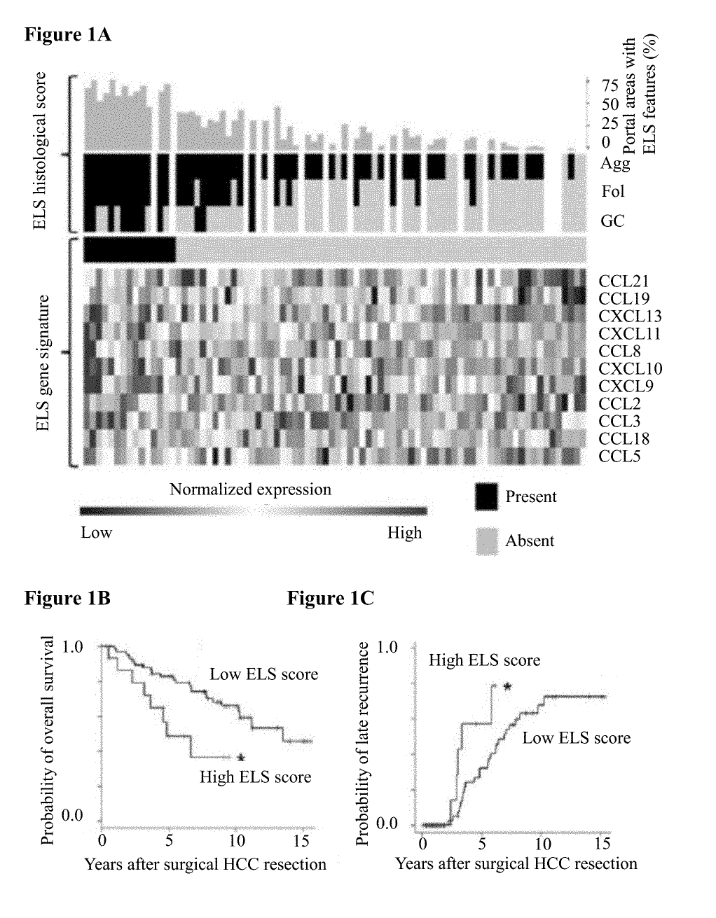

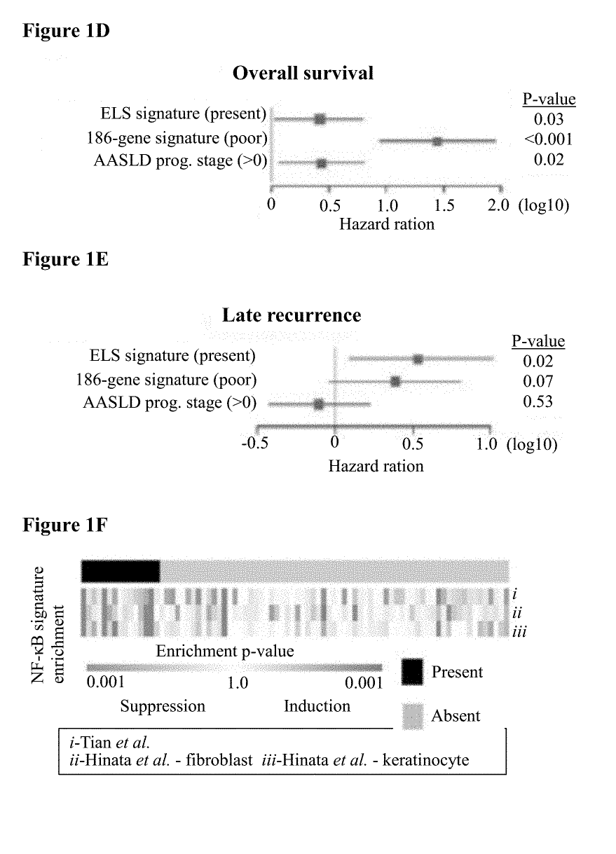

To assess the relationship between hepatic ELS prevalence and prognosis in human HCC, the number of ELS in the non-neoplastic liver parenchyma in a well-characterized cohort of 82 patients having undergone HCC resection were quantified, for which clinical data, histological slides and gene expression data of the liver parenchyma was obtained (Hoshida Y, et al. The New England journal of medicine 2008, 359(19): 1995-2004). ELS prevalence was histologically assessed in 66 cases (the subset of cases with H&E-stained slides), using a published quantification scale (Murakami J, et al. Hepatology 1999, 30(1): 143-150) (FIG. 1A and FIG. 9A). This analysis revealed that in contrast to colon, breast, lung and skin cancers, a high histological ELS score was associated with increased risk for late recurrence and a trend towards decreased overall survival after HCC resection (FIG. 9B-D).

A 12-gene signature was shown to accurately assess the presence of ELSs in some human tissues (Coppola D, et al. Am J Pathol 2011, 179(1): 37-45). Using expression data available for these patients, a strong correlation was found between the histological ELS score and a modified 11 gene ELS signature (FIG. 1A, p<0.001), confirming its utility in the liver and enabling expansion of the analysis to include all 82 patients with transcriptome profiles. Fifteen out of 82 patients (18%) presented the ELS gene signature in the liver parenchyma (FIG. 1A), which was significantly associated with poor survival of HCC patients after curative surgical resection (FIG. 1B, p=0.01) and increased risk of late, but not early recurrence (FIG. 1C and FIG. 9E; p=0.03 and p=0.34, respectively). Of note, multivariable analysis showed that the ELS gene signature is an independent prognostic factor from the 186-gene prognostic-HCC risk gene signature previously identified in this cohort (Hoshida Y, 2008, ibid.), as well as the clinical prognostic staging system (FIG. 1D,E). Late recurrence, occurring 2 years after surgery, is considered to represent de novo carcinogenesis from the inflamed liver, while early recurrence occurring within 2 years of surgery results from dissemination of primary tumor cells (Hoshida Y, et al. Expert review of gastroenterology & hepatology 2009, 3(2): 101-103). Collectively, the clinical cohort analysis suggested that ELSs are associated with de novo HCC development in chronically inflamed and fibrotic or cirrhotic human livers.

The mechanisms underlying ELS formation in general and in cancer in particular are mostly obscure (Pitzalis C, 2014, ibid.; Drayton D L, et al, Nature Immunology 2006, 7(4):344-353). To identify signaling pathways that could initiate or facilitate ELS development in HCC, gene set enrichment analysis (GSEA) was performed, comparing gene expression in the liver parenchyma between patients with high vs. low ELS gene signatures. This analysis highlighted the interferon response and NF-KB signaling as top candidates (Table 8).

TABLE 8
Gene Set Enrichment Analysis (GSEA)

| | Normalized | | False |
|---|---|---|---|
| | enrichment | | Discovery |
| Gene set | score | p-value | Rate |
| INTERFERON GAMMA | 2.83 | <0.001 | <0.001 |
| RESPONSE | | | |
| INTERFERON ALPHA RESPONSE | 2.81 | <0.001 | <0.001 |
| ALLOGRAFT REJECTION | 2.30 | <0.001 | <0.001 |
| TNFA SIGNALING VIA NFKB | 2.21 | <0.001 | <0.001 |
| IL6 JAK STAT3 SIGNALING | 2.07 | <0.001 | <0.001 |
| KRAS SIGNALING UP | 1.96 | <0.001 | 0.001 |
| INFLAMMATORY RESPONSE | 1.92 | <0.001 | 0.001 |
| COMPLEMENT | 1.88 | <0.001 | 0.001 |
| PROTEIN SECRETION | 1.75 | <0.001 | 0.004 |
| APOPTOSIS | 1.69 | <0.001 | 0.009 |
| E2F ARGETS | 1.50 | 0.009 | 0.041 |
| ANDROGEN RESPONSE | 1.46 | 0.013 | 0.047 |

Molecular pathways associated with ELS signature in the liver parenchyma of a cohort of patients with high or low ELS gene signatures undergoing HCC resection (Gene Set Enrichment Analysis).

Further analysis of the correlation between activation of NF-KB signaling and hepatic ELSs using 3 different published NF-KB signatures confirmed the association for NF-κB (FIG. 1F and FIG. 9F).

These findings suggest that activation of the I kappa B kinase (IKK)-NF-κB signaling pathway could be an important mediator of hepatic ELS generation.

### Example 2

### Persistent IKK Activation in Hepatocytes Induces ELSs

In order to examine the contribution of NF-κB signaling to generation of ELS and HCC, an animal model was developed. To activate the IKK-NF-κB signaling pathway in hepatocytes, R26Stop^{FL}Ikk2ca mice (Sasaki Y, et al. Immunity 2006, 24(6): 729-739) were bred with Albumin-cre (Alb-cre) mice (Postic C, Magnuson M A. Genesis 2000, 26(2): 149-150). The resulting IKKβ(EE)^{H}eP mice express constitutively active IKKβ(EE) in hepatocytes (FIG. 2A) and show nuclear NF-KB and transcriptional activity comparable in their amounts to Mdr2^{-/-} (also known as Abcb4^{-/-}) mice, a model of chronic hepatitis (Pikarsky E, et al. Nature 2004, 431(7007): 461-466), but lower than TNF treated mice (FIG. 10A-D), suggesting that IKKβ(EE)^{Hep} mice display a level of innate immune activity which is similar to that present in common forms of chronic hepatitis. The livers of 3 month old IKKβ(EE)^{Hep} mice lacked overt histopathology (FIG. 10E). At 7 months IKKβ(EE)^{Hep} mice revealed mild increases in liver macrophages, liver damage markers and hepatocyte proliferation (FIG. 10F-K). Importantly, multiple ELSs were apparent in livers of 7 month old IKKβ(EE)^{Hep} mice, gradually growing in both size and number (FIG. 2B,C). Immunohistochemical staining revealed that ELSs were composed of T and B lymphocytes, neutrophils (located in the ELS periphery), NK cells, macrophages, T regulatory (T_{reg}) cells, follicular dendritic cells and contained high endothelial venules, confirming that these were bona fide ELSs (FIG. 2D).

Next, ELSs present in the parenchyma of human livers with hepatitis (that were resected for HCC) were analyzed and their immune cell composition were compared to that of hepatic ELSs in IKKβ(EE)^{Hep} mice. Histological analysis revealed that ELSs in IKKβ(EE)^{Hep} mice were highly similar to their human counterparts (FIG. 2D). Flow cytometry of single cell suspensions of mouse ELSs confirmed the immunohistochemical analysis (FIG. 2E and FIG. 10L,M). B and T lymphocyte compartmentalization, another characteristic feature of ELSs, was also observed in ELSs in IKKβ(EE)^{Hep} mice and human hepatitis (FIG. 10N). Furthermore, the ELS gene signature was significantly upregulated in liver parenchymas of IKKβ(EE)^{Hep} mice compared to control Alb-cre mice (FIG. 100 and Table 7). Thus, persistent IKK activation in hepatocytes could be a key mediator of hepatic ELS formation in human hepatitis.

### Example 3

### Hepatic ELS Herald Aggressive HCCs in IKKβ(EE)^{Hep} Mice

At 20 months of age, 100% of IKKβ(EE)^{Hep} mice developed HCC compared to 8% of control Alb-cre mice (FIG. 3A-C, p<0.0001). Histological analysis revealed that approximately half of IKKβ(EE)^{Hep} tumors were well differentiated HCCs (WD-HCC); the remaining tumors were mixed cholangio-hepatocellular carcinomas (HCC-CCC, FIG. 3D), recognized by the presence of malignant glandular structures. Immunostaining for the HCC markers A6 and glutamine synthetase (GS), the proliferation marker Ki-67 and the matrix associated collagen IV (whose expression is downregulated in HCC), and the presence of metastases to lymph nodes and lungs confirmed that these were aggressive malignant HCCs (FIG. 11A-C). Notably, mice harboring a single allele of IKKβ(EE) showed decreased ELS number and size at 14 months (FIG. 11D,E) followed by a similar decrease in HCC load at 20 months, when compared to mice harboring two such alleles (FIG. 11F,G), underscoring an NF-κB dose-dependent ELS phenotype and an association between ELSs and HCC, respectively.

Transgenic mice, in which IKKβ(EE) is driven by the Albumin promoter [Alb-IKKβ(EE) mice], were generated. Of note, these mice also displayed ELSs and mild inflammation, followed with development of HCC (FIG. 11H-J), corroborating the hepatocarcinogenic effect of constitutive IKKβ(EE) expression in hepatocytes in a distinct mouse model. Treating IKKβ(EE)^{Hep} mice with the hepatic carcinogen diethylnitrosamine (DEN) accelerated the appearance of ELSs (now appearing at 3 months), and HCCs (appearing at 9 months), without significantly altering their histological and molecular characteristics (FIG. 11K-R).

Analysis of expression of a 16 gene set for assessing HCC aggressiveness (Cairo S, et al. Cancer cell 2008, 14(6): 471-484), revealed that HCCs from DEN-treated wild-type mice tended to cluster with wild-type livers, while HCCs from IKKβ(EE)^{Hep} mice clustered with aggressive HCCs from transgenic mice overexpressing Myc in hepatocytes together with germline deletion of TP53 (Myc-TP53^{-/-}, FIG. 3E, 7 out of 10 compared with 2 out of 20, p=0.002, Fisher's exact test). Tumors displaying the HCC-CCC morphology, which were only detected in the IKKβ(EE)^{Hep} mice and not found in control DEN-treated ones, clustered with the more aggressive group (FIG. 3E). Of note, a difference in the gene expression pattern between spontaneous and DEN induced HCCs in IKKβ(EE)^{Hep} mice was not detect. Array comparative genomic hybridization (CGH) analyses of HCCs from DEN-treated Alb-cre control (n=12), DEN-treated IKKβ(EE)^{Hep} mice (n=11) and 20-months old IKKβ(EE)^{Hep} mice (n=13) revealed chromosomal aberrations in all HCC samples, confirming their neoplastic nature. Data are stored and available from ArrayExpress (https://www.ebi.ac.uk/arrayexpress/) accession number E-MTAB-3848. Mixed HCC-CCC tumors were more complex than WD-HCC (FIG. 11S). To validate the array CGH analysis, digital PCR probes directed at two genes, Rgs2 and Gab2, targeting the two most common genomic amplifications, were prepared. This analysis revealed a 90% concordance with results of the array CGH analysis. In addition, analysis of 13 additional IKKβ(EE)^{Hep} HCCs revealed the presence of amplification of Rgs2 gene copy number in 38% and amplification of Gab2 gene copy number in 30% of the HCCs tested (FIG. 11T). Taken together, these data confirmed that IKKβ(EE)^{Hep} mice develop aggressive HCCs with a 100% penetrance.

### Example 4

### HCC Progenitors First Appear Inside ELS and Later Egress

Careful histological analysis revealed that the earliest malignant hepatocytes (noted at 3 and 7 months in DEN-treated and untreated IKKβ(EE)^{Hep} mice, respectively), appeared first within newly-formed ELSs. These malignant hepatocytes were double positive for GFP (expressed from the hepatocyte specific IKKβ(EE) transgene) and E-cadherin, confirming their hepatocyte origin and epithelial phenotype (FIG. 4A), and expressed multiple markers of HCC progenitors including A6, GP73 (GOLPH2), Sox9, CD44v6²³ and CK19 (FIG. 4B). At these earliest time points, HCC progenitors were largely found in ELSs and not elsewhere in the liver parenchyma (FIG. 12A-C). GFP expression proved that these cells were derived from hepatocytes expressing the Alb-cre transgene (FIG. 12D). These small clusters gradually grew, initially within ELSs and later on, migrating out to form visible tumors (FIG. 4C and FIG. 12E). This histological sequence, of small groups of cells first appearing within ELSs, followed with gradual coalescence of groups of cells within the follicle boundary, which finally grew out into full-blown HCCs, was consistently seen in all IKKβ(EE)^{Hep} mice of appropriate age in both spontaneous and DEN-treated groups (FIG. 12F,G). To unequivocally prove the neoplastic nature of the epithelial cells that grew within the ELSs, laser capture micro-dissection was used to collect enriched populations of these cells. Indeed, 3 out of 11 lesions harbored amplification of Rgs2 and 1 out of 11 harbored amplification of Gab2 (FIG. 12H), corroborating the neoplastic nature of these lesions and providing a genetic link between the malignant cells that were thriving within the ELSs and HCCs of IKKβ(EE)^{Hep} mice.

Of note, several months after the first appearance of ELSs, clusters of malignant hepatocytes budding from the ELSs were consistently observed (FIG. 121). These clusters were either in continuity with or slightly separated from the intra-ELS malignant cells. To better visualize the egression of clusters of malignant cells, the inventors co-immunostained serial sections with antibodies against CD44v6 (a marker for HCC progenitor cells²³) and B220 and generated 3 dimensional reconstructions of ELSs. The resulting 3 dimensional representations clearly showed clusters of malignant cells budding out of ELSs (FIG. 4D).

Although the IKKβ(EE) transgene is expressed throughout the parenchyma, ELSs are focal, suggesting that additional factors trigger ELS formation. Finding that ELS appearance was accelerated by DEN administration (FIG. 11L,M) suggests that tissue damage could be of relevance, either by focal enhancement of hepatocyte NF-κB activation, or by triggering another cooperating pro-inflammatory pathway. To test this possibility, the microanatomical localization of ELSs was assessed with respect to different liver zones. DEN is converted to its active metabolite in pericentral hepatocytes. Thus, if genotoxic stress is directly involved in formation of ELSs, the latter will be localized to pericentral regions. Immunostaining with GS (a marker of pericentral hepatocytes) revealed that ELSs were evenly distributed in the 3 liver zones in untreated IKKβ(EE)^{Hep} mice; however, in DEN treated IKKβ(EE)^{Hep} mice ELSs were almost entirely limited to the pericentral zone (FIG. 4E and FIG. 12J, p<0.0001), indicating a causal relationship between genotoxic stress and ELS formation.

To find out if HCC progenitors are also found in ELSs of human patients, liver parenchyma from human livers resected for HCC was analyzed. Triple immunofluorescence with antibodies against the human HCC markers HSP70 and SOX9 together with CK19 (which marks reactive ductular cells), revealed the presence of cells with HCC progenitor features within the human ELSs (FIG. 4F), attesting to a common ELS-related pathogenic mechanism in human HCC and the mouse model.

### Example 5

### Depletion of ELS Markedly Attenuates Murine HCC

The adaptive immune system is commonly considered a defense mechanism against cancer progression; accordingly human HCCs showing marked lymphocytic infiltration were found to have a better prognosis (Wada Y, et al. Hepatology 1998, 27(2): 407-414) and Rag1^{-/-} mice, lacking an adaptive immune system, were shown to have more HCCs after DEN treatment (Schneider C, et al. Gut 2012, 61(12): 1733-1743). Yet, despite its defense function, immune activation can result in various pathologies including cancer (Okin D, Medzhitov R. Curr Biol 2012, 22(17): R733-740; Karin M, et al. Cell 2006, 124(4): 823-835).

To test the functional role of ELSs in hepatocarcinogenesis, IKKβ(EE)^{Hep} mice were bred with Rag1^{-/-} mice which completely lack B and T cells. Consistent with a previous report (Schneider C, ibid.), there was a small increase in HCC numbers in DEN treated Rag1^{-/-} mice (FIG. 5A-D). As expected, Rag1 deletion in IKKβ(EE)^{Hep} mice resulted in complete elimination of ELSs. However, in stark contrast with its pro-tumorigenic effect in Rag1^{-/-} mice, loss of the adaptive immune system in IKKβ(EE)^{Hep} -Rag1^{-/-} mice dramatically attenuated hepatocarcinogenesis; most, if not all, of the pro-tumorigenic effect of the IKKβ(EE) transgene was lost in the absence of Ragl (FIG. 5A-D). Tumors that do develop in Rag1^{-/-} IKKβ(EE)^{Hep} mice were exclusively typical, WD-HCC and were negative for most HCC progenitors markers (FIG. 5E-G). Comparing proliferation, apoptosis and RelA (p65) nuclear accumulation between the HCCs in IKKβ(EE)^{Hep}-Rag1^{-/-1} to well differentiated HCCs of IKKβ(EE)^{Hep} mice did not reveal significant differences (FIG. 13A-F), arguing against a cell-autonomous effect of Rag1 deficiency on HCC growth.

Taken together these data suggest that generation of a focal immune microniche is dependent on a functional adaptive immune system and that the immune microniche promotes HCC.

To test whether ablation of ELS function after induction of tumors by DEN could still affect HCC formation in IKKβ(EE)^{Hep} mice, anti-Thy1.2 antibody was administered, to ablate T cells and potentially also innate lymphoid cells and NK cells which were also reported to respond to anti-Thy1.2 treatment, to mice between 18 to 30 weeks of age. Control mice received an isotype matched control antibody (FIG. 14A). Immunostaining livers for CD3 confirmed almost complete T cell ablation (FIG. 6A). Indeed, anti-Thy1.2 treatment restricted ELS development (FIG. 6B), and markedly reduced HCC multiplicity and burden in IKβ(EE)^{Hep} mice (FIG. 6C-E and FIG. 14B-D). Thus, the adaptive immune system plays a strong pro-tumorigenic effect in IKβ(EE)^{Hep} mice, which takes place after acquisition of the initiating tumor mutations.

### Example 6

### ELSs Express High Amounts of Growth Promoting Cytokines

It was hypothesized that cytokines secreted by adaptive immune cells possibly present at high concentrations within ELSs, could underlie their tumor-promoting effects. To identify pro-tumorigenic signals operative within ELSs, expression of multiple cytokines in liver parenchyma, ELSs and HCCs from IKkβ(EE)^{Hep} mice and in human chronic viral hepatitis, were measured. Among others, Lymphotoxin (LT) family members, in particular LTβ and LIGHT (also known as TNFSF14), and their downstream effectors, CCL17 and CCL20, were prominently overexpressed in human and mouse samples, along with signs of LT-driven non-canonical NF-κB pathway activation (FIG. 7A,B and FIG. 15A-D). LTβ is also expressed in ELSs of patients with chronic hepatitis (FIG. 7B,C). Moreover, a significant correlation was noted between CCL17 and CCL20 mRNA expression and that of LTβ in both human and mouse samples (FIG. 15E-H). This suggests that Lymphotoxin β receptor (LTβR) activation by LTα, LTβ and/or LIGHT could play a key role in ELS assembly and pro-tumorigenic processes (Tumanov A V, et al, Gastroenterology 2009, 136(2):694-704; Drutskaya M S, et al, Internation Union of Biochemistry and Molecular Biology Life 2010, 62(4):283-289; Bauer J, et al, Digestive Disease 2012, 30(5):453-468; Yun C, et al, Cancer Letters 2002, 184(1):97-104). LTβR was reported to be expressed on hepatocytes, whereas LTα and LTβ are normally expressed in lymphocytes (Yu G Y, et al. Molecular Cell 2012, 48(2):313-21). Indeed, mRNA in situ hybridization revealed that LTβ mRNA was expressed in immune, but not epithelial cells in small ELSs (FIG. 7D and FIG. 15I). To identify the specific cell types which express LTβ, flow sorting of single cell suspensions from ELSs was used. This analysis revealed the LTβ mRNA was expressed by both T and B lymphocytes, but not by hepatocytes (FIG. 15J). However, in advanced large ELSs, some of the neoplastic hepatocytes also expressed LTβ and all full-blown HCCs always express LTβ mRNA by histology (FIG. 7D and FIG. 15I). Notably, when varying degrees of expression were noted in malignant hepatocytes within an ELS, LTβ mRNA expression occurred in malignant hepatocytes at the ELS periphery, in particular within the egressing clusters (FIG. 7E,F and FIG. 15K,L). This raised the hypothesis that immune cells within the ELSs provide paracrine LT signals to early HCC progenitors, which are later replaced by an autocrine signal, allowing the malignant hepatocytes to gain independence from the niche. This presumption could be supported by the observation that transgenic overexpression of LTα and β in hepatocytes was found to induce HCC (Haybaeck J, et al. Cancer cell 2009, 16(4): 295-308). Importantly, HCCs that developed in IKKβ(EE)^{Hep} mice under anti-Thy1.2-treatment and in IKβ(EE)^{Hep}-Rag1^{-/-} livers showed marked reduction in LTβ mRNA expression, suggesting that exposure of tumor progenitors at early stages to niche-derived cytokines, renders them addicted to these cytokines, favoring acquisition of autocrine abilities to produce the same cytokines (FIG. 7G,H and FIG. 15M-P).

To test this hypothesis, LT cytokines were blocked using a soluble LTβR fused to a murine immunoglobulin Fc portion (LTβR-Ig) (Haybaeck, ibid.) . Furthermore, to denote the time point of when LT blockade had the most efficient biologic effect, IKKREEPP mice were subjected to three treatment regimens: 3-12 weeks of age, when LTR is expressed by ELS immune cells; 13-22 weeks of age, when LTβ is expressed by both immune and malignant ELS cells; and 23-32 weeks of age during which LTβ is expressed similarly to the intermediate period, yet HCCs are more developed (FIG. 16A,B). Measurements of expression of multiple cytokines in liver parenchyma showed reduction in many of the pro-inflammatory and of the LT-mediated cytokines (FIG. 8A). Of note, blocking LT signaling was associated with reduced NF-κB activation in ELS-residing malignant hepatocytes (FIG. 8C,D), suggesting that LT signaling enhanced the low level of NF-κB activation induced by the IKKβ(EE) transgene. Remarkably, LTβR-Ig treatment in early and intermediate periods dramatically reduced HCC burden (FIG. 8B and FIG. 16E,F). In contrast, LTβR-signaling inhibition at the late period resulted in a smaller, non-significant effect on HCC number and tumor volume. As LT inhibition proved ineffective in reducing tumorigenesis beyond 23 weeks of age, it was hypothesized that the major inhibitory effect of LT-blockade could have been in ELSs where LT is primarily provided by the lymphocytes rather than in an autocrine manner by niche-external tumor cells. Indeed, histological inspection of early tumorigenesis stages in the late treatment group revealed a marked reduction in the number of intra-ELS HCC progenitors, and in both multiplicity and size of clusters of egressing cells (FIG. 8C,D and FIG. 16G). This was associated with reduced proliferation of HCC progenitor cells in ELSs upon treatment (CDC47⁺Sox9⁺ doubly-labeled cells, FIG. 8E-G), possibly accounting for a lower number of ELS-egressing atypical hepatocytes (FIG. 8C,D).

To further test the therapeutic utility of ELS disruption, the CCL20/CCR6 axis was targeted. It was found that CCL20 is expressed by HCC progenitors in ELS. The only known receptor for CCL20 is CCR6. IKK(EE)^{hep} mice were bred with CCR6 knockout mice—this resulted in a marked reduction in ELS numbers, as well as in HCC incidence, in the offspring (vs. IKK(EE)^{hep} control mice). It thus appears that paracrine LT stimulation within ELSs is a critical step in HCC development, amenable to anti-tumor intervention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for predicting the likelihood of liver cancer or recurrence thereof in a subject in need thereof or for determining eligibility for anti-cancer therapy in a subject having liver cancer, the method comprising: determining at least one ectopic lymphoid-like structures (ELS)-related parameter in the subject, wherein a parameter higher than a predefined control indicates the subject has a high likelihood of developing liver cancer or that the subject is eligible for anti-cancer therapy.

2. (canceled)

3. The method of claim 1, wherein said subject has a pre-existing non-cancer pathological condition selected from the group consisting of: chronic liver inflammation, and fibrotic/cirrhotic liver conditions.

4. The method of claim 1, wherein determining at least one ELS-related parameter is determining the expression of at least one biomarker selected from the group consisting of: LTβ, CCL17 and CCL20.

5. The method of claim 1, wherein determining at least one ELS-related parameter is determining the expression of at least one biomarker selected from the group consisting of: LTβ, CCL17, CCL20, CCL21, CCL19, CXCL13, CXCL11, CCL8, CXCL10, CXCL9, CCL2, CCL3, CCL18, CCL4 and CCL5.

6. The method of claim 1, wherein determining at least one ELS-related parameter is determining the expression of at least one biomarker selected from the group consisting of: LTβ, CCL17 and CCL20, at one or more additional biomarkers selected from the group consisting of CCL21, CCL19, CXCL13, CXCL11, CCL8, CXCL10, CXCL9, CCL2, CCL3, CCL18, CCL4 and CCL5.

7. The method of claim 1, wherein said determining at least one ELS-related parameter is determining binding of an ELS-binding agent.

8. The method of claim 1, wherein said determining at least one ELS-related parameter is determining the presence or quantifying one or more ELS biomarkers.

9. The method of claim 1, wherein said determining comprises:
a. obtaining a biological sample from the subject; and
b. determining at least one ELS-related parameter in said biological sample.

10. The method of claim 9, wherein said biological sample is selected from the group consisting of: tissue, blood, serum, urine and cells.

11. The method of claim 1, wherein said biological sample is a biopsy derived from a tumor.

12. The method of claim 1, wherein said predefined control is selected from the group consisting of a non-cancerous sample from at least one individual, a panel of non-cancerous control samples from a set of individuals, and a stored set of data from control individuals.

13. The method of claim 1 for predicting the likelihood of recurrence of liver cancer in a subject after undergoing anti-cancer therapy, wherein a parameter higher than a predefined control is indicative that the subject has a high likelihood of late recurrence.

14. The method of claim 1 for determining eligibility for anti-cancer therapy, wherein a parameter higher than a predefined control is indicative that the subject is ineligible for treatment by inhibitory immune checkpoint drugs.

15. A method of determining the presence of ELS in the liver in a subject in need thereof, the method comprising:
a) obtaining a liver sample from the subject; and
b) determining the expression of at least one nucleic acid biomarker selected from the group consisting of: LTβ, CCL17 and CCL20, in said sample,
wherein expression higher than a predefined control indicates the presence of ELS in the liver of said subject.

16. A kit comprising one or more ligands or primers, each ligand or primer is capable of specifically complexing with, binding to, hybridizing to, or quantitatively detecting or identifying an ELS-related parameter.

17. The kit of claim 16, for use in determining ELS in a liver sample.

18. The kit of claim 16, wherein said one or more ligands are capable of specifically complexing with, binding to, hybridizing to, or quantitatively detecting or identifying one or more biomarkers selected from the group consisting of: LTβ, CCL20, CCL17, CCL21, CCL19, CXCL13, CXCL11, CCL8, CXCL10, CXCL9, CCL2, CCL3, CCL18, CCL5, CCL20 and CCL17.

19. A method for treating or reducing the likelihood of liver cancer in a subject in need thereof, the method comprising administering to said subject a therapeutically effective amount of anti-ELS agent, thereby treating or reducing the likelihood of liver cancer in said subj ect.

20. The method of claim 19, comprising the steps of:
a. determining liver-ELS in the subject;
b. administering to said subject a therapeutically effective amount of anti-ELS agent, thereby treating or reducing the likelihood of HCC in said subject.

21. The method of claim 1, wherein said anti-ELS agent is selected from the group consisting of anti-CD90 antibody, LTβR-IG, or CCR6 blockade.
